(19) United States
(12) Patent Application Publication
(10) Pub. No.: US 2024/0417754 A1
(43) Pub. Date: Dec. 19, 2024

(54) SERINE RECOMBINASES

(71) Applicants: The Regents of the University of California, Oakland, CA (US); The Board of Trustees of the Leland Stanford Junior University, Stanford, CA (US); Salk Institute for Biological Studies, La Jolla, CA (US)

(72) Inventors: Ami S. Bhatt, Stanford, CA (US); Matthew G. Durrant, Oakland, CA (US); Joshua C. Tycko, Stanford, CA (US); Patrick D. Hsu, Oakland, CA (US); Alison Fanton, Oakland, CA (US); Michael C. Bassik, Stanford, CA (US); Lacramioara Bintu, Stanford, CA (US)

(21) Appl. No.: 18/706,301

(22) PCT Filed: Nov. 3, 2022
(86) PCT No.: PCT/US22/79227

§ 371 Date: Apr. 30, 2024

Related U.S. Application Data
(60) Provisional application No. 63/400,868, filed on Aug. 25, 2022.
(60) Provisional application No. 63/322,712, filed on Mar. 23, 2022.
(60) Provisional application No. 63/275,288, filed on Nov. 3, 2021.

(51) Int. Cl. C12N 15/90; C12N 9/22

(52) U.S. Cl. CPC C12N 15/90; C12N 9/22; C12Y 301/22

(57) ABSTRACT

Provided herein are recombinases and compositions, methods of identification and methods of using thereof.

### CROSS-REFERENCE TO RELATED APPLICATIONS

This application claims the benefit of U.S. Provisional Application Nos. 63/275,288, filed Nov. 3, 2021, 63/322,712, filed Mar. 23, 2022, and 63/400,868, filed Aug. 25, 2022, the contents of which are herein incorporated by reference in their entirety.

### STATEMENT REGARDING FEDERALLY SPONSORED RESEARCH

This invention was made with government support under Grant Numbers OD021369 and AI148623 awarded by the National Institutes of Health. The government has certain rights in the invention.

### SEQUENCE LISTING STATEMENT

The contents of the electronic sequence listing titled 39817_601_SequenceListing.xml (Size: 3,888,144 bytes; and Date of Creation: Nov. 3, 2022) is herein incorporated by reference in its entirety.

### FIELD

The present invention relates to serine recombinases and methods of identification and use thereof.

### BACKGROUND

Despite recent advances in genome engineering, there remains a need for an efficient method to stably integrate multi-kilobase DNA cargos in human and other eukaryotic cells. Large serine recombinases (LSRs), such as BxB1 and ΦC31, have evolved to perform this task in microbial cells, but the previously characterized LSRs have several limitations not suited for use in genome engineering of eukaryotic cells. Directed evolution and protein engineering efforts have not yet successfully transformed these limited candidates into ideal molecular tools. New recombinases and methods of identifying the new recombinases are needed to expand the available tools for genetic engineering.

### SUMMARY

Provided herein are systems for DNA modification. In select embodiments, the system is a cell free system.

In some embodiments, the systems comprise a polypeptide comprising a recombinase having an amino acid sequence having at least 70% identity to any of SEQ ID NOs: 1-74, active fragments thereof, or a nucleic acid encoding thereof. In some embodiments, the recombinase has an amino acid sequence having at least 70% identity to any of SEQ ID NOs: 2, 6, 10, 12, 18, 19, 26, 29, 61, 65, or 66. In certain embodiments, the recombinase has an amino acid sequence of SEQ ID NOs: 2, 6, 10, 12, 18, 19, 26, 29, 61, 65, or 66.

In some embodiments, the systems a polypeptide comprising a recombinase having an amino acid sequence with at least 70% identity to one or more of the following:
1) X₁ₐX₂ₐX₃ₐX₄ₐX₅ₐX₆ₐX₇ₐX₈ₐX₉ₐX₁₀ₐX₁₁ₐX₁₂ₐX₁₃ₐX₁₄ₐX₁₅ₐX₁₆ₐX₁₇ₐX₁₈ₐX₁₉ₐX₂₀ₐX₂₁ₐX₂₂ₐX₂₃ₐX₂₄ₐX₂₅ₐX₂₆ₐX₂₇ₐX₂₈ₐX₂₉ₐX₃₀ₐX₃₁ₐX₃₂ₐX₃₃ₐX₃₄ₐ, wherein:
   X₁ₐ is A, E, I, L, S, T, V, or Y;
   X₂ₐ is A, D, E, G, K, Q, R, S, or T;
   X₆ₐ is E or G;
   X₈ₐ is A, C, F, L, M, or V;
   X₁₀ₐ is A, F, I, L, M, T, or V;
   X₁₃ₐ is F, H, I, L, M, N, or V;
   X₁₄ₐ is A, G, S, or V;
   X₁₅ₐ is A, D, I, L, S, T, or V;
   X₁₇ₐ is A, G, or S;
   X₂₁ₐ is K, R, S, or V;
   X₂₂ₐ is A, D, E, G, K, N, S, or T;
   X₂₃ₐ is A, E, I, K, M, N, Q, S, or T;
   X₂₄ₐ is F, I, L, M, S, or T;
   X₂₆ₐ is D, E, L, Q, S, or V;
   X₂₇ₐ is E, N, Q, or R;
   X₃₂ₐ is A, F, H, I, K, L, M, N, Q, R, S, or V
   X₃₄ₐ is A, E, G, H, K, L, M, N, Q, R, S, or V; and
   X₃ₐ, X₄ₐ, X₅ₐ, X₇ₐ, X₉ₐ, X₁₁ₐ, X₁₂ₐ, X₁₆ₐ, X₁₈ₐ, X₁₉ₐ, X₂₀ₐ, X₂₅ₐ, X₂₈ₐ, X₂₉ₐ, X₃₀ₐ, X₃₁ₐ, and X₃₃ₐ are each individually selected from any amino acid;
2) X_{1b}X_{2b}X_{3b}X_{4b}X_{5b}X_{6b}X_{7b}X_{8b}X_{9b}X_{10b}X_{11b}X_{12b}X_{13b}X_{14b}X_{15b}X_{16b}X_{17b}X_{18b}, wherein
   X_{1b} is A, G, or I;
   X_{2b} is D, E, G, N, P, S, T, or V;
   X_{3b} is D, G, N, Q, or S;
   X_{4b} is A, H, N, Q, R, T, V, or Y;
   X_{6b} is A, D, E, H, I, L, P, Q, R, T, or Y;
   X_{7b} is A, D, E, Q, or R;
   X_{8b} is F, I, K, or L;
   X_{10b} is D, E, F, G, N, Q, R, S, T, or V;
   X_{11b} is A, I, L, S, T, or V;
   X_{12b} is D, E, I, K, L, N, Q, R, S, T, or V;
   X_{13b} is A, D, E, K, M, N, R, S, T, or V;
   X_{14b} is A, G, Q, R, S, or T;
   X_{16b} is A, D, E, K, L, Q, R, or T; and
   X_{18b} is A, L, M, or V; and
   X_{5b}, X_{9b}, X_{15b}, and X_{17b} are each individually selected from any amino acid;
3) X_{1c}X_{2c}X_{3c}X_{4c}X_{5c}X_{6c}X_{7c}X_{8c}X_{9c}X_{10c}X_{11c}X_{12c}X_{13c}ESX_{16c}X_{17c}KX_{19c}X_{20c}X_{21c}X_{22c}X_{23c}X_{24c}X_{25c}X_{26c}, wherein
   X_{1c} is A, D, F, I, L, M, N, S, or Y;
   X_{4c} is A, I, K, M, S, or V;
   X_{6c} is A, F, G, I, L, M, or V;
   X_{10c} is Q, R, or T;
   X_{11c} is A, G, or S;
   X_{13c} is D, E, G, N, Q, or S;
   X_{17c} is A, H, K, N, R, S, T, or V;
   X_{21c} is L, M, R, or Y;
   X_{22c} is A, I, N, Q, S, T, or V;
   X_{23c} is A, E, F, I, K, L, N, R, T, or V;
   X_{25c} is A, F, H, L, N, Q, S, T, or Y;
   X_{26c} is A, I, L, M, N, R, S, T, V, or Y; and
   X_{2c}, X_{3c}, X_{5c}, X_{7c}, X_{8c}, X_{9c}, X_{12c}, X_{16c}, X_{19c}, X_{20c}, and X_{24c} are each individually selected from any amino acid;
4) X_{1d}X_{2d}X_{3d}X_{4d}X_{5d}X_{6d}X_{7d}X_{8d}X_{9d}X_{10d}X_{11d}X_{12d}X_{13d}X_{14d}X_{15d}X_{16d}X_{17d}X_{18d}X_{19d}X_{20d}X_{21d}X_{22d}X_{23d}X_{24d}X_{25d}X_{26d}X_{27d}X_{28d}, wherein:
   X_{1d} is E, K, N, T, G, S, L, D, V, A, R, or P;
   X_{2d} is E, H, I, T, G, S, L, D, V, A, or P;
   X_{4d} is M, I, T, S, L, V, A, R or P;
   X_{5d} is E, K, N, I, T, G, S, D, Q, V, A, R, or P;
   X_{6d} is E, G, S, D, A, R, or P;
   X_{7d} is I, L, D, A, or R;
   X_{8d} is M, H, K, T, L, V, Q, D, A, or R;
   X_{9d} is E, K, I, T, G, S, L, D, Q, V, or A;
   X_{10d} is E, K, H, D, Q, V, A, or R;
   X_{11d} is M, H, I, S, L, V, Q, A, or R;
   X_{12d} is Q, E, K, N, M, S, L, D, V, A, or R;
   X_{13d} is E, K, H, G, S, L, D, Q, A, or R;
   X_{14d} is E, Y, K, N, I, H, L, V, or A;
   X_{16d} is E, K, I, T, G, S, L, D, Q, A, or R;
   X_{17d} is E, K, H, T, G, D, Q, A, or R;
   X_{19d} is Q, E, K, N, T, G, S, D, V, A, or R;
   X_{20d} is Q, E, K, N, T, G, S, V, D, A, or R;
   X_{21d} is I, S, W, L, V, F, A, or R;
   X_{22d} is Q, E, M, T, G, S, L, V, D, or A;
   X_{23d} is E, K, N, I, T, G, S, D, A, R, or P;
   X_{24d} is E, M, I, L, D, Q, or A;
   X_{25d} is E, Y, I, L, V, F, A, or R;
   X_{26d} is E, M, T, G, S, L, D, V, A, or R;
   X_{27d} is E, K, N, G, S, L, D, Q, A, or R;
   X_{28d} is Q, E, G, V, D, A, R, or P; and
   X_{3d}, X_{15d}, and X_{18d} are each individually selected from any amino acid;
5) X₁ₑX₂ₑX₃ₑX₄ₑX₅ₑX₆ₑX₇ₑX₈ₑX₉ₑX₁₀ₑX₁₁ₑX₁₂ₑX₁₃ₑX₁₄ₑX₁₅ₑX₁₆ₑX₁₇ₑX₁₈ₑ, wherein:
   X₁ₑ is A, D, E, H, K, N, Q, R, or S;
   X₂ₑ is A, D, E, F, G, H, K, M, N, Q, R, S, W, or Y;
   X₃ₑ is E, F, or Y;
   X₄ₑ is F, H, L, W, or Y;
   X₆ₑ is A, D, E, F, I, K, L, M, N, Q, R, S, T, or Y;
   X₇ₑ is F, I, Q, S, T, or V;
   X₈ₑ is A, G, K, L, N, R, S, T, or V;
   X₉ₑ is A, D, E, H, K, N, Q, R, T, or Y;
   X₁₀ₑ is I, N, Q, or R;
   X₁₁ₑ is F, I, L, M, Q, or S;
   X₁₄ₑ is A, G, K, N, or S;
   X₁₅ₑ is K, M, Q, R, S, T, or V;
   X₁₈ₑ is A, E, G, K, M, N, S, T, or Y; and
   X₅ₑ, X₁₂ₑ, X₁₃ₑ, X₁₆ₑ, and X₁₇ₑ are each individually selected from any amino acid;
6) WX_{2f}X_{3f}X_{4f}X_{5f}X_{6f}X_{7f}X_{8f}X_{9f}X_{10f}X_{11f}X_{12f}X_{13f}X_{14f}X_{15f}X_{16f}GX_{18f}X_{19f}X_{20f}X_{21f}X_{22f}X_{23f}, wherein:
   X_{2f} is A, E, H, N, R, S, T, or V;
   X_{4f} is A, G, N, S, or T;
   X_{5f} is F, G, L, M, N, Q, S, T, or V;
   X_{6f} is I, L, P, or V;
   X_{9f} is I, L, T, or V;
   X_{14f} is A, C, G, M, Q, R, S, or T;
   X_{16f} is I, L, V, or Y;
   X_{18f} is D, E, H, N, Q, or S;
   X_{20f} is E, H, I, L, M, Q, R. or T;
   X_{21f} is A, E, F, H, L, N, P, or Y;
   X_{22f} is C, F, H, K, M, N, Q, R, T, or Y;
   X_{23f} is D, E, F, I, K, L, N, Q, R, S, T, or V; and
   X_{3f}, X_{7f}, X_{8f} X_{10f}, X_{11f}, X_{12f}, X_{13f}, X_{15f}, and X_{19f} are each individually selected from any amino acid;
7) X_{1g}X_{2g}X_{3g}X_{4g}X_{5g}EX_{7g}X_{8g}X_{9g}X_{10g}X_{11g}X_{12g}RX_{14g}X_{15g}X_{16g}X_{17g}X_{18g}X_{19g}X_{20g}X_{21g}, wherein:
   X_{1g} is A, G, I, N, S, T, or V;
   X_{3g} is A, I, or S;
   X_{5g} is F, I, L, M, or Y;
   X_{7g} is I or R;
   X_{10g} is D, I, L, or T;
   X_{12g} is A, E, I, K, M, Q, or S;
   X_{14g} is I, T, or V;
   X_{16g} is A, D, G, R, S, or T;
   X_{18g} is F, K, L, M, or Y;
   X_{19g} is A, E, H, I, K, L, M, N, Q, R, V, W, or Y;
   X_{21g} is A, I, K, L, M, or R; and
   X_{2g}, X_{4g}, X_{8g}, X_{9g}, X_{11g}, X_{15g}, X_{17g}, and X_{20g} are each individually selected from any amino acid;
8) X₁ₕX₂ₕX₃ₕX₄ₕX₅ₕX₆ₕX₇ₕX₈ₕX₉ₕX₁₀ₕX₁₁ₕ, wherein:
   X₁ₕ is F or Y;
   X₂ₕ is D, E, K, Q, or S;
   X₃ₕ is E, K, L, M, or Q;
   X₄ₕ is K, L, or R;
   X₅ₕ is K, L, or V;
   X₇ₕ is G or N;
   X₈ₕ is D, E, H, K, L, M, or R;
   X₉ₕ is S or T;
   X₁₁ₕ is F, H, I, Q, S, T, V, or W; and
   X₆ₕ and X₁₀ₕ are each individually selected from any amino acid;
9) X₁ᵢX₂ᵢX₃ᵢX₄ᵢX₅ᵢX₆ᵢX₇ᵢX₈ᵢX₉ᵢX₁₀ᵢX₁₁ᵢX₁₃ᵢX₁₄ᵢX₁₅ᵢX₁₆ᵢX₁₇ᵢX₁₈ᵢX₁₉ᵢX₂₀ᵢX₂₁ᵢX₂₂ᵢX₂₃ᵢX₂₄ᵢX₂₅ᵢX₂₆ᵢX₂₇ᵢ, wherein:
   X₁ᵢ is I, L, or V;
   X₄ᵢ is A, D, F, H, I, L, M, N, Q, S, V, or Y;
   X₈ᵢ is A, G, or S;
   X₁₀ᵢ is D, E, I, K, N, Q, R, or S;
   X₁₁ᵢ is E or Q;
   X₁₅ᵢ is A or K;
   X₁₆ᵢ is A, Q, R, or S;
   X₁₈ᵢ is L, M, or R;
   X₁₉ᵢ is I, L, Q, R, S, or V;
   X₂₁ᵢ is A, D, E, G, H, I, Q. R, or S;
   X₂₂ᵢ is A, K, N, Q, S, T, or V;
   X₂₃ᵢ is A, H, K, R, W, or Y;
   X₂₅ᵢ is A, G, H, I, K, Q, R, S, or T;
   X₂₇ᵢ is C, H, I, K, L, R, or V; and
   X₂ᵢ, X₃ᵢ, X₅ᵢ, X₆ᵢ, X₇ᵢ, X₉ᵢ, X₁₃ᵢ, X₁₄ᵢ, X₁₇ᵢ, X₂₀ᵢ, X₂₄ᵢ, and X₂₆ᵢ are each individually selected from any amino acid;
10) RX₂ⱼX₃ⱼX₄ⱼW, wherein:
   X₂ⱼ is L, M, Q, or R;
   X₃ⱼ is A, N, or S; and
   X₄ⱼ is N, P, S, or T;
11) X₁ₖX₂ₖX₃ₖX₄ₖX₅ₖX₆ₖX₇ₖX₈ₖF, wherein:
   X₁ₖ is I, L, or V;
   X₂ₖ is A or V;
   X₄ₖ is A, F, H, I, L, Q, W, or Y;
   X₅ₖ is I, M, or V;
   X₇ₖ is E, L, Q, or T;
   X₈ₖ is A, I, or V; and
   X₃ₖ and X₆ₖ are each individually selected from any amino acid;
12) RX₂ₗX₃ₗX₄ₗX₅ₗX₆ₗX₇ₗX₈ₗX₉ₗX₁₀ₗX₁₁ₗX₁₂ₗX₁₃ₗ, wherein:
   X₂ₗ is D, K, N, R, S, or V;
   X₃ₗ is A, D, E, F, G, K, P, Q, or S;
   X₄ₗ is A, E, I, K, L, S, T, or V;
   X₅ₗ is any amino acid;
   X₆ₗ is F, G, I, L, N, or V;
   X₇ₗ is A, F, I, L, Q, R, V, or Y;
   X₈ₗ is D, E, I, L, M, N, Q, S, T, or V;
   X₉ₗ is D, E, F, I, L, M, Q, T, V, or Y;
   X₁₀ₗ is I, K, L, R, or V;
   X₁₁ₗ is D, E, K, N, Q, or R;
   X₁₂ₗ is D, E, F, K, L, N, Q, W, or Y; and
   X₁₃ₗ is F or L; and
13) X₁ₘX₂ₘX₃ₘX₄ₘX₅ₘX₆ₘX₇ₘX₈ₘX₉ₘX₁₀ₘX₁₁ₘX₁₂ₘX₁₃ₘX₁₄ₘX₁₅ₘX₁₆ₘX₁₇ₘX₁₈ₘX₁₉ₘX₂₀ₘX₂₁ₘX₂₂ₘX₂₃ₘX₂₄ₘ, wherein:
   X₁ₘ is A, E, F, I, L, M, N, Q, S, T, V, or Y;
   X₂ₘ is A, F, G, I, L, M, R, S, T, or V;
   X₆ₘ is A, D, E, F, G, H, L, M, N, S, or T;
   X₉ₘ is D, M, N, or S;
   X₁₀ₘ is D, E, or Q;
   X₁₂ₘ is C, F, H, L, T, V, or Y;
   X₁₄ₘ is A, E, K, L, R, or Y;
   X₁₇ₘ is A, L, or S;
   X₁₉ₘ is D, E, K, N, Q, R, or S;
   X₂₀ₘ is G, I, M, Q, R, T, or V;
   X₂₁ₘ is D, H, K, N, Q, or R;
   X₂₃ₘ is A, G, I, L, N, S, T, or V;
   X₂₄ₘ is F, H, I, K, L, M, N, Q, V, W, or Y; and
   X₃ₘ, X₄ₘ, X₅ₘ, X₇ₘ, X₈ₘ, X₁₁ₘ, X₁₃ₘ, X₁₅ₘ, X₁₆ₘ, X₁₈ₘ, and X₂₂ₘ, are each individually selected from any amino acid,

or active fragments thereof, or a nucleic acid encoding thereof; and

a first polynucleotide comprising a donor recognition sequence for the recombinase.

In some embodiments, the systems comprise a polypeptide comprising a recombinase having an amino acid sequence having at least 70% identity to SEQ ID NOs: 88-1183.

The systems may further comprise a first polynucleotide comprising a donor recognition sequence for the recombinase. In some embodiments, the donor recognition sequence comprises a donor attachment site configured to bind the recombinase. Recognition sites are polynucleotide sequences that comprise any and all sequence elements facilitating recognition by the recombinase enzyme. Attachment sites are those specific polynucleotide sequences that where recombination occurs.

In some embodiments, the first polynucleotide further comprises a cargo DNA sequence, which is a polynucleotide that is to be delivered or inserted into a target sequence. The cargo DNA sequence may be greater than 1 kilobase pair (e.g., greater than 2 kilobase pairs, greater than 4 kilobase pairs, greater than 6 kilobase pairs, greater than 8 kilobase pairs, greater than 10 kilobase pairs, greater than 15 kilobase pairs, greater than 20 kilobase pairs, or more). In select embodiments, the cargo DNA sequence is greater than 5 kilobase pairs.

In some embodiments, the first polynucleotide further comprises a recipient recognition sequence for the recombinase. In some embodiments, the system further comprises a second polynucleotide comprising a recipient recognition sequence for the recombinase. In some embodiments, the recipient recognition sequence comprises a recipient attachment sequence configured to bind to the recombinase.

In some embodiments, the donor recognition sequence, the recipient recognition sequence, or both are pseudo-recognition sequences. Pseudo-recognition sequences” or “pseudosites” refer to a recognition sequences which is not necessarily that which is the native recognition sequence for a given recombinase but rather is sufficient to promote recombination.

Also provided herein are compositions and cells comprising the disclosed system. In some embodiments, the cell is a eukaryotic cell.

Further provided herein are methods for altering a target DNA.

In some embodiments, the methods comprise contacting the target DNA with a polypeptide comprising a recombinase having an amino acid sequence having at least 70% identity to any of SEQ ID NOs: 1-74, active fragments thereof, or a nucleic acid encoding thereof. In some embodiments, the recombinase has an amino acid sequence having at least 70% identity to any of SEQ ID NOs: 2, 6, 10, 12, 18, 19, 26, 29, 61, 65, or 66. In certain embodiments, the recombinase has an amino acid sequence of SEQ ID NOs: 2, 6, 10, 12, 18, 19, 26, 29, 61, 65, or 66.

In some embodiments, the methods comprise contacting the target DNA with a polypeptide comprising a recombinase having an amino acid sequence with at least 70% identity to one or more of the following:
1) X₁ₐX₂ₐX₃ₐX₄ₐX₅ₐX₆ₐX₇ₐX₈ₐX₉ₐX₁₀ₐX₁₁ₐX₁₂ₐX₁₃ₐX₁₄ₐX₁₅ₐX₁₆ₐX₁₇ₐX₁₈ₐX₁₉ₐX₂₀ₐX₂₁ₐX₂₂ₐX₂₃ₐX₂₄ₐX₂₅ₐX₂₆ₐX₂₇ₐX₂₈ₐX₂₉ₐX₃₀ₐX₃₁ₐX₃₂ₐX₃₃ₐX₃₄ₐ, wherein:
   X₁ₐ is A, E, I, L, S, T, V, or Y;
   X₂ₐ is A, D, E, G, K, Q, R, S, or T;
   X₆ₐ is E or G;
   X₈ₐ is A, C, F, L, M, or V;
   X₁₀ₐ is A, F, I, L, M, T, or V;
   X₁₃ₐ is F, H, I, L, M, N, or V;
   X₁₄ₐ is A, G, S, or V;
   X₁₅ₐ is A, D, I, L, S, T, or V;
   X₁₇ₐ is A, G, or S;
   X₂₁ₐ is K, R, S, or V;
   X₂₂ₐ is A, D, E, G, K, N, S, or T;
   X₂₃ₐ is A, E, I, K, M, N, Q, S, or T;
   X₂₄ₐ is F, I, L, M, S, or T;
   X₂₆ₐ is D, E, L, Q, S, or V;
   X₂₇ₐ is E, N, Q, or R;
   X₃₂ₐ is A, F, H, I, K, L, M, N, Q, R, S, or V
   X₃₄ₐ is A, E, G, H, K, L, M, N, Q, R, S, or V; and
   X₃ₐ, X₄ₐ, X₅ₐ, X₇ₐ, X₉ₐ, X₁₁ₐ, X₁₂ₐ, X₁₆ₐ, X₁₈ₐ, X₁₉ₐ, X₂₀ₐ, X₂₅ₐ, X₂₈ₐ, X₂₉ₐ, X₃₀ₐ, X₃₁ₐ, and X₃₃ₐ are each individually selected from any amino acid;
2) X_{1b}X_{2b}X_{3b}X_{4b}X_{5b}X_{6b}X_{7b}X_{8b}X_{9b}X_{10b}X_{11b}X_{12b}X_{13b}X_{14b}X_{15b}X_{16b}X_{17b}X_{18b}, wherein
   X_{1b} is A, G, or I;
   X_{2b} is D, E, G, N, P, S, T, or V;
   X_{3b} is D, G, N, Q, or S;
   X_{4b} is A, H, N, Q, R, T, V, or Y;
   X_{6b} is A, D, E, H, I, L, P, Q, R, T, or Y;
   X_{7b} is A, D, E, Q, or R;
   X_{8b} is F, I, K, or L;
   X_{10b} is D, E, F, G, N, Q, R, S, T, or V;
   X_{11b} is A, I, L, S, T, or V;
   X_{12b} is D, E, I, K, L, N, Q, R, S, T, or V;
   X_{13b} is A, D, E, K, M, N, R, S, T, or V;
   X_{14b} is A, G, Q, R, S, or T;
   X_{16b} is A, D, E, K, L, Q, R, or T; and
   X_{18b} is A, L, M, or V; and
   X_{5b}, X_{9b}, X_{15b}, and X_{17b} are each individually selected from any amino acid;
3) X_{1c}X_{2c}X_{3c}X_{4c}X_{5c}X_{6c}X_{7c}X_{8c}X_{9c}X_{10c}X_{11c}X_{12c}X_{13c}ESX_{16c}X_{17c}KX_{19c}X_{20c}X_{21c}X_{22c}X_{23c}X_{24c}X_{25c}X_{26c}, wherein
   X_{1c} is A, D, F, I, L, M, N, S, or Y;
   X_{4c} is A, I, K, M, S, or V;
   X_{6c} is A, F, G, I, L, M, or V;
   X_{10c} is Q, R, or T;
   X_{11c} is A, G, or S;
   X_{13c} is D, E, G, N, Q, or S;
   X_{17c} is A, H, K, N, R, S, T, or V;
   X_{21c} is L, M, R, or Y;
   X_{22c} is A, I, N, Q, S, T, or V;
   X_{23c} is A, E, F, I, K, L, N, R, T, or V;
   X_{25c} is A, F, H, L, N, Q, S, T, or Y;
   X_{26c} is A, I, L, M, N, R, S, T, V, or Y; and
   X_{2c}, X_{3c}, X_{5c}, X_{7c}, X_{8c}, X_{9c}, X_{12c}, X_{16c}, X_{19c}, X_{20c}, and X_{24c} are each individually selected from any amino acid;
4) X_{1d}X_{2d}X_{3d}X_{4d}X_{5d}X_{6d}X_{7d}X_{8d}X_{9d}X_{10d}X_{11d}X_{12d}X_{13d}X_{14d}X_{15d}X_{16d}X_{17d}X_{18d}X_{19d}X_{20d}X_{21d}X_{22d}X_{23d}X_{24d}X_{25d}X_{26d}X_{27d}X_{28d}, wherein:
   X_{1d} is E, K, N, T, G, S, L, D, V, A, R, or P;
   X_{2d} is E, H, I, T, G, S, L, D, V, A, or P;
   X_{4d} is M, I, T, S, L, V, A, R or P;
   X_{5d} is E, K, N, I, T, G, S, D, Q, V, A, R, or P;
   X_{6d} is E, G, S, D, A, R, or P;
   X_{7d} is I, L, D, A, or R;
   X_{8d} is M, H, K, T, L, V, Q, D, A, or R;
   X_{9d} is E, K, I, T, G, S, L, D, Q, V, or A;
   X_{10d} is E, K, H, D, Q, V, A, or R;
   X_{11d} is M, H, I, S, L, V, Q, A, or R;
   X_{12d} is Q, E, K, N, M, S, L, D, V, A, or R;
   X_{13d} is E, K, H, G, S, L, D, Q, A, or R;
   X_{14d} is E, Y, K, N, I, H, L, V, or A;
   X_{16d} is E, K, I, T, G, S, L, D, Q, A, or R;
   X_{17d} is E, K, H, T, G, D, Q, A, or R;
   X_{19d} is Q, E, K, N, T, G, S, D, V, A, or R;
   X_{20d} is Q, E, K, N, T, G, S, V, D, A, or R;
   X_{21d} is I, S, W, L, V, F, A, or R;
   X_{22d} is Q, E, M, T, G, S, L, V, D, or A;
   X_{23d} is E, K, N, I, T, G, S, D, A, R, or P;
   X_{24d} is E, M, I, L, D, Q, or A;
   X_{25d} is E, Y, I, L, V, F, A, or R;
   X_{26d} is E, M, T, G, S, L, D, V, A, or R;
   X_{27d} is E, K, N, G, S, L, D, Q, A, or R;
   X_{28d} is Q, E, G, V, D, A, R, or P; and
   X_{3d}, X_{15d}, and X_{18d} are each individually selected from any amino acid;
5) X₁ₑX₂ₑX₃ₑX₄ₑX₅ₑX₆ₑX₇ₑX₈ₑX₉ₑX₁₀ₑX₁₁ₑX₁₂ₑX₁₃ₑX₁₄ₑX₁₅ₑX₁₆ₑX₁₇ₑX₁₈ₑ, wherein:
   X₁ₑ is A, D, E, H, K, N, Q, R, or S;
   X₂ₑ is A, D, E, F, G, H, K, M, N, Q, R, S, W, or Y;
   X₃ₑ is E, F, or Y;
   X₄ₑ is F, H, L, W, or Y;
   X₆ₑ is A, D, E, F, I, K, L, M, N, Q, R, S, T, or Y;
   X₇ₑ is F, I, Q, S, T, or V;
   X₈ₑ is A, G, K, L, N, R, S, T, or V;
   X₉ₑ is A, D, E, H, K, N, Q, R, T, or Y;
   X₁₀ₑ is I, N, Q, or R;
   X₁₁ₑ is F, I, L, M, Q, or S;
   X₁₄ₑ is A, G, K, N, or S;
   X₁₅ₑ is K, M, Q, R, S, T, or V;
   X₁₈ₑ is A, E, G, K, M, N, S, T, or Y; and
   X₅ₑ, X₁₂ₑ, X₁₃ₑ, X₁₆ₑ, and X₁₇ₑ are each individually selected from any amino acid;
6) WX_{2f}X_{3f}X_{4f}X_{5f}X_{6f}X_{7f}X_{8f}X_{9f}X_{10f}X_{11f}X_{12f}X_{13f}X_{14f}X_{15f}X_{16f}GX_{18f}X_{19f}X_{20f}X_{21f}X_{22f}X_{23f}, wherein:
   X_{2f} is A, E, H, N, R, S, T, or V;
   X_{4f} is A, G, N, S, or T;
   X_{5f} is F, G, L, M, N, Q, S, T, or V;
   X_{6f} is I, L, P, or V;
   X_{9f} is I, L, T, or V;
   X_{14f} is A, C, G, M, Q, R, S, or T;
   X_{16f} is I, L, V, or Y;
   X_{18f} is D, E, H, N, Q, or S;
   X_{20f} is E, H, I, L, M, Q, R. or T;
   X_{21f} is A, E, F, H, L, N, P, or Y;
   X_{22f} is C, F, H, K, M, N, Q, R, T, or Y;
   X_{23f} is D, E, F, I, K, L, N, Q, R, S, T, or V; and
   X_{3f}, X_{7f}, X_{8f} X_{10f}, X_{11f}, X_{12f}, X_{13f}, X_{15f}, and X_{19f} are each individually selected from any amino acid;
7) X_{1g}X_{2g}X_{3g}X_{4g}X_{5g}EX_{7g}X_{8g}X_{9g}X_{10g}X_{11g}X_{12g}RX_{14g}X_{15g}X_{16g}X_{17g}X_{18g}X_{19g}X_{20g}X_{21g}, wherein:
   X_{1g} is A, G, I, N, S, T, or V;
   X_{3g} is A, I, or S;
   X_{5g} is F, I, L, M, or Y;
   X_{7g} is I or R;
   X_{10g} is D, I, L, or T;
   X_{12g} is A, E, I, K, M, Q, or S;
   X_{14g} is I, T, or V;
   X_{16g} is A, D, G, R, S, or T;
   X_{18g} is F, K, L, M, or Y;
   X_{19g} is A, E, H, I, K, L, M, N, Q, R, V, W, or Y;
   X_{21g} is A, I, K, L, M, or R; and
   X_{2g}, X_{4g}, X_{8g}, X_{9g}, X_{11g}, X_{15g}, X_{17g}, and X_{20g} are each individually selected from any amino acid;
8) X₁ₕX₂ₕX₃ₕX₄ₕX₅ₕX₆ₕX₇ₕX₈ₕX₉ₕX₁₀ₕX₁₁ₕ, wherein:
   X₁ₕ is F or Y;
   X₂ₕ is D, E, K, Q, or S;
   X₃ₕ is E, K, L, M, or Q;
   X₄ₕ is K, L, or R;
   X₅ₕ is K, L, or V;
   X₇ₕ is G or N;
   X₈ₕ is D, E, H, K, L, M, or R;
   X₉ₕ is S or T;
   X₁₁ₕ is F, H, I, Q, S, T, V, or W; and
   X₆ₕ and X₁₀ₕ are each individually selected from any amino acid;
9) X₁ᵢX₂ᵢX₃ᵢX₄ᵢX₅ᵢX₆ᵢX₇ᵢX₈ᵢX₉ᵢX₁₀ᵢX₁₁ᵢX₁₃ᵢX₁₄ᵢX₁₅ᵢX₁₆ᵢX₁₇ᵢX₁₈ᵢX₁₉ᵢX₂₀ᵢX₂₁ᵢX₂₂ᵢX₂₃ᵢX₂₄ᵢX₂₅ᵢX₂₆ᵢX₂₇ᵢ, wherein:
   X₁ᵢ is I, L, or V;
   X₄ᵢ is A, D, F, H, I, L, M, N, Q, S, V, or Y;
   X₈ᵢ is A, G, or S;
   X₁₀ᵢ is D, E, I, K, N, Q, R, or S;
   X₁₁ᵢ is E or Q;
   X₁₅ᵢ is A or K;
   X₁₆ᵢ is A, Q, R, or S;
   X₁₈ᵢ is L, M, or R;
   X₁₉ᵢ is I, L, Q, R, S, or V;
   X₂₁ᵢ is A, D, E, G, H, I, Q. R, or S;
   X₂₂ᵢ is A, K, N, Q, S, T, or V;
   X₂₃ᵢ is A, H, K, R, W, or Y;
   X₂₅ᵢ is A, G, H, I, K, Q, R, S, or T;
   X₂₇ᵢ is C, H, I, K, L, R, or V; and
   X₂ᵢ, X₃ᵢ, X₅ᵢ, X₆ᵢ, X₇ᵢ, X₉ᵢ, X₁₃ᵢ, X₁₄ᵢ, X₁₇ᵢ, X₂₀ᵢ, X₂₄ᵢ, and X₂₆ᵢ are each individually selected from any amino acid;
10) RX₂ⱼX₃ⱼX₄ⱼW, wherein:
   X₂ⱼ is L, M, Q, or R;
   X₃ⱼ is A, N, or S; and
   X₄ⱼ is N, P, S, or T;
11) X₁ₖX₂ₖX₃ₖX₄ₖX₅ₖX₆ₖX₇ₖX₈ₖF, wherein:
   X₁ₖ is I, L, or V;
   X₂ₖ is A or V;
   X₄ₖ is A, F, H, I, L, Q, W, or Y;
   X₅ₖ is I, M, or V;
   X₇ₖ is E, L, Q, or T;
   X₈ₖ is A, I, or V; and
   X₃ₖ and X₆ₖ are each individually selected from any amino acid;
12) RX₂ₗX₃ₗX₄ₗX₅ₗX₆ₗX₇ₗX₈ₗX₉ₗX₁₀ₗX₁₁ₗX₁₂ₗX₁₃ₗ, wherein:
   X₂ₗ is D, K, N, R, S, or V;
   X₃ₗ is A, D, E, F, G, K, P, Q, or S;
   X₄ₗ is A, E, I, K, L, S, T, or V;
   X₅ₗ is any amino acid;
   X₆ₗ is F, G, I, L, N, or V;
   X₇ₗ is A, F, I, L, Q, R, V, or Y;
   X₈ₗ is D, E, I, L, M, N, Q, S, T, or V;
   X₉ₗ is D, E, F, I, L, M, Q, T, V, or Y;
   X₁₀ₗ is I, K, L, R, or V;
   X₁₁ₗ is D, E, K, N, Q, or R;
   X₁₂ₗ is D, E, F, K, L, N, Q, W, or Y; and
   X₁₃ₗ is F or L; and
13) X₁ₘX₂ₘX₃ₘX₄ₘX₅ₘX₆ₘX₇ₘX₈ₘX₉ₘX₁₀ₘX₁₁ₘX₁₂ₘX₁₃ₘX₁₄ₘX₁₅ₘX₁₆ₘX₁₇ₘX₁₈ₘX₁₉ₘX₂₀ₘX₂₁ₘX₂₂ₘX₂₃ₘX₂₄ₘ, wherein:
   X₁ₘ is A, E, F, I, L, M, N, Q, S, T, V, or Y;
   X₂ₘ is A, F, G, I, L, M, R, S, T, or V;
   X₆ₘ is A, D, E, F, G, H, L, M, N, S, or T;
   X₉ₘ is D, M, N, or S;
   X₁₀ₘ is D, E, or Q;
   X₁₂ₘ is C, F, H, L, T, V, or Y;
   X₁₄ₘ is A, E, K, L, R, or Y;
   X₁₇ₘ is A, L, or S;
   X₁₉ₘ is D, E, K, N, Q, R, or S;
   X₂₀ₘ is G, I, M, Q, R, T, or V;
   X₂₁ₘ is D, H, K, N, Q, or R;
   X₂₃ₘ is A, G, I, L, N, S, T, or V;
   X₂₄ₘ is F, H, I, K, L, M, N, Q, V, W, or Y; and
   X₃ₘ, X₄ₘ, X₅ₘ, X₇ₘ, X₈ₘ, X₁₁ₘ, X₁₃ₘ, X₁₅ₘ, X₁₆ₘ, X₁₈ₘ, and X₂₂ₘ, are each individually selected from any amino acid,

or active fragments thereof, or a nucleic acid encoding thereof.

In some embodiments, the methods comprise contacting the target DNA with a polypeptide comprising a recombinase having an amino acid sequence having at least 70% identity to any of SEQ ID NOs: 88-1183, active fragments thereof, or a nucleic acid encoding thereof.

In some embodiments, the target DNA comprises a donor recognition sequence, a recipient recognition sequence, or both. In certain embodiments, the target DNA comprises a recipient attachment sequence configured to bind to the recombinase.

In some embodiments, the method further comprises contacting the target DNA with a first polynucleotide comprising a donor recognition sequence for the recombinase.

In some embodiments, the first polynucleotide further comprises a cargo DNA sequence. The cargo DNA sequence may be greater than 1 kilobase pair (e.g., greater than 2 kilobase pairs, greater than 4 kilobase pairs, greater than 6 kilobase pairs, greater than 8 kilobase pairs, greater than 10 kilobase pairs, greater than 15 kilobase pairs, greater than 20 kilobase pairs, or more). In select embodiments, the cargo DNA sequence is greater than 5 kilobase pairs.

In some embodiments, the donor recognition sequence, the recipient recognition sequence, or both are pseudo-recognition sequences.

In some embodiments, the target DNA sequence encodes a gene product. In certain embodiments, the target DNA sequence is a genomic DNA sequence.

In some embodiments, the target DNA is in a cell. In certain embodiments, the cell is a eukaryotic cell (e.g., a human or plant cell). In certain embodiments, the cell is a prokaryotic cell.

In some embodiments, the contacting comprises introducing one or more components of the system into the cell. In some embodiments, the recombinase, or the nucleic acid encoding thereof, is introduced into the cell before, concurrently with, or after the introduction of the donor polynucleotide.

In some embodiments, introducing into the cell comprises administering one or more components of the system to a subject (e.g., a human). In certain embodiments, the administering comprises in vivo administration. In certain embodiments, the administering comprises transplantation of ex vivo treated cells comprising one or more components of the system.

Other aspects and embodiments of the disclosure will be apparent in light of the following detailed description.

### BRIEF DESCRIPTION OF THE DRAWINGS

FIGS. 1A-1H show the systematic identification of thousands of recombinases and their predicted attachment sites for site-specific and multi-targeting/transposable clades. FIG. 1A is a schematic of a of computational workflow to identify LSRs and attachment sites. Briefly, protein sequences contained in RefSeq and GenBank bacterial isolate genomes were searched to identify sequences containing a “Recombinase” (PF07508) domain. Genomes that contained such a protein were compared with genomes that lacked this protein to determine if the recombinase resided on an integrated mobile genetic element. Once the boundaries of this MGE were identified, the original attachment sites were reconstituted by inspecting the sequences flanking these boundaries. This workflow was an extension of previous smaller scale computational methods (Yang et al. 2014 Nat Methods. 11(12): 1261-1266, incorporated herein by reference in its entirety). FIG. 1B is a phylogenetic tree of the amino acid sequences of representatives of LSR families annotated according to predicted target specificity of each LSR cluster. The figure legend “Unique Integration Targets” specifies the number of predicted target protein families that each LSR cluster is found to target in the database. Families labeled with “1” were identified using the technique described in FIG. 1C. Families labeled “2”, “3”, or “>3” were identified as described in panel FIG. 1F. A prominent multi-targeting clade is apparent in the top right portion of the phylogenetic tree shown here. The size of each point indicates the number of unique sequences found in each LSR cluster. FIG. 1C is a schematic of an exemplary technique to identify site-specific LSRs. Briefly, when multiple LSR clusters (clustered at 50% identity) integrate into a single gene cluster (clustered at 50% identity), then all LSR families are considered site-specific. The typical domain architecture of a site-specific LSR is shown on the right, including the Resolvase (green). Recombinase (red), and the Recombinase zinc beta ribbon domain (purple). FIG. 1D is an exemplary observed network of predicted site-specific LSRs. Each node indicates either an LSR cluster (red) or a target protein cluster (blue). Edges between nodes indicate that at least one member of the target protein cluster was found to integrate into at least one member of the target protein cluster. FIG. 1E is an exemplary hierarchical tree of diverse LSR sequences that target a set of closely related attB sequences. The tree is built according to the distance between LSRs according to the percentage of identical amino acids after alignment. An alignment of related attB sequences, in no particular order, is shown below. At the end of the tree, numbers indicating the attB sequences that are targeted by each LSR are shown. The attB alignment is colored according to consensus sequence similarity, with grey indicating a match to the consensus sequence, four unique colors indicating single nucleotide mismatches from the consensus, and black indicating alignment gaps. FIG. 1F is a schematic of an exemplary technique to identify multi-targeting LSRs. Briefly, if a single cluster of related LSRs (clustered at 90% identity) integrate into multiple diverse target protein families (clustered at 50% identity), then the LSR cluster is considered multi-targeting. The typical domain architecture of a multi-targeting LSR, which includes the addition of a domain of unknown function (yellow; DUF4368), is shown on the right. FIG. 1G is an exemplary observed network of predicted multi-targeting LSRs. Node colors and sizes are the same as in FIG. 1D. FIG. 1H is an alignment of diverse attB sequences that are targeted by a single multi-targeting LSR. Each target sequence is aligned with respect to the core TT dinucleotide. Showing a sequence logo above the alignment to indicate conservation across target sites, implying the sequence specificity of this particular LSR. The alignment is colored according to the consensus, the same as in FIG. 1E.

FIGS. 2A-2N show characterization of new landing pad LSRs. FIG. 2A is a schematic of an exemplary plasmid recombination assay. Cells are co-transfected with LSR-2A-GFP, promoter-less attP-mCherry, and EF1a-attB. Upon recombination, mCherry gains the EF1a promoter and is expressed. FIG. 2B is a plasmid recombination assay of predicted LSRs and att sites in HEK293FT cells. Shown is the fold change of mCherry mean fluorescence intensity (MFI) of all single cells compared to Bxb1. Dots show mean, error bars show standard deviation (n=3 transfection replicates). FIG. 2C is exemplary mCherry distributions for all three plasmids (LSR+attB+attP) compared to the attP-only negative control. Cells are not gated for any transfection delivery markers. FIG. 2D is a plasmid recombination assay between all pairs of LSR+attP and attB in K562 cells (n=1). FIG. 2E is a schematic of an exemplary genomic landing pad assay. An EF1a promoter, attB, and LSR are integrated into the genome of K562 cells via low MOI lentivirus, resulting in a single copy of the landing pad per cell. Clonal cell lines are then electroporated with the attP-mCherry donor plasmid. Upon successful integration into the landing pad, mCherry is expressed, and the LSR and GFP are knocked out. FIG. 2F is flow cytometry of mCherry⁺ cells 11 days after donor electroporation with 1000 ng donor plasmid. Each point is a different clonal K562 cell line carrying the landing pad and LSR corresponding with the donor. Pa01 is significantly more efficient than BxB1 comparing between conditions with donor electroporation (**=P<0.005, one-way ANOVA). FIG. 2G is flow cytometry showing knockout of LSR-GFP and integration of mCherry in the same cells. Pa01 clonal landing pad line was electroporated with donor twice to increase donor delivery, resulting in >70% mCherry⁺ cells. FIG. 2H is flow cytometry of mCherry⁺ cells 18 days after LSR and donor co-electroporation into WT K562 cells that lack a landing pad. attD donor contains its own EF1a promoter and attD donor-only is a negative control. FIG. 2I shows genome-wide integration site mapping by next generation sequencing to measure the percentage of reads found in the genome outside the expected landing pad. Raw (non-unique) reads found at off-targets are shown as a percentage of all reads (*=P<0.05, one-tailed t-test). For Kp03, Ec03, and Pa01, n=2 independent clonal landing pad lines with maximal mCherry 11 days post donor electroporation. For Bxb1, showing two technical replicates of a single clonal landing pad line with maximal mCherry 11 days post donor electroporation. Numbers near the top of each bar indicate (Total number of unique off-target reads)/(Total number of off-target loci). FIG. 2J is a plasmid recombination assay of second batch of predicted LSRs and att sites in HEK293FT cells. Shown is the fold change of mCherry mean fluorescence intensity (MFI) of all single cells compared to Bxb1. Dots show mean, error bars show standard deviation (n=3 transfection replicates). FIG. 2K is exemplary mCherry distributions for three plasmids (LSR+attB+attP), as indicated, compared to the attP-only negative control. Cells were not gated for any transfection delivery markers. FIG. 2L is a graph of the efficiency of promoterless-mCherry donor integration into a polyclonal genomic landing pad (LP) K562 cell lines, measured after 5 days (n=2 independently transduced and then electroporated biological replicates). Asterisks show statistical significance for landing pad plus donor conditions compared to Bxb1 (one-way ANOVA with Dunnett's multiple comparisons est, * is P<0.05, *** is P<0.001, **** is P<0.0001. n.s. is not significant). FIG. 2M shows donor plasmid integration into clonal landing pad cell lines electroporated with 1000 ng donor plasmid (10 days after electroporation, left) or 3000 ng donor plasmid (11 days after electroporation, right). 1000 ng Pa01 is significantly more efficient than 1000 ng Bxb1 comparing between conditions with donor electroporation (P<0.005, one-way ANOVA, n=3 clonal cell lines for Pa01 and n=4 clonal cell lines for others at 1000 ng dose with one electroporation per clone, and n=2 clonal cell lines per LSR at 3000 ng dose with two electroporation replicates per clone, error=s.e.m.). Dots on the left show individual clones, dots on the right show electroporation replicates and each individual clone is separately vertically aligned. FIG. 2N shows representative mCherry distributions for three plasmids (LSR+attB+attP), as indicated, compared to the attP-only negative control.

FIGS. 3A-3K show genome-targeting LSRs can integrate into the human genome at predicted target sites. FIG. 3A is a schematic representation of computational strategy to identify LSRs with innate affinity for the human genome. Briefly, attB/attP candidates in the database were searched against the human genome using BLAST. The attachment site that best matched the human genome would be renamed the attA(cceptor), and the human genome target site would be renamed the attH(uman). The attachment site that did not match the genome would become the attD(onor). FIG. 3B is BLAST hits of attB/P sites that are homologous to sequences in the human genome. Attachment sites for quality-controlled LSR predictions were searched against the human genome using BLAST. Showing all hits that meet E<0.01. Showing four candidates in red that were later shown experimentally to integrate at the predicted target site in the integration site mapping assay. Showing 22 autosomal chromosomes, starting with chromosome 1 in dark blue on the left, and alternating colors with light blue every other chromosome. FIG. 3C is plasmid recombination assay results for LSRs with predicted pseudosites using cognate predicted attachment sites. Candidates shown in red are considered active LSRs with predicted pseudosites (one-tailed t-test, P<0.05), while candidates in grey are candidates with predicted pseudosites that are considered inactive (P>0.05). Highlighting controls and candidates that were validated in the integration site mapping assay. Several of these candidates did not meet quality control filters overall, but were selected due to high similarity between their attachment sites and the human genome. An analysis of how validation rate changes according to candidate quality is shown in FIG. 4A. FIG. 3D shows the BLAST alignments of the microbial attachment sites (attA) to the predicted human attachment sites (attH) for three candidates (SEQ ID NOs: 3494-3499 for attA and attH for Sp56, Pf80, and Enc3, respectively). The attA is shown on the top of each alignment, while the attH is shown on the bottom. FIG. 3E is graphs of the results of integration site mapping experiment to determine true integration at predicted target sites. Integration sites are ranked according to the number of unique reads found at each site. For Sp56 and Pf80, the locus with the most reads corresponded to the predicted locus. For Enc3, the predicted locus was not the most frequently targeted locus, but was still validated as a true integration site. FIG. 3F shows reads that align (in the forward direction (red) and those aligning in the reverse direction (blue), with a black line connected paired reads) to the integration sites for Pf80 in the human genome, showing the predicted target site. FIG. 3G is a graph of human integration assay results of the top candidate from the most recent batch of LSR candidates. While on-target integration was able to be detected for previous genome-targeting candidates, the overall integration efficiency still remains quite low. A new set of predicted genome-targeting candidates, and Dn29 and Vp82 emerged as a top candidates, with 4.5% (+/−0.13%) and 2.52% (+/−0.004%) corrected integration efficiency, respectively. PhiC31 is a previously known genome targeting LSR used as a control, although its efficiency is below the limit of detection (˜1% of cells). Bars are mean, dots are individual transfections. Error=s.d. (*=P<0.05, one-tailed t-test). FIG. 3H shows integration site mapping results for Dn29, and Vp82. Top 3 targeted human genome sites are labeled in each panel. The most commonly targeted site for Dn29 accounts for ˜17% of detected reads, suggesting that this candidate has as favorable mix of efficiency and specificity. FIG. 3I shows target site motif of the top 25 human genome target sites for genome-targeting candidate Dn29. attA sites are SEQ ID NOs: 3500-3503 top to bottom. FIG. 3J shows target site motif of the top 25 human genome target sites for genome-targeting candidate Vp82. attA sites are SEQ ID NOs: 3504-3507 top to bottom. FIG. 3K show LSR integration specificity vs. efficiency. Black points indicate integration into wild-type cells, green points indicate integration into cells with pre-installed landing pads (FIG. 2E). Selected LSRs are labeled. For wild-type cells, efficiency is estimated as percent of mCherry+cells 18 days after electroporation with an LSR and an mCherry expressing donor plasmid corrected by a donor only control transfection. For landing pad cells, efficiency is estimated as the mean of mCherry+cells in all clones of FIG. 2G, right. To estimate specificity, UMI counts were used if available, otherwise uniquely mapped read counts were used, and counts were merged across replicates. FIG. 3L shows the top three integration sites for Dn29, shown in their genomic context. The red line indicates the exact position of integration, with introns and exons of nearby genes in blue.

FIGS. 4A-4G show multi-targeting LSRs are highly efficient and reusable. FIG. 4A is a graph of co-transfection of LSR Cp36 and attD-mCherry donor plasmid to K562 cells without a landing pad. Bxb1 paired with Cp36 attD donor was used as a negative control. The dose in ng refers to the LSR plasmid and the attD donor plasmid was delivered at a 1:1 molar ratio. FIG. 4B is a graph of integration site mapping assay results for Cp36. An integration locus was defined in this experiment as a detected integration of a donor cargo at a specific location. The top 500 loci across two experiments are shown, one performed in HEK293FT cells and another performed in K562 cells. Unique reads result in conservative count estimates for loci with higher coverage. The sequences of sites indicated by arrows are shown at the bottom of FIG. 4C. FIG. 4C is Cp36 target site motifs and example target sequences. Precise integration sites and orientations were inferred at all loci, and nucleotide composition was calculated for the top 200 sites in the HEK293FT and K562 experiments. The core dinucleotide is found at the center. Example integration sites are shown below, colored according to nucleotides (SEQ ID NOs 3508-3512). FIG. 4D is a graph of efficiency of Cp36 vs. PiggyBac (PB) for stable delivery of mCherry donor plasmid in K562 cells, 10 days post-transfection. The donor plasmid contains both the Cp36 attD and the PiggyBac ITRs and Ec03 LSR is used as a negative control that lacks an attachment site on this donor plasmid. FIG. 4E is a graph of mCherry integration efficiency of Cp36, with and without redosing with Cp36 at day 15. FIG. 4F is a graph of wild-type K562 or Cp36-dosed mCherry+ and puromycin-selected cells transfected with a second fluorescent reporter (mTagBFP2) and analyzed by flow cytometry 13 days post-electroporation with 2000 ng of BFP donor and an equimolar dose, 1600 ng, of Cp36 plasmid. Bars show the mean, dots show replicates, error=s.e.m. (n=2 electroporation replicates). Dash shows negative control treated with BFP donor only. Corresponding mCherry levels are shown in FIG. 11D. FIG. 4G is flow cytometry analysis 12 days post-electroporation of both fluorescent donors and Cp36 plasmids into K562 cells. Negative control cells were transfected with the donors and pUC19. Error=s.e.m. (n=2 electroporation replicates).

FIG. 5A is a phylogenetic tree of 1081 LSR clusters (50% identity) identified. Tips are colored according to the phylum of bacterial host species. First heat map ring is colored according to the number of unique target gene clusters that each LSR cluster is predicted to integrate into, the same as in FIG. 1B. The second ring of green annotations indicate LSR clusters that are predicted to contain the DUF4368 Pfam domain. Clusters for controls Bxb1 and PhiC31 are indicated in bold text, and clusters for select candidates with experimental validation are also indicated. FIG. 5B shows the Pfam domains that are most commonly found in target genes. Each target gene was annotated using Pfam HMM models, and then the total number of LSR clusters that integrate into genes containing each Pfam domain was calculated. FIG. 5C shows an alignment of LSR sequences that are presented in FIG. 1E. Resolvase, Recombinase, and Zn_recomb_ribbon Pfam domains are indicated. Above each aligned amino acid position, the height and color of each bar indicates the mean pairwise identity over all pairs in the column, with green indicating 100% identity across all sequences, green-brown indicating above 30% identity and below 100% identity, and red indicating below 30% identity. FIG. 5D shows exemplary predicted attB motifs. Each column represents a different LSR attB motif. The first row shows motifs that were derived from different attB sequences that were all targeted by a single, unique LSR protein. The second row shows motifs that were derived from attB sequences that were targeted by LSR proteins that fell into a single 90% identity cluster. The third row shows motifs that were derived from attB sequences that were targeted by LSR proteins that fell into a single 50% identity cluster. FIG. 5E is Pfam domain enrichment analysis of target genes. Pfam domains that reach a significance cutoff of FDR<0.05 are shown. Pfam domains are ordered and displayed according to the −log 10(P) value of a Fisher's exact test. Numbers next to each point indicate the total number of target gene clusters that contain the specified domain. FIG. 5F is gene ontology (GO) term enrichment analysis of target genes. All 6 terms that reach a significance cutoff of FDR<0.1 are shown. Terms are ordered and displayed according to the −log 10(P) value of a Fisher's exact test. Numbers next to each point indicate the total number of target gene clusters that fall under the specified GO term. FIG. 5G shows distances between target genes and the nearest phage defense gene. For each target gene that appears on a contiguous sequence with a defense gene, the distance is calculated, and then a random gene from the same contiguous sequence is selected as a background control. Showing boxplot with median, 1st and 3rd quartiles, 1.5×IQR as whiskers, and outliers as points. Wilcoxon rank-sum test used to test for significant differences between groups.

FIGS. 6A-6O show characterization of landing pad LSRs. FIG. 6A is a graph of the efficiency of promoterless-mCherry donor integration into a genomic landing pad (LP) in K562 cells measured by flow cytometry. Landing pad and donor are the same constructs shown in FIG. 2E, but here polyclonal landing pad lines were derived by high MOI delivery of the lentiviral landing pad without any subsequent selection or sorting. 1.2 million K562 cells were electroporated with 600 ng donor plasmids with attP corresponding to the LSR and measured after 5 days (n=2 independently transduced and then electroporated biological replicates). Asterisks show statistical significance for landing pad plus donor conditions compared to BxB1 (one-way ANOVA with Dunnett's multiple comparisons test, * is P<0.05, *** is P<0.001, **** is P<0.0001, n.s. is not significant). FIG. 6B is a graph of the stability of polyclonal landing pads expressing LSR-GFP as measured by flow cytometry over time. These cells are not electroporated with donor and day 5 was the same day of measurement as for FIG. 6D (n=2 independently transduced biological replicates). FIG. 6C is flow cytometry measuring mCherry⁺ cells 10 days after electroporation with 2000 ng donor plasmid. Each point is a different clonal K562 cell line carrying the landing pad and LSR corresponding with the donor. Error bar shows standard deviation for conditions with multiple clones. FIG. 6D is flow cytometry measuring mCherry⁺ cells 12 days after electroporation with 2000 or 5000 ng donor plasmid into clonal K562 cell lines carrying the landing pad. Error bar shows standard deviation (n=3 electroporation replicates shown as dots). FIG. 6E shows the minimization of Pa01 attB sequence by trimming nucleotides from either end and using the plasmid recombination assay. Arrows indicate shortest attB which did not disrupt recombination activity. The inferred 33 bp minimal attB as determined by this experiment is shown between vertical lines at the bottom within SEQ ID No: 3513 shown. Colored rectangles show mean corrected mCherry MFI (n=3 transfection replicates in HEK293FT cells). The attB in the top rectangle extends in both directions and is the full length attB as retrieved from the LSR database and used in FIGS. 2B-2C. FIG. 6F shows minimization of Kp03 attB sequence by trimming nucleotides from both ends using the plasmid recombination assay. The shortest tested attB was 25 nucleotides. Colored rectangles show mean mCherry MFI normalized to attD only MFI (n=3). The attB in the top rectangle extends in both directions and is the full length attB as retrieved from the LSR database and used in FIGS. 2B-2C. The dinucleotide core, as determined by off-target integration site mapping, is shown in bold text within SEQ ID No: 3514 shown. FIG. 6G is a graph of Kp03 dinucleotide core swapping in plasmid recombination assay to determine the capacity to program specific matches between donors and acceptor attachment sites by changing the core. AC is the native dinucleotide core sequence. Values are mean±SD with n=3 transfection replicates in HEK293FT cells. FIG. 6H is a target site motif of the top 25 human genome target sites for landing pad candidates Kp03 (top) and Pa01 (bottom). Core dinucleotides are strongly conserved among integration sites for both candidates. FIG. 6I is a schematic of optimized integration site mapping assay, a modified version of UdiTaS. Addition of a round of amplification using a nested donor primer is expected to enrich for desired target-derived reads, which includes both donor-only reads and donor-genome junction reads. FIG. 6J is a graph of the proportion of reads derived from different sources in the integration site mapping assay. On the left, the proportions before assay optimization, and after optimization on the right. Both runs are of Cp36 circular donor experiments, but in two different cell types (HEK293FT on the left, K562 on the right). Target-derived reads are those that come from the donor only (light green) or the donor-genome integration junction reads (dark green). FIG. 6K is flow cytometry measuring mCherry⁺ cells 18 days after LSR and donor co-electroporation into WT K562 cells that lack a landing pad. attD donor contains its own EF1a promoter and attD donor-only is a negative control. FIG. 6L shows the results from a plasmid recombination assay of predicted LSRs and att sites in HEK293FT cells, as percentage of mCherry+cells gated on GFP positive cells. mCherry and GFP gating is determined based on an empty backbone transfection. Dots show each transfection replicate, error=s.d. (n=3 transfection replicates). FIG. 6M is a graph of the fraction GFP+cells in clonal cell lines 27 days after transduction. GFP+cells were sorted into wells as single cells to generate clonal lines, expanded for two weeks, measured by flow cytometry, and graded as GFP+ if the population was >95% GFP+, suggesting a lack of transcriptional silencing. Sixteen wells were sorted for each LSR, and the number of wells with a live cell population at the time of flow analysis is shown in the legend. For all LSRs, some wells were empty, possibly due to a sorting miss or cell death. FIG. 6N is a graph of flow cytometry measuring mCherry+cells 18 days after LSR and donor co-electroporation into WT K562 cells that lack a landing pad. attD donor contains an EF-1α promoter driving mCherry expression and attD donor transfected with a non-matching LSR is a negative control (*=P<0.05, **=P<0.005, one-tailed t-test) (error=s.d. n=2 transfection replicates). FIG. 6O shows genome-wide integration site mapping by next generation sequencing to measure the percentage of reads found in the genome outside the expected landing pad. For Kp03. Ec03, n=2 independent clonal landing pad lines were used, and for Pa01 n=3 clonal landing pad lines were used, with maximal mCherry 11 days post donor electroporation. For Bxb1, three technical replicates (starting from different gDNA aliquots) of a single clonal landing pad line with maximal mCherry 11 days post donor electroporation are shown. Raw (non-unique) reads found at off-targets as a percentage of all reads are shown (*=P<0.05, one-tailed t-test). Numbers near the top of each bar indicate the total number of off-target loci on the left, and below in parentheses are the subset of those sites that replicate in landing pad cell lines (left) and the subset that replicate in wild-type cell lines (right).

FIGS. 7A-7F show characterization of genome-targeting. FIG. 7A is a graph of the proportion of LSRs that mediate significant recombination in the plasmid recombination assay with and without application of quality control (QC) thresholds for LSR candidate selection. The numbers above each bar indicate the (number of candidates that met P<0.05 in the plasmid recombination assay)/(total number of tested candidates). FIG. 7B is a graph of a plasmid recombination assay for top genome-targeting candidates using predicted attH sites. FIGS. 7C and 7D show reads that align (in the forward direction (red) and those aligning in the reverse direction (blue), with a black line connected paired reads) to the integration sites for Sp56 and Enc3, respectively, in the human genome. The orientation and location of the integration changes when using a linear donor, whereas the exact predicted integration site is targeted with a circular donor. FIGS. 7E and 7F show the target site motifs for Dn29 and Vp82, respectively. On each row, motifs are shown with different subsets of the integration sites.

FIG. 8A are graphs of Cp36 mCherry donor cargo integration in K562 cells without pre-installation of a landing pad or antibiotic selection utilizing both plasmid DNA and linear PCR amplicons as the donor cargo. FIG. 8B is a graph of additional multi-targeting LSRs validated using the pseudosite integration assay. Showing two additional candidates, Pc01 and Enc9, which are both found in the multi-targeting clade. FIG. 8C is a schematic of the integration sites found for Cp36 using the integration site mapping assay. FIG. 8D is a schematic of a plasmid recombination assay with swapped att sites and the results for Cp36 compared with multiple landing pad LSRs. FIG. 8E is a schematic of an exemplary plasmid used for direct comparison of Cp36 and PiggyBac containing both the PB inverted terminal repeats (ITRs) and the Cp36 attD.

FIG. 9 is a schematic of the canonical (can.) LSR integration mechanism. Briefly, an LSR protein (composed of three distinct domains and a coiled coil structural motif) recognizes an attP sequence of nucleotides on a donor plasmid and an attB sequence on a target genome. Four LSR monomers come together to catalyze recombination between the two attachment sites. This results in a unidirectional reaction that forms the final integrated product.

FIG. 10 shows a phylogenetic tree of identified LSRs with phylogenetic clades, which include 2 or more experimentally active LSRs which descend from a common ancestor.

FIGS. 11A-11F show multi-targeting recombinases are efficient and unidirectional integrases. FIG. 11A shows the correlation between read counts from the Cp36 integration site mapping assay across HEK293FT and K562 cell lines. The top 61 shared loci, all of which are found among the top 200 most frequently targeted sites in the two cell types are shown. The gray band indicates the 95% confidence interval. FIG. 11B shows enrichment of target sites in DNase hypersensitivity peaks for several multi-targeters. Fisher's exact test was used to calculate statistical significance of each enrichment. P-values and number of relevant integration sites are shown above each relevant lane. Error bars indicate the 95% confidence interval. FIG. 11C shows target site motif as predicted using 33 attB sequences in the LSR-attachment site database that are targeted by LSRs that fall in the same 50% amino acid identity cluster as Cp36. Method used to construct this motif is the same as in FIGS. 1H and 5G. Schematic on the left of FIG. 11D depicts a Cp36 re-dosing experiment wherein Cp36 and an mCherry donor are used to generate mCherry+cells, and then Cp36 enzyme or the empty LSR expression backbone is re-dosed, followed by flow cytometry to measure possible excision of the mCherry cargo. FIG. 11D on the right, shows the mean percentage of mCherry+cells on day 18 as measured by flow cytometry (n=2 transfection replicates). FIG. 11E shows delivery of the BFP donor alone. K562 cells were electroporated with 2400 ng of Cp36 plasmid and 3000 ng of BFP donor plasmid and BFP was measured by flow cytometry after 12 days. Dash refers to unelectroporated cells, and the Cp36- or donor-only conditions include pUC19 stuffer plasmid so the mass delivered is equal. Bars show mean, dots show replicates. FIG. 11F shows Cp36-dosed mCherry+ and puromycin-selected cells analyzed by flow cytometry 13 days postelectroporation with 2000 ng of BFP donor and an equimolar dose. 1600 ng, of Cp36 plasmid (or pUC19 stuffer plasmid). Bars show the mean, dots show replicates (error=s.e.m. n=2 electroporation replicates). Dash shows unelectroporated control.

FIGS. 12A-12C show post hoc identification of human genome integration sites using database sequence motifs. FIG. 12A shows the performance of database-derived sequence motifs to predict human genome integration sites as measured by ROC curve analysis. Sequence motifs for each LSR were automatically generated from the bacterial sequence database by selecting non-redundant (95% nucleotide identity) attB sequences of related LSR orthologs. These motifs were then searched against true integration sites and randomly selected background sequences using the HOMER motif analysis software. ROC curves were generated by sliding across a relevant range of motif score cutoffs and calculating the false positive rate (x-axis) and true positive rate (y-axis) at each cutoff. The area under the curve (AUC) was then calculated as a single measure of predictive performance. Each ROC curve is labeled with the relevant LSR name and the number of integration sites detected across all relevant experiments. FIG. 12B shows distributions of normalized HOMER motif scores in experimentally observed integration sites (“Obs.”) vs. randomly selected background sequences (“Rand.”). Showing boxplot with median, 1st and 3rd quartiles, 1.5×IQR as whiskers, and outliers as points. One-sided Wilcoxon rank-sum test used to test for significant differences between groups (** is P<0.01, **** is P<0.0001, n.s. is not significant). Red points indicate the normalized HOMER motif score for the observed integration site with the most experimentally detected integration events relative to all other integration sites for each LSR. FIG. 11C shows the final sequence motifs used to predict human genome integration sites for each LSR. Each sequence is labeled with the relevant LSR, the number of attB sequences used to build the motif, and the mean percentage amino acid identity of all the LSR orthologs that were used to identify related attB sequences.

### DETAILED DESCRIPTION

Described herein are large serine recombinases (LSRs) identified along with their cognate DNA attachment sites using a computational workflow. The LSRs were characterized according to three separate technological applications: 1) landing-pad LSRs that can integrate efficiently at a pre-installed integration site, 2) multi-targeting LSRs that can integrate efficiently at many different loci in a target genome, and 3) genome-targeting LSRs that can integrate at one or several specific target sites in a given target genome. Several candidates in all three of these categories were validated in human cells. For landing-pad LSRs, many candidates were identified that recombined at orthogonal attachment sites at high efficiency when compared to Bxb1, the existing gold standard. For multi-targeting LSRs, which have not previously been developed as an integration tool in human cells, several were identified that can integrate at high efficiency in human cell lines relative to ΦC31. For genome-targeting LSRs, several candidates that integrate DNA cargos into predicted human genome target sites without pre-installation of an attachment site were identified and validated.

Recombinases have vast applications as genome engineering tools. However, efficient genome integration of large donor sequences into the human genome is an outstanding problem in the field of human genome engineering. One major hurdle is the cargo size limit of adeno-associated virus (AAV) vector, the most successful vector available for human genome engineering, which is around 4.7 kilobase pairs (kb). CRISPR-Cas9 can be used to introduce double-stranded breaks at programmable locations, but when followed by homologous recombination to introduce new DNA, the efficiency of integration decreases exponentially as the size of the insertion increases, with reported maximum insertion sizes of 3-6 kb. By contrast, for recombinases, there is no obvious upper limit on the size of the donor DNA to be integrated, which is a major advantage of recombinases over other technologies.

Section headings as used in this section and the entire disclosure herein are merely for organizational purposes and are not intended to be limiting.

### 1. DEFINITIONS

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not. As used herein, comprising a certain sequence or a certain SEQ ID NO usually implies that at least one copy of said sequence is present in recited peptide or polynucleotide. However, two or more copies are also contemplated.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002)) and U.S. Pat. No. 5,034,506), locked nucleic acid (LNA: see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

A “peptide” or “polypeptide” is a linked sequence of two or more amino acids linked by peptide bonds. The peptide or polypeptide can be natural, synthetic, or a modification or combination of natural and synthetic. Polypeptides include proteins such as binding proteins, receptors, and antibodies. The proteins may be modified by the addition of sugars, lipids or other moieties not included in the amino acid chain. The terms “polypeptide” and “protein,” are used interchangeably herein.

As used herein, the term “percent sequence identity” refers to the percentage of nucleotides or nucleotide analogs in a nucleic acid sequence, or amino acids in an amino acid sequence, that is identical with the corresponding nucleotides or amino acids in a reference sequence after aligning the two sequences and introducing gaps, if necessary, to achieve the maximum percent identity. Hence, in case a nucleic acid according to the technology is longer than a reference sequence, additional nucleotides in the nucleic acid, that do not align with the reference sequence, are not taken into account for determining sequence identity. A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3×, FAS™, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)).

The term “amino acid” or “any amino acid” as used here refers to any and all amino acids, including naturally occurring amino acids (e.g., a-amino acids), unnatural amino acids, modified amino acids, and non-natural amino acids. It includes both D- and L-amino acids. Natural amino acids include those found in nature, such as, e.g., the 23 amino acids that combine into peptide chains to form the building-blocks of a vast array of proteins. These are primarily L stereoisomers, although a few D-amino acids occur in bacterial envelopes and some antibiotics. The “non-standard,” natural amino acids include, for example, pyrolysine (found in methanogenic organisms and other eukaryotes), selenocysteine (present in many non-eukaryotes as well as most eukaryotes), and N-formylmethionine (encoded by the start codon AUG in bacteria, mitochondria, and chloroplasts). “Unnatural” or “non-natural” amino acids are non-proteinogenic amino acids (e.g., those not naturally encoded or found in the genetic code) that either occur naturally or are chemically synthesized. Over 140 unnatural amino acids are known and thousands of more combinations are possible. Examples of “unnatural” amino acids include β-amino acids (β³ and β²), homo-amino acids, proline and pyruvic acid derivatives, 3-substituted alanine derivatives, glycine derivatives, ring-substituted phenylalanine and tyrosine derivatives, linear core amino acids, diamino acids, D-amino acids, alpha-methyl amino acids and N-methyl amino acids. Unnatural or non-natural amino acids also include modified amino acids. “Modified” amino acids include amino acids (e.g., natural amino acids) that have been chemically modified to include a group, groups, or chemical moiety not naturally present on the amino acid.

For the most part, the names of naturally occurring and non-naturally occurring aminoacyl residues used herein follow the naming conventions suggested by the IUPAC Commission on the Nomenclature of Organic Chemistry and the IUPAC-IUB Commission on Biochemical Nomenclature as set out in “Nomenclature of α-Amino Acids (Recommendations, 1974)” Biochemistry, 14(2), (1975). To the extent that the names and abbreviations of amino acids and aminoacyl residues employed in this specification and appended claims differ from those suggestions, they will be made clear.

Throughout the present specification, unless naturally occurring amino acids are referred to by their full name (e.g., alanine, arginine, etc.), they are designated by their conventional three-letter or single-letter abbreviations (e.g., Ala or A for alanine, Arg or R for arginine, etc.). The term “L-amino acid,” as used herein, refers to the “L” isomeric form of a peptide, and conversely the term “D-amino acid” refers to the “D” isomeric form of a peptide (e.g., Dphe, (D)Phe, D-Phe, or ^{D}F for the D isomeric form of Phenylalanine). Amino acid residues in the D isomeric form can be substituted for any L-amino acid residue, as long as the desired function is retained by the peptide.

In the case of less common or non-naturally occurring amino acids, unless they are referred to by their full name (e.g. sarcosine, ornithine, etc.), frequently employed three- or four-character codes are employed for residues thereof, including, Sar or Sarc (sarcosine, i.e. N-methylglycine), Aib (α-aminoisobutyric acid), Dab (2,4-diaminobutanoic acid). Dapa (2,3-diaminopropanoic acid), γ-Glu (γ-glutamic acid), Gaba (γ-aminobutanoic acid), β-Pro (pyrrolidine-3-carboxylic acid), and 8Ado (8-amino-3,6-dioxaoctanoic acid), Abu (2-amino butyric acid), βhPro (β-homoproline), βhPhe (β-homophenylalanine) and Bip (β,βdiphenylalanine), and Ida (Iminodiacetic acid).

The term “pharmaceutically acceptable salt” in the context of the present invention

The terms “non-naturally occurring,” “engineered,” and “synthetic” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

A “vector” or “expression vector” is a replicon, such as plasmid, phage, virus, or cosmid, to which another DNA segment, e.g., an “insert,” may be attached or incorporated so as to bring about the replication of the attached segment in a cell.

A cell has been “genetically modified,” “transformed,” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

The term “contacting” as used herein refers to bring or put in contact, to be in or come into contact. The term “contact” as used herein refers to a state or condition of touching or of immediate or local proximity. Contacting a system to a target destination, such as, but not limited to, an organ, tissue, cell, or tumor, may occur by any means of administration known to the skilled artisan.

As used herein, the terms “providing.” “administering.” “introducing,” are used interchangeably herein and refer to the placement of the systems, recombinases, or nucleic acids of the disclosure into a cell, organism, or subject by a method or route which results in at least partial localization of the system to a desired site. The systems, recombinases, or nucleic acids can be administered by any appropriate route which results in delivery to a desired location in the cell, organism, or subject.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species: farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods and compositions provided herein, the mammal is a human.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. RECOMBINASE SYSTEMS

The present disclosure provides systems for DNA modification comprising: a polypeptide comprising a recombinase (e.g., a large serine recombinase) having an amino acid sequence having at least 70% identity (e.g., at least 75%, at least 80%, at least 85%, at least 90%, at least 95%, at least 97%, at least 98%, at least 99%, or 100%) to any of SEQ ID NOs: 1-74, or a nucleic acid encoding thereof; and a first polynucleotide comprising a donor recognition sequence for the recombinase. Also provided herein are enzymatically active fragments thereof (e.g., C- or N-terminal truncations or containing internal deletions, but retaining the desired enzymatic activity). The active fragment may contain at least 20 amino acids, at least 30 amino acids, at least 40 amino acids, at least 50 amino acids, at least 100 amino acids, or more of SEQ ID NOs: 1-74 or sequences at least 70% identity to at least 20 amino acids, at least 30 amino acids, at least 40 amino acids, at least 50 amino acids, at least 100 amino acids, or more of SEQ ID NOs: 1-74. In some embodiments, the recombinase has an amino acid sequence having at least 70% (e.g., at least 75%, at least 80%, at least 85%, at least 90%, at least 95%, at least 97%, at least 98%, at least 99%, or 100%) identity to any of SEQ ID NOs: 2, 6, 10, 12, 18, 19, 26, 29, 61, 65, or 66, or an active fragment thereof. In select embodiments, the recombinase has an amino acid sequence of SEQ ID NOs: 2, 6, 10, 12, 18, 19, 26, 29, 61, 65, or 66, or an active fragment thereof.

The present disclosure also provides systems for DNA modification comprising: a polypeptide comprising a recombinase (e.g., a large serine recombinase), or a nucleic acid encoding thereof; and a first polynucleotide comprising a donor recognition sequence for the recombinase, wherein the recombinase (e.g., a large serine recombinase) comprises one or more of the following amino acid motifs, written in the common Prosite format, where the potential amino acids at any one position are in square brackets, x is any amino acid and x(n) represents n number of any amino acid (e.g., x(3) is xxx or 3 consecutive amino acids):

Motif 1:
[AEILSTVY]-[ADEGKQRST]-x(3)-[EG]-x-[ACFLMV]-x-[AFILMTV]-x(2)-[FHILMNV]-
[AGSV]-[ADILSTV]-x-[AGS]-x(3)-[KRSV]-[ADEGKNST]-[AEIKMNQST]-[FILMST]-x-
[DELQSV]-[ENQR]-x(4)-[AFHIKLMNQRSV]-x-[AEGHKLMNQRSV]
Motif 2:
[AGI]-[DEGNPSTV]-[DGNQS]-[AHNQRTVY]-x-[ADEHILPQRTY]-[ADEQR]-[FIKL]-x-
[DEFGNQRSTV]-[AILSTV]-[DEIKLNQRSTV]-[ADEKMNRSTV]-[AGQRST]-x-
[ADEKLQRT]-x-[ALMV]
Motif 3:
[ADFILMNSY]-x(2)-[AIKMSV]-x-[AFGILMV]-x(3)-[QRT]-[AGS]-x-[DEGNQS]-E-S-x-
[AHKNRSTV]-K-x(2)-[LMRY]-[AINQSTV]-[AEFIKLNRTV]-x-[AFHLNQSTY]-
[AILMNRSTVY]
Motif 4:
[EKNTGSLDVARP]-[EHITGSLDVAP]-x-[MITSLVARP]-[EKNITGSDQVARP]-
[EGSDARP]-[ILDAR]-[MHKTLVQDAR]-[EKITGSLDQVA]-[EKHDQVAR]-
[MHISLVQAR]-[QEKNMSLDVAR]-[EKHGSLDQAR]-[EYKNIHLVA]-x-
[EKITGSLDQAR]-[EKHTGDQAR]-x-[QEKNTGSDVAR]-[QEKNTGSVDAR]-
[SWLVFAR]-[QEMTGSLVDA]-[EKNITGSDARP]-[EMILDQA]-[EYILVFAR]-
[EMTGSLDVAR]-[EKNGSLDQAR]-[QEGVDARP]
Motif 5:
[ADEHKNQRS]-[ADEFGHKMNQRSWY]-[EFY]-[FHLWY]-x-[ADEFIKLMNQRSTY]-
[FIQSTV]-[AGKLNRSTV]-[ADEHKNQRTY]-[INQR]-[FILMQS]-x(2)-[AGKNS]-
[KMQRSTV]-x(2)-[AEGKMNSTY]
Motif 6:
W-[AEHNRSTV]-x-[AGNST]-[FGLMNQSTV]-[ILPV]-x(2)-[ILTV]-x(4)-[ACGMQRST]-x-
[ILVY]-G-[DEHNQS]-x-[EHILMQRT]-[AEFHLNPY]-[CFHKMNQRTY]-[DEFIKLNQRSTV]
Motif 7:
[AGINSTV]-x-[AIS]-x-[FILMY]-E-[IR]-x(2)-[DILT]-x-[AEIKMQS]-R-[ITV]-x-
[ADGRST]-x-[FKLMY]-[AEHIKLMNQRVWY]-x-[AIKLMR]
Motif 8:
[FY]-[DEKQS]-[EKLMQ]-[KLR]-[KLV]-x-[GN]-[DEHKLMR]-[ST]-x-[FHIQSTVW]
Motif 9:
[ILV]-x(2)-[ADFHILMNQSVY]-x(3)-[AGS]-x-[DEIKNQRS]-[EQ]-S-x(2)-[AK]-[AQRS]-
x-[LMR]-[ILQRSV]-x-[ADEGHIQRS]-[AKNQSTV]-[AHKRWY]-x-[AGHIKQRST]-x-
[CHIKLRV]
Motif 10:
R-[LMQR]-[ANS]-[NPST]-W
Motif 11:
[LV]-[AV]-x-[AFHILQWY]-[IMV]-x-[ELQT]-[AIV]-F
Motif 12:
R-[DKNRSV]-[ADEFGKPQS]-[AEIKLSTV]-x-[FGILNV]-[AFILQRVY]-[DEILMNQSTV]-
[DEFILMQTVY]-[IKLRV]-[DEKNQR]-[DEFKLNQWY]-[FL]
Motif 13:
[AEFILMNQSTVY]-[AFGILMRSTV]-x(3)-[ADEFGHLMNST]-x(2)-[DMNS]-[DEQ]-x-
[CFHLTVY]-x-[AEKLRY]-x(2)-[ALS]-x-[DEKNQRS]-[GIMQRTV]-[DHKNQR]-x-
[AGILNSTV]-[FHIKLMNQVWY]

Alternatively, the motifs can be written as the following, where each position is defined by a designated amino acid or X, wherein Xis the amino acid options in brackets, or any amino acid, as indicated.

### Motif 1:

X₁ₐX₂ₐX₃ₐX₄ₐX₅ₐX₆ₐX₇ₐX₈ₐX₉ₐX₁₀ₐX₁₁ₐX₁₂ₐX₁₃ₐX₁₄ₐX₁₅ₐX₁₆ₐX₁₇ₐX₁₈ₐX₁₉ₐX₂₀ₐX₂₁ₐX₂₂ₐX₂₃ₐX₂₄ₐX₂₅ₐX₂₆ₐX₂₇ₐX₂₈ₐX₂₉ₐX₃₀ₐX₃₁ₐX₃₂ₐX₃₃ₐX₃₄ₐ, wherein:

X₃ₐ, X₄ₐ, X₅ₐ, X₇ₐ, X₉ₐ, X₁₁ₐ, X₁₂ₐ, X₁₆ₐ, X₁₈ₐ, X₁₉ₐ, X₂₀ₐ, X₂₅ₐ, X₂₈ₐ, X₂₉ₐ, X₃₀ₐ, X₃₁ₐ, and X₃₃ₐ are each individually selected from any amino acid;

X₁ₐ is A, E, I, L, S, T, V, or Y;

X₂ₐ is A, D, E, G, K, Q, R, S, or T;

X₆ₐ is E or G;

X₈ₐ is A, C, F, L, M, or V;

X₁₀ₐ is A, F, I, L, M, T, or V;

X₁₃ₐ is F, H, I, L, M, N, or V;

X₁₄ₐ is A, G, S, or V;

X₁₅ₐ is A, D, I, L, S, T, or V;

X₁₇ₐ is A, G, or S;

X₂₁ₐ is K, R, S, or V;

X₂₂ₐ is A, D, E, G, K, N, S, or T;

X₂₃ₐ is A, E, I, K, M, N, Q, S, or T;

X₂₄ₐ is F, I, L, M, S, or T;

X₂₆ₐ is D, E, L, Q, S, or V;

X₂₇ₐ is E, N, Q, or R;

X₃₂ₐ is A, F, H, I, K, L, M, N, Q, R, S, or V

X₃₄ₐ is A, E, G, H, K, L, M, N, Q, R, S, or V

### Motif 2:

X_{1b}X_{2b}X_{3b}X_{4b}X_{5b}X_{6b}X_{7b}X_{8b}X_{9b}X_{10b}X_{11b}X_{12b}X_{13b}X_{14b}X_{15b}X_{16b}X_{17b}X_{18b}, wherein

X_{5b}, X_{9b}, X_{15b}, and X_{17b} are each individually selected from any amino acid;

X_{1b} is A, G, or I;

X_{2b} is D, E, G, N, P, S, T, or V;

X_{3b} is D, G, N, Q, or S;

X_{4b} is A, H, N, Q, R, T, V, or Y;

X_{6b} is A, D, E, H, I, L, P, Q, R, T, or Y;

X_{7b} is A, D, E, Q, or R;

X_{8b} is F, I, K, or L;

X_{10b} is D, E, F, G, N, Q, R, S, T, or V;

X_{11b} is A, I, L, S, T, or V;

X_{12b} is D, E, I, K, L, N, Q, R, S, T, or V;

X_{13b} is A, D, E, K, M, N, R, S, T, or V;

X_{14b} is A, G, Q, R, S, or T;

X_{16b} is A, D, E, K, L, Q, R, or T; and

X_{18b} is A, L, M, or V

### Motif 3:

X_{1c}X_{2c}X_{3c}X_{4c}X_{5c}X_{6c}X_{7c}X_{8c}X_{9c}X_{10c}X_{11c}X_{12c}X_{13c}ESX_{16c}X_{17c}KX_{19c}X_{20c}X_{21c}X_{22c}X_{23c}X_{24c}X_{25c}X_{26c}, wherein

X_{2c}, X_{3c}, X_{5c}, X_{7c}, X_{8c}, X_{9c}, X_{12c}, X_{16c}, X_{19c}, X_{20c}, and X_{24c} are each individually selected from any amino acid;

X_{1c} is A, D, F, I, L, M, N, S, or Y;

X_{4c} is A, I, K, M, S, or V;

X_{6c} is A, F, G, I, L, M, or V;

X_{10c} is Q, R, or T;

X_{11c} is A, G, or S;

X_{13c} is D, E, G, N, Q, or S;

X_{17c} is A, H, K, N, R, S, T, or V;

X_{21c} is L, M, R, or Y;

X_{22c} is A, I, N, Q, S, T, or V;

X_{23c} is A, E, F, I, K, L, N, R, T, or V;

X_{25c} is A, F, H, L, N, Q, S, T, or Y;

X_{26c} is A, I, L, M, N, R, S, T, V, or Y

### Motif 4:

X_{1d}X_{2d}X_{3d}X_{4d}X_{5d}X_{6d}X_{7d}X_{8d}X_{9d}X_{10d}X_{11d}X_{12d}X_{13d}X_{14d}X_{15d}X_{16d}X_{17d}X_{18d}X_{19d}X_{20d}X_{21d}X_{22d}X_{23d}X_{24d}X_{25d}X_{26d}X_{27d}X_{28d}, wherein:

X_{3d}, X_{15d}, and X_{18d} are each individually selected from any amino acid;

X_{1d} is E, K, N, T, G, S, L, D, V, A, R, or P;

X_{2d} is E, H, I, T, G, S, L, D, V, A, or P;

X_{4d} is M, I, T, S, L, V, A, R or P;

X_{5d} is E, K, N, I, T, G, S, D, Q, V, A, R, or P;

X_{6d} is E, G, S, D, A, R, or P;

X_{7d} is I, L, D, A, or R;

X_{8d} is M, H, K, T, L, V, Q, D, A, or R;

X_{9d} is E, K, I, T, G, S, L, D, Q, V, or A;

X_{10d} is E, K, H, D, Q, V, A, or R;

X_{11d} is M, H, I, S, L, V, Q, A, or R;

X_{12d} is Q, E, K, N, M, S, L, D, V, A, or R;

X_{13d} is E, K, H, G, S, L, D, Q, A, or R;

X_{14d} is E, Y, K, N, I, H, L, V, or A;

X_{16d} is E, K, I, T, G, S, L, D, Q, A, or R;

X_{17d} is E, K, H, T, G, D, Q, A, or R:

X_{19d} is Q, E, K, N, T, G, S, D, V, A, or R;

X_{20d} is Q, E, K, N, T, G, S, V, D, A, or R;

X_{21d} is I, S, W, L, V, F, A, or R;

X_{22d} is Q, E, M, T, G, S, L, V, D, or A;

X_{23d} is E, K, N, I, T, G, S, D, A, R, or P;

X_{24d} is E, M, I, L, D, Q, or A:

X_{25d} is E, Y, I, L, V, F, A, or R;

X_{26d} is E, M, T, G, S, L, D, V, A, or R;

X_{27d} is E, K, N, G, S, L, D, Q, A, or R;

X_{28d} is Q, E, G, V, D, A, R, or P; and

### Motif 5:

X₁ₑX₂ₑX₃ₑX₄ₑX₅ₑX₆ₑX₇ₑX₈ₑX₉ₑX₁₀ₑX₁₁ₑX₁₂ₑX₁₃ₑX₁₄ₑX₁₅ₑX₁₆ₑX₁₇ₑX₁₈ₑ, wherein:

X₅ₑ, X₁₂ₑ, X₁₃ₑ, X₁₆ₑ, and X₁₇ₑ are each individually selected from any amino acid;

X₁ₑ is A, D, E, H, K, N, Q, R, or S;

X₂ₑ is A, D, E, F, G, H, K, M, N, Q, R, S, W, or Y;

X₃ₑ is E, F, or Y;

X₄ₑ is F, H, L, W, or Y;

X₆ₑ is A, D, E, F, I, K, L, M, N, Q, R, S, T, or Y;

X₇ₑ is F, I, Q, S, T, or V;

X₈ₑ is A, G, K, L, N, R, S, T, or V;

X₉ₑ is A, D, E, H, K, N, Q, R, T, or Y;

X₁₀ₑ is I, N, Q, or R;

X₁₁ₑ is F, I, L, M, Q, or S;

X₁₄ₑ is A, G, K, N, or S;

X₁₅ₑ is K, M, Q, R, S, T, or V;

X₁₈ₑ is A, E, G, K, M, N, S, T, or Y;

### Motif 6:

WX_{2f}X_{3f}X_{4f}X_{5f}X_{6f}X_{7f}X_{8f}X_{9f}X_{10f}X_{11f}X_{12f}X_{13f}X_{14f}X_{15f}X_{16f}GX_{18f}X_{19f}X_{20f}X_{21f}X_{22f}X_{23f}, wherein:

X_{3f}, X_{7f}, X_{8f} X_{10f}, X_{11f}, X_{12f}, X_{13f}, X_{15f}, and X_{19f} are each individually selected from any amino acid;

X_{2f} is A, E, H, N, R, S, T, or V;

X_{4f} is A, G, N, S, or T;

X_{5f} is F, G, L, M, N, Q, S, T, or V;

X_{6f} is I, L, P, or V;

X_{9f} is I, L, T, or V;

X_{14f} is A, C, G, M, Q, R, S, or T;

X_{16f} is I, L, V, or Y;

X_{18f} is D, E, H, N, Q, or S;

X_{20f} is E, H, I, L, M, Q, R. or T;

X_{21f} is A, E, F, H, L, N, P, or Y;

X_{22f} is C, F, H, K, M, N, Q, R, T, or Y;

X_{23f} is D, E, F, I, K, L, N, Q, R, S, T, or V;

### Motif 7:

X_{1g}X_{2g}X_{3g}X_{4g}X_{5g}EX_{7g}X_{8g}X_{9g}X_{10g}X_{11g}X_{12g}RX_{14g}X_{15g}X_{16g}X_{17g}X_{18g}X_{19g}X_{20g}X_{21g}, wherein:

X_{2g}, X_{4g}, X_{8g}, X_{9g}, X_{11g}, X_{15g}, X_{17g}, and X_{20g} are each individually selected from any amino acid;

X_{1g} is A, G, I, N, S, T, or V;

X_{3g} is A, I, or S;

X_{5g} is F, I, L, M, or Y;

X_{7g} is I or R;

X_{10g} is D, I, L, or T;

X_{12g} is A, E, I, K, M, Q, or S;

X_{14g} is I, T, or V;

X_{16g} is A, D, G, R, S, or T;

X_{18g} is F, K, L, M, or Y;

X_{19g} is A, E, H, I, K, L, M, N, Q, R, V, W, or Y;

X_{21g} is A, I, K, L, M, or R

### Motif 8:

X₁ₕX₂ₕX₃ₕX₄ₕX₅ₕX₆ₕX₇ₕX₈ₕX₉ₕX₁₀ₕX₁₁ₕ, wherein:

X₆ₕ and X₁₀ₕ are each individually selected from any amino acid;

X₁ₕ is F or Y;

X₂ₕ is D, E, K, Q, or S;

X₃ₕ is E, K, L, M, or Q;

X₄ₕ is K, L, or R:

X₅ₕ is K, L, or V;

X₇ₕ is G or N;

X₈ₕ is D, E, H, K, L, M, or R;

X₉ₕ is S or T;

X₁₁ₕ is F, H, I, Q, S, T, V, or W

### Motif 9:

X₁ᵢX₂ᵢX₃ᵢX₄ᵢX₅ᵢX₆ᵢX₇ᵢX₈ᵢX₉ᵢX₁₀ᵢX₁₁ᵢX₁₃ᵢX₁₄ᵢX₁₅ᵢX₁₆ᵢX₁₇ᵢX₁₈ᵢX₁₉ᵢX₂₀ᵢX₂₁ᵢX₂₂ᵢX₂₃ᵢX₂₄ᵢX₂₅ᵢX₂₆ᵢX₂₇ᵢ, wherein:

X₂ᵢ, X₃ᵢ, X₅ᵢ, X₆ᵢ, X₇ᵢ, X₉ᵢ, X₁₃ᵢ, X₁₄ᵢ, X₁₇ᵢ, X₂₀ᵢ, X₂₄ᵢ, and X₂₆ᵢ are each individually selected from any amino acid;

X₁ᵢ is I, L, or V;

X₄ᵢ is A, D, F, H, I, L, M, N, Q, S, V, or Y;

X₈ᵢ is A, G, or S;

X₁₀ᵢ is D, E, I, K, N, Q, R, or S;

X₁₁ᵢ is E or Q;

X₁₅ᵢ is A or K;

X₁₆ᵢ is A, Q, R, or S;

X₁₈ᵢ is L, M, or R;

X₁₉ᵢ is I, L, Q, R, S, or V;

X₂₁ᵢ is A, D, E, G, H, I, Q. R, or S;

X₂₂ᵢ is A, K, N, Q, S, T, or V;

X₂₃ᵢ is A, H, K, R, W, or Y;

X₂₅ᵢ is A, G, H, I, K, Q, R, S, or T;

X₂₇ᵢ is C, H, I, K, L, R, or V

### Motif 10:

RX₂ⱼX₃ⱼX₄ⱼW, wherein:

X₂ⱼ is L, M, Q, or R;

X₃ⱼ is A, N, or S; and

X₄ⱼ is N, P, S, or T

### Motif 11:

X₁ₖX₂ₖX₃ₖX₄ₖX₅ₖX₆ₖX₇ₖX₈ₖF, wherein:

X₃ₖ and X₆ₖ are each individually selected from any amino acid;

X₁ₖ is I, L, or V;

X₂ₖ is A or V;

X₄ₖ is A, F, H, I, L, Q, W, or Y;

X₅ₖ is I, M, or V;

X₇ₖ is E, L, Q, or T:

X₈ₖ is A, I, or V

### Motif 12:

RX₂ₗX₃ₗX₄ₗX₅ₗX₆ₗX₇ₗX₈ₗX₉ₗX₁₀ₗX₁₁ₗX₁₂ₗX₁₃ₗ, wherein:

X₂ₗ is D, K, N, R, S, or V;

X₃ₗ is A, D, E, F, G, K, P, Q, or S;

X₄ₗ is A, E, I, K, L, S, T, or V;

X₅ₗ is any amino acid:

X₆ₗ is F, G, I, L, N, or V;

X₇ₗ is A, F, I, L, Q, R, V, or Y;

X₈ₗ is D, E, I, L, M, N, Q, S, T, or V;

X₉ₗ is D, E, F, I, L, M, Q, T, V, or Y;

X₁₀ₗ is I, K, L, R, or V;

X₁₁ₗ is D, E, K, N, Q, or R;

X₁₂ₗ is D, E, F, K, L, N, Q, W, or Y; and

X₁₃ₗ is F or L

### Motif 13:

X₁ₘX₂ₘX₃ₘX₄ₘX₅ₘX₆ₘX₇ₘX₈ₘX₉ₘX₁₀ₘX₁₁ₘX₁₂ₘX₁₃ₘX₁₄ₘX₁₅ₘX₁₆ₘX₁₇ₘX₁₈ₘX₁₉ₘX₂₀ₘX₂₁ₘX₂₂ₘX₂₃ₘX₂₄ₘ, wherein:

X₃ₘ, X₄ₘ, X₅ₘ, X₇ₘ, X₈ₘ, X₁₁ₘ, X₁₃ₘ, X₁₅ₘ, X₁₆ₘ, X₁₈ₘ, and X₂₂ₘ, are each individually selected from any amino acid,

X₁ₘ is A, E, F, I, L, M, N, Q, S, T, V, or Y;

X₂ₘ is A, F, G, I, L, M, R, S, T, or V;

X₆ₘ is A, D, E, F, G, H, L, M, N, S, or T;

X₉ₘ is D, M, N, or S;

X₁₀ₘ is D, E, or Q;

X₁₂ₘ is C, F, H, L, T, V, or Y;

X₁₄ₘ is A, E, K, L, R, or Y;

X₁₇ₘ is A, L, or S;

X₁₉ₘ is D, E, K, N, Q, R, or S;

X₂₀ₘ is G, I, M, Q, R, T, or V;

X₂₁ₘ is D, H, K, N, Q, or R;

X₂₃ₘ is A, G, I, L, N, S, T, or V;

X₂₄ₘ is F, H, I, K, L, M, N, Q, V, W, or Y

In some embodiments, the recombinase may comprise an amino acid sequence having at least 70% identity (e.g., at least 75%, at least 80%, at least 85%, at least 90%, at least 95%, at least 97%, at least 98%, at least 99%, or 100%) to any of amino acid motifs 1-13. The recombinase may also comprise enzymatically active fragments of the recited amino acid motifs (e.g., C- or N-terminal truncations or containing internal deletions, but retaining the desired enzymatic activity).

In some embodiments, the systems comprise a polypeptide comprising a recombinase having an amino acid sequence having at least 70% (e.g., at least 75%, at least 80%, at least 85%, at least 90%, at least 95%, at least 97%, at least 98%, at least 99%, or 100%) identity to any of SEQ ID NOs: 88-1183 (those listed in Tables 4 and 5). Also provided herein are enzymatically active fragments of SEQ ID NOs: 88-1183, from those sequences listed in Tables 4 and 5 (e.g., C- or N-terminal truncations or containing internal deletions, but retaining the desired enzymatic activity). The active fragment may contain at least 20 amino acids, at least 30 amino acids, at least 40 amino acids, at least 50 amino acids, at least 100 amino acids, or more of SEQ ID NOs: 88-1183 (Tables 4 and 5) or sequences at least 70% (e.g., at least 75%, at least 80%, at least 85%, at least 90%, at least 95%, at least 97%, at least 98%, at least 99%, or 100%) identity to at least 20 amino acids, at least 30 amino acids, at least 40 amino acids, at least 50 amino acids, at least 100 amino acids, or more of SEQ ID NOs: 88-1183 (Tables 4 and 5).

The term “recombinase,” as used herein, refers to a site-specific enzyme that mediates the recombination of DNA between recombinase recognition sequences, which results in the excision, integration, inversion, or exchange (e.g., translocation) of DNA fragments between the recombinase recognition sequences. In some embodiments, the recombinase is a large serine recombinase.

Large serine recombinases (LSRs) are site-specific recombinases that are commonly found on microbial mobile genetic elements and within phage genomes, allowing an invading phage to insert into the host genome and thus enter into their prophage state. The typical LSR is composed of distinct domains: an N-terminal “resolvase” domain that contains the active site; a “recombinase” domain that determines the DNA binding specificity of the enzyme; and a zinc beta ribbon domain and a coiled-coil motif implicated in additional binding specificity and irreversibility of forward integration reaction without excision cofactors. Based on detailed studies of the ΦC31 LSR, the following mechanism has been proposed: two LSR monomers bind to the donor attachment site and two bind to the acceptor attachment site—the four monomers come together to form a tetramer (FIG. 9). This complex then breaks both DNA strands and recombines them at the attachment sites to form a stably integrated final product.

The first polynucleotide may be a part of a bacterial plasmid, bacteriophage, plant virus, retrovirus. DNA virus, autonomously replicating extra chromosomal DNA element, linear plasmid, mitochondrial or other organellar DNA, chromosomal DNA, and the like. In some embodiments, the first polynucleotide comprises a human nucleic acid sequence. In some embodiments, the first polynucleotide is an exogenous or synthetic polynucleotide (e.g., a vector or engineered plasmid).

The first polynucleotide may comprise a donor recognition site for the recombinase. Recognition sites are specific polynucleotide sequences that are recognized by the recombinase enzymes described herein. The terms “attB” and “attP,” which refer to attachment (or recombination) sites originally from a bacterial target and a phage donor, respectively, are used herein although recombination sites for particular enzymes may have different names (e.g., “attD” and “attA”). The recombination sites typically include left and right arms separated by a core or spacer region.

In some embodiments, the first polynucleotide further comprises a cargo nucleic acid. The cargo nucleic acid may encode a gene product including but not limited to RNAs (e.g., non-coding RNA, such as tRNA, rRNA, micro RNA (miRNA), and small interfering RNA (siRNA), and coding RNA, such as messenger RNA (mRNA)) or proteins or polypeptides. The cargo nucleic acid may encode a transcription or translational control element (e.g., promoter elements, response elements (e.g., activator/repressor sequences)). In some embodiments, the cargo nucleic acid encodes a therapeutic protein. In some embodiments, the cargo nucleic acid encodes a therapeutic RNA.

The donor DNA, and by extension the cargo nucleic acid, may of any suitable length to facilitate recombination and delivery of the full cargo nucleic acid, including, for example, about 50-100 bp (base pairs), about 100-1000 bp, at least or about 10 bp, at least or about 20 bp, at least or about 25 bp, at least or about 30 bp, at least or about 35 bp, at least or about 40 bp, at least or about 45 bp, at least or about 50 bp, at least or about 55 bp, at least or about 60 bp, at least or about 65 bp, at least or about 70 bp, at least or about 75 bp, at least or about 80 bp, at least or about 85 bp, at least or about 90 bp, at least or about 95 bp, at least or about 100 bp, at least or about 200 bp, at least or about 300 bp, at least or about 400 bp, at least or about 500 bp, at least or about 600 bp, at least or about 700 bp, at least or about 800 bp, at least or about 900 bp, at least or about 1 kb (kilobase pair), at least or about 2 kb, at least or about 3 kb, at least or about 4 kb, at least or about 5 kb, at least or about 6 kb, at least or about 7 kb, at least or about 8 kb, at least or about 9 kb, at least or about 10 kb, or less than 10 kb, in length or greater. The donor DNA, and the cargo nucleic acid, may be at least or about 10 kb, at least or about 50 kb, at least or about 100 kb, between 20 kb and 60 kb, between 20 kb and 100 kb.

In essence, by contacting a set of corresponding recombination recognition sites with a corresponding recombinase, the recombinase mediates recombination between the sites. In some embodiments, the first polynucleotide further comprises a recipient recognition sequence for the recombinase.

In some embodiments, the system further comprises a second polynucleotide comprising a recipient recognition sequence for the recombinase. The second polynucleotide may be a part of a bacterial plasmid, bacteriophage, plant virus, retrovirus, DNA virus, autonomously replicating extra chromosomal DNA element, linear plasmid, mitochondrial or other organellar DNA, chromosomal DNA, and the like. In some embodiments, the second polynucleotide comprises a human nucleic acid sequence.

The type of recognition site will vary depending on the recombinase. In some embodiments, the recombinase is a landing-pad LSRs that can integrate efficiently at a pre-installed recognition site. Examples of landing-pad LSRs are shown in Table 1 along with their corresponding recombination attachment sites. In some embodiments, the recombinase is a multi-targeting LSRs that can integrate efficiently at many different loci in a target genome. Examples of a multi-targeting LSRs are shown in Table 3 along with their corresponding recombination attachment sites. In some embodiments, the recombinase is genome-targeting LSRs that can integrate at one or several target sites in a given target (e.g., target genome). Examples of genome-targeting LSRs are shown in Table 2 along with their corresponding recombination attachment sites. Attachment sites can be determined by mapping the edges of mobile genetic elements, as described herein.

In some embodiments, the donor recognition sequence, the recipient recognition sequence, or both are pseudo-recognition sequences or pseudosites. “Pseudo-recognition sequences” or “pseudosites” refer to a recognition sequences which is not necessarily that which is the native recognition sequence for a given recombinase but rather is sufficient to promote recombination. The pseudo-recognition sequence differs in one or more nucleotides from the corresponding native recombinase recognition sequence (e.g., due to insertions, deletions, or substitutions). In some embodiments, the pseudo-recognition sequence may be less than 50% identical to the native sequence. Pseudo-recognition sequences may also be those sequences present as an endogenous sequence in a genome that differs from the sequence of a genome where the wild-type recognition sequence for the recombinase resides. Identification of pseudo-recognition sequences can be accomplished, for example, by using sequence alignment and analysis, where the query sequence is the recognition sequence of interest, as described herein.

Depending upon the relative locations of the recombination attachment sites, any one of a number of events can occur as a result of the recombination. For example, if the recombination attachment sites are present on different nucleic acid molecules, the recombination can result in integration of one nucleic acid molecule into a second molecule.

The recombination attachment sites can also be present on the same nucleic acid molecule. In such cases, the resulting product typically depends upon the relative orientation of the attachment sites. For example, recombination between sites that are in the parallel or direct orientation will generally result in excision of any DNA that lies between the recombination attachment sites. In contrast, recombination between attachment sites that are in the reverse orientation can result in inversion of the intervening DNA.

The present disclosure also provides nucleic acids encoding the recombinases disclosed herein. The present disclosure further provides nucleic acids encoding the first polynucleotide and the second polynucleotide. The recombinase and the first polynucleotide may be encoded by the same or different nucleic acids (e.g., vectors). In some embodiments, a nucleic acid sequence encoding a recombinase is transiently or stable integrated into a cell, tissue, or organism so that the cell, tissue, or organism expresses the heterologous recombinase.

Nucleic acids of the present disclosure can comprise any of a number of promoters known to the art, wherein the promoter is constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g., enhancers, Kozak sequences and introns). Many promoter/regulatory sequences useful for driving constitutive expression of a gene are available in the art and include, but are not limited to, for example, CMV (cytomegalovirus promoter), EF1a (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), HI (human polymerase III RNA promoter), U6 (human U6 small nuclear promoter), and the like. Additional promoters that can be used for expression of the components of the present system, include, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters include any constitutively active promoter. Alternatively, any regulatable promoter may be used, such that its expression can be modulated within a cell.

Moreover, inducible expression can be accomplished by placing the nucleic acid encoding such a molecule under the control of an inducible promoter/regulatory sequence. Promoters that are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence known in the art that is capable of driving expression of the desired protein operably linked thereto.

The present disclosure also provides for vectors containing the nucleic acids or system and cells containing the nucleic acids or vectors, thereof. Thus, the disclosure further provides for cells comprising the serine recombinases or systems, as disclosed herein.

The vectors may be used to propagate the nucleic acid in an appropriate cell and/or to allow expression from the nucleic acid (e.g., an expression vector). The person of ordinary skill in the art would be aware of the various vectors available for propagation and expression of a nucleic acid sequence.

To construct cells that express the present system described herein, expression vectors for stable or transient expression of the present system may be constructed via conventional methods and introduced into cells. For example, nucleic acids may be cloned into a suitable expression vector, such as a plasmid or a viral vector in operable linkage to a suitable promoter. The selection of expression vectors/plasmids/viral vectors should be suitable for integration and replication in eukaryotic cells.

In certain embodiments, vectors of the present disclosure can drive the expression of one or more sequences in mammalian cells using a mammalian expression vector. Examples of mammalian expression vectors include pCDM8 (Seed, Nature (1987) 329:840, incorporated herein by reference) and pMT2PC (Kaufman, et al., EMBO J. (1987) 6:187, incorporated herein by reference). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd eds., Cold Spring Harbor Laboratory, Cold Spring Harbor Laboratory Press, Cold Spring Harbor, N.Y., 1989, incorporated herein by reference.

The vectors of the present disclosure may direct the expression of the nucleic acid in a particular cell type (e.g., tissue-specific regulatory elements are used to express the nucleic acid). Such regulatory elements include promoters that may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining.

Additionally, the vector may contain, for example, some or all of the following: a selectable marker gene for selection of stable or transient transfectants in host cells; transcription termination and RNA processing signals; 5′- and 3-untranslated regions; internal ribosome binding sites (IRESes), versatile multiple cloning sites; and reporter gene for assessing expression of the chimeric receptor. Suitable vectors and methods for producing vectors containing transgenes are well known and available in the art. Selectable markers include chloramphenicol resistance, tetracycline resistance, spectinomycin resistance, neomycin, streptomycin resistance, erythromycin resistance, rifampicin resistance, bleomycin resistance, thermally adapted kanamycin resistance, gentamycin resistance, hygromycin resistance, trimethoprim resistance, dihydrofolate reductase (DHFR), GPT; the URA3, HIS4, LEU2, and TRP1 genes of S. cerevisiae.

Conventional viral and non-viral based gene transfer methods can be used to introduce the nucleic acids into cells, tissues, or a subject. Such methods can be used to administer the nucleic acids to cells in culture, or in a host organism. Non-viral vector delivery systems include DNA plasmids, cosmids, RNA (e.g., a transcript of a vector described herein), a nucleic acid, and a nucleic acid complexed with a delivery vehicle.

The nucleic acids may be delivered by any suitable means. In certain embodiments, the nucleic acids or proteins thereof are delivered in vivo. In other embodiments, the nucleic acids or proteins thereof are delivered to isolated/cultured cells in vitro or ex vivo to provide modified cells useful for in vivo delivery to patients afflicted with a disease or condition.

Vectors according to the present disclosure can be transformed, transfected, or otherwise introduced into a wide variety of host cells. Transfection refers to the taking up of a vector by a cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

Methods of delivering vectors to cells are well known in the art and may include DNA or RNA electroporation, transfection reagents such as liposomes or nanoparticles to delivery DNA or RNA; delivery of DNA, RNA, or protein by mechanical deformation (see, e.g., Sharei et al. Proc. Natl. Acad. Sci. USA (2013) 110(6): 2082-2087, incorporated herein by reference. Nucleic acids can be delivered as part of a larger construct, such as a plasmid or viral vector, or directly, e.g., by electroporation, lipid vesicles, viral transporters, microinjection, and biolistics (high-speed particle bombardment).

Additionally, delivery vehicles such as nanoparticle- and lipid-based delivery systems can be used. Further examples of delivery vehicles include lentiviral vectors, ribonucleoprotein (RNP) complexes, lipid-based delivery system, gene gun, hydrodynamic, electroporation or nucleofection microinjection, and biolistics. Various gene delivery methods are discussed in detail by Nayerossadat et al. (Adv Biomed Res. 2012: 1: 27) and Ibraheem et al. (Int J Pharm. 2014 Jan. 1; 459(1-2):70-83), incorporated herein by reference.

As such, the disclosure provides an isolated cell comprising the vector(s) or nucleic acid(s) disclosed herein. Preferred cells are those that can be easily and reliably grown, have reasonably fast growth rates, have well characterized expression systems, and can be transformed or transfected easily and efficiently. Examples of suitable prokaryotic cells include, but are not limited to, cells from the genera Bacillus (such as Bacillus subtilis and Bacillus brevis), Escherichia (such as E. coli), Pseudomonas, Streptomyces, Salmonella, and Envinia. Suitable eukaryotic cells are known in the art and include, for example, yeast cells, insect cells, and mammalian cells. Examples of suitable yeast cells include those from the genera Kluyveromyces, Pichia, Rhino-sporidium, Saccharomyces, and Schizosaccharomyces. Exemplary insect cells include Sf-9 and HIS (Invitrogen, Carlsbad, Calif.) and are described in, for example, Kitts et al., Biotechniques, 14: 810-817 (1993); Lucklow, Curr. Opin. Biotechnol., 4: 564-572 (1993); and Lucklow et al., J. Virol., 67: 4566-4579 (1993), incorporated herein by reference. Desirably, the cell is a mammalian cell, and in some embodiments, the cell is a human cell. A number of suitable mammalian and human host cells are known in the art, and many are available from the American Type Culture Collection (ATCC, Manassas, Va.). Examples of suitable mammalian cells include, but are not limited to, Chinese hamster ovary cells (CHO) (ATCC No. CCL61), CHO DHFR-cells (Urlaub et al., Proc. Natl. Acad. Sci. USA, 97: 4216-4220 (1980)), human embryonic kidney (HEK) 293 or 293T cells (ATCC No. CRL1573), and 3T3 cells (ATCC No. CCL92). Other suitable mammalian cell lines are the monkey COS-1 (ATCC No. CRL1650) and COS-7 cell lines (ATCC No. CRL1651), as well as the CV-1 cell line (ATCC No. CCL70). Further exemplary mammalian host cells include primate, rodent, and human cell lines, including transformed cell lines. Normal diploid cells, cell strains derived from in vitro culture of primary tissue, as well as primary explants, are also suitable. Other suitable mammalian cell lines include, but are not limited to, mouse neuroblastoma N2A cells, HeLa, HEK, A549, HepG2, mouse L-929 cells, and BHK or HaK hamster cell lines.

Methods for selecting suitable mammalian cells and methods for transformation, culture, amplification, screening, and purification of cells are known in the art.

The present invention is also directed to compositions comprising a recombinase, a system, a nucleic acid, a vector, or a cell, as described herein.

Further disclosed herein are methods for identifying recombinases for use in the systems and methods disclosed herein. In some embodiments, the methods comprise: acquiring bacterial genome sequences; identifying putative recombinase genes in the bacterial genome sequences based on predicted recombinase domain; comparing genomes encoding the putative recombinase genes with those without the putative recombinase genes; mapping boundaries of a mobile genetic element comprising the putative recombinase genes: determine recombinase recognition sequences and/or attachment sites. In some embodiments, the predicted recombinase domain is a Pfam domain. In some embodiments, the method further comprises isolating mobile genetic elements from the bacterial genome sequences prior to identifying the putative recombinase genes. Mapping boundaries of a mobile genetic element may comprise determining 3′ and 5′ flanking sequences of the mobile genetic element termini and, if present, the duplication sites created upon insertion of the mobile genetic element.

### 3. METHODS OF ALTERING DNA

Applications of genetic engineering through alteration of DNA has yielded impactful results including CAR-T cell therapies, genetically modified crops, and cells producing diverse compounds and medicines. In many of these applications, genomic integration is highly preferred over plasmid-based methods for maintaining heterologous genes in engineered cells, due to improved stability in the genome, better control of copy numbers, and regulatory concerns regarding biocontainment of recombinant DNA. However, generation of modified cells with kilobases of changes across the genome remains practically challenging, often requiring inefficient, multi-step processes that are time and resource intensive. The systems and methods described herein allow integration of a large (e.g., kilobase or larger) exogenous donor polynucleotide into a DNA sequence. The methods may be used in vitro, ex vivo, or in vivo and allow alteration of a target DNA strand in solution, in a cell, in a tissue, or in a subject.

The disclosure provides a method of altering a target nucleic acid sequence. The phrases “altering a DNA sequence” or “altering a target DNA,” as used herein, refer to modifying at least one physical feature of a DNA sequence of interest. DNA alterations include, for example, single or double strand DNA breaks, deletion, or insertion of one or more nucleotides, and other modifications that affect the structural integrity or nucleotide sequence of the DNA sequence.

In some embodiments, the methods comprise contacting a target nucleic acid sequence with a system disclosed herein or with a polypeptide comprising a recombinase having an amino acid sequence having at least 70% (e.g., at least 75%, at least 80%, at least 85%, at least 90%, at least 95%, at least 97%, at least 98%, at least 99%, or 100%) identity to any of SEQ ID NOs: 1-74, an enzymatically active fragment thereof, or a nucleic acid encoding thereof.

In some embodiments, the recombinase has an amino acid sequence having at least 70% (e.g., at least 75%, at least 80%, at least 85%, at least 90%, at least 95%, at least 97%, at least 98%, at least 99%, or 100%) identity to any of SEQ ID NOs: 2, 6, 10, 12, 18, 19, 26, 29, 61, 65, or 66. In select embodiments, the recombinase has an amino acid sequence of SEQ ID NOs: 2, 6, 10, 12, 18, 19, 26, 29, 61, 65, or 66.

In some embodiments, the methods comprise contacting a target nucleic acid sequence with a system disclosed herein or with a polypeptide comprising a recombinase having an amino acid sequence having at least 70% (e.g., at least 75%, at least 80%, at least 85%, at least 90%, at least 95%, at least 97%, at least 98%, at least 99%, or 100%) identity to any of motifs 1-13 as disclosed above, an enzymatically active fragment thereof, or a nucleic acid encoding thereof.

In some embodiments, the systems comprise a polypeptide comprising a recombinase having an amino acid sequence having at least 70% (e.g., at least 75%, at least 80%, at least 85%, at least 90%, at least 95%, at least 97%, at least 98%, at least 99%, or 100%) identity to any of SEQ ID NOs: 88-1183, those listed in Tables 4 and 5. Also provided herein are enzymatically active fragments of SEQ ID NOs: 88-1183, those sequences listed in Tables 4 and 5 (e.g., C- or N-terminal truncations or containing internal deletions, but retaining the desired enzymatic activity). The active fragment may contain at least 20 amino acids, at least 30 amino acids, at least 40 amino acids, at least 50 amino acids, at least 100 amino acids, or more of SEQ ID NOs: 88-1183 (Tables 4 and 5) or sequences at least 70% (e.g., at least 75%, at least 80%, at least 85%, at least 90%, at least 95%, at least 97%, at least 98%, at least 99%, or 100%) identity to at least 20 amino acids, at least 30 amino acids, at least 40 amino acids, at least 50 amino acids, at least 100 amino acids, or more of SEQ ID NOs: 88-1183 (Tables 4 and 5).

In some embodiments, the target DNA comprises a donor recognition sequence, a recipient recognition sequence, or both.

In some embodiments, the methods further comprise contacting the target DNA with a first polynucleotide comprising a donor recognition sequence for the recombinase. In some embodiments, the first polynucleotide further comprises a cargo DNA sequence. In some embodiments, the donor recognition sequence, the recipient recognition sequence, or both are pseudo-recognition sequences.

The descriptions and embodiments provided above for the disclosed system, recombinase, first and second polynucleotide, donor and recipient recognition sequences, and cargo DNA sequence are applicable to the methods described herein.

In some embodiments, the methods may comprise introducing the disclosed systems or recombinase, or a nucleic acid encoding thereof, and a donor polynucleotide into a cell. In some embodiments, the recombinase, or the nucleic acid encoding thereof, is introduced into the cell before the introduction of the donor polynucleotide. In some embodiments, the recombinase, or the nucleic acid encoding thereof, is introduced into the cell after the introduction of the donor polynucleotide. In some embodiments, the recombinase, or the nucleic acid encoding thereof, and the donor polynucleotide may be introduced, in any order, with a time period separating each introduction.

In some embodiments, the recombinase is part of a system comprising a Cas protein, a reverse transcriptase, or active fragments or combinations thereof. In some embodiments, the recombinase is in a fusion protein with a Cas protein (e.g., Cas 9) and a reverse transcriptase, or active fragments thereof. For example, a Programmable Addition via Site-specific Targeting Elements (PASTE) system which integrates large cargos in a single delivery. See, Eleonora I. Ioannidi, et al., bioRxiv 2021.11.01.466786, incorporated herein by reference in its entirety.

In some embodiments, the recombinase, or the nucleic acid encoding thereof, is introduced into the cell concurrently with the introduction of the donor polynucleotide. For example, the recombinase, or the nucleic acid encoding thereof, and the donor polynucleotide are introduced simultaneously or nearly simultaneously.

The cell can be a mitotic and/or post-mitotic cell from any eukaryotic cell or organism (e.g. a cell of a single-cell eukaryotic organism, a plant cell, an algal cell, a fungal cell (e.g., a yeast cell), an animal cell, a cell from an invertebrate animal (e.g. fruit fly, cnidarian, echinoderm, nematode, an insect, an arachnid, etc.), a cell from a vertebrate animal (e.g., fish, amphibian, reptile, bird, mammal), a cell from a mammal, a cell from a rodent, a cell from a human, etc.), or a protozoan cell. Any type of cell may be of interest (e.g. a stem cell, e.g. an embryonic stem (ES) cell, an induced pluripotent stem (iPS) cell, a germ cell; a somatic cell, e.g. a fibroblast, a hematopoietic cell, a neuron, a muscle cell, a bone cell, a hepatocyte, a pancreatic cell, a liver cell, a lung cell, a skin cell; an in vitro or in vivo embryonic cell of an embryo at any stage, e.g., a 1-cell, 2-cell, 4-cell, 8-cell, etc. stage zebrafish embryo; etc.). Cells may be from established cell lines or they may be primary cells, where “primary cells,” “primary cell lines,” and “primary cultures” are used interchangeably herein to refer to cells and cells cultures that have been derived from a subject and allowed to grow in vitro for a limited number of passages.

In some embodiments, the one or more cells are animal cells. The present disclosure provides for a modified animal cell produced by the present system and method, an animal comprising the animal cell, a population of cells comprising the cell, tissues, and at least one organ of the animal. The present disclosure further encompasses the progeny, clones, cell lines or cells of the genetically modified animal. The present cells may be used for transplantation (e.g., hematopoietic stem cells or bone marrow).

Non-limiting examples of animal cells that may be genetically modified using the systems and methods include, but are not limited to, cells from: mammals such as primates (e.g., ape, chimpanzee, macaque), rodents (e.g., mouse, rabbit, rat), canine or dog, livestock (cow/bovine, donkey, sheep/ovine, goat or pig), fowl or poultry (e.g., chicken), and fish (e.g., zebra fish). The present methods and systems may be used for cells from other eukaryotic model organisms, e.g., Drosophila, C. elegans, etc. In certain embodiments, the mammal is a human, a non-human primate (e.g., marmoset, rhesus monkey, chimpanzee), a rodent (e.g., mouse, rat, gerbil, Guinea pig, hamster, cotton rat, naked mole rat), a rabbit, a livestock animal (e.g., goat, sheep, pig, cow, cattle, buffalo, horse, camelid), a pet mammal (e.g., dog, cat), a zoo mammal, a marsupial, an endangered mammal, and an outbred or a random bred population thereof.

In some embodiments, the one or more cells comprise plant cells. Suitable plant cells may be from a number of different plants including, but are not limited to, monocotyledonous and dicotyledonous plants, such as crops including grain crops (e.g., wheat, maize, rice, millet, barley), fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rapeseed) and plants used for experimental purposes (e.g., Arabidopsis). Thus, the disclosed methods and compositions have use over a broad range of plants, including, but not limited to, species from the genera Asparagus, Avena, Brassica, Citrus, Citrullus, Capsicum, Cucurbita, Daucus, Glycine, Hordeum, Lactuca, Lycopersicon, Malus, Manihot, Nicotiana, Oryza, Persea, Pisum, Pyrus, Prunus, Raphanus, Secale, Solanum, Sorghum, Triticum, Vitis, Vigna, and Zea.

In some embodiments, the one or more cells comprise microbial cells. In some embodiments, the microbial cells are Gram-negative bacterial cells, Gram-positive bacterial cells, or a combination thereof. In some embodiments, the microbial cells are pathogenic bacterial cells. In some embodiments, the microbial cells are non-pathogenic bacterial cells (e.g., probiotic and/or commensal bacterial cells). In some embodiments, the microbial cells form microbial flora (e.g., natural human microbial flora). In some embodiments, the microbial cells are used in industrial or environmental bioprocesses (e.g., bioremediation).

The cell can be a cancer cell. An appropriate cancer cell can be derived from a breast cancer, lung cancer, colon cancer, pancreatic cancer, renal cancer, stomach cancer, liver cancer, bone cancer, hematological cancer (e.g., leukemia or lymphoma), neural tissue cancer, melanoma, ovarian cancer, testicular cancer, prostate cancer, cervical cancer, vaginal cancer, or bladder cancer.

The systems and methods may be used to modify a stem cell. The term “stem cell” is used herein to refer to a cell that has the ability both to self-renew and to generate a differentiated cell type (see Morrison et al. (1997) Cell 88:287-298, incorporated herein by reference). Stem cells may be characterized by both the presence of specific markers (e.g., proteins, RNAs, etc.) and the absence of specific markers. Stem cells may also be identified by functional assays both in vitro and in vivo, particularly assays relating to the ability of stem cells to give rise to multiple differentiated progeny. Examples of stem cells include pluripotent, multipotent and unipotent stem cells. Examples of pluripotent stem cells include embryonic stem cells, embryonic germ cells, embryonic carcinoma cells and induced pluripotent stem cells (iPSCs). The cell may be an induced pluripotent stem cell (iPSC), e.g., derived from a fibroblast of a subject. In another embodiment, the cell can be a fibroblast. In some embodiments, the cell may be a cancer stem cell.

The present disclosure further provides progeny of a genetically modified cell, where the progeny can comprise the same genetic modification as the genetically modified cell from which it was derived. The present disclosure further provides a composition comprising a genetically modified cell. In some embodiments, a genetically modified host cell can generate a genetically modified organism. For example, the genetically modified host cell is a pluripotent stem cell, it can generate a genetically modified organism. Methods of producing genetically modified organisms are known in the art.

In some embodiments, the cell is in an organism or host, such that introducing the disclosed recombinases, systems, compositions, nucleic acids, or vectors into the cell comprises administration to a subject. The method may comprise providing or administering to the subject, in vivo, or by transplantation of ex vivo treated cells, a recombinase, nucleic acid, vector, composition, or system as described herein.

Cell replacement therapy can be used to prevent, correct, or treat a disease or condition, where the methods of the present disclosure are applied to isolated subject's cells (ex vivo), which is then followed by the administration of the genetically modified cells into the patient.

The cell may be autologous or allogeneic to the subject who is administered the cell. As described herein, the genetically modified cells may be autologous to the subject, e.g., the cells are obtained from the subject in need of the treatment, genetically engineered, and then administered to the same subject. Alternatively, the host cells are allogeneic cells, e.g., the cells are obtained from a first subject, genetically engineered, and administered to a second subject that is different from the first subject but of the same species. In some embodiments, the genetically modified cells are allogeneic cells and have been further genetically engineered to reduced graft-versus-host disease.

A “subject” may be human or non-human and may include, for example, animal strains or species used as “model systems” for research purposes, such a mouse model as described herein. Likewise, subject may include either adults or juveniles (e.g., children). Moreover, subject may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods and compositions provided herein, the mammal is a human.

The methods find use in inactivating a gene of interest or deleting a nucleic acid sequence. In some embodiments, the disclosed methods alter a target genomic DNA sequence in a host cell, tissue, or subject so as to modulate expression of the target DNA sequence, e.g., expression of the target DNA sequence is increased, decreased, or completely eliminated (e.g., via deletion of a gene or insertion or inversion of a promoter element). In some embodiments, the systems and methods described herein may be used to introduce an exogenous donor polynucleotide into a target DNA sequence.

In some embodiments, the target DNA encodes a gene product. The term “gene product,” as used herein, refers to any biochemical product resulting from expression of a gene. Gene products may be RNA or protein. RNA gene products include non-coding RNA, such as tRNA, rRNA, micro RNA (miRNA), and small interfering RNA (siRNA), and coding RNA, such as messenger RNA (mRNA). In some embodiments, the target genomic DNA sequence encodes a protein or polypeptide. However, the invention is not limited to editing of gene products. Any target DNA sequence may be edited, as desired. For example, in some embodiments, target DNA comprises non-coding DNA or comprises regions which are responsible for producing RNA. In some embodiments, the gene of interest is located chromosomally. In some embodiments, the gene of interest is located episomally, e.g., in bacterial cells.

Methods for inactivating a gene of interest comprise introducing into one or more cells the recombinases, systems, nucleic acids, or vectors described herein, wherein the target nucleic acid sequence comprises at least a portion of the gene of interest. The gene of interest may comprise any gene of interest to inactivate. In some embodiments, the gene of interest comprises an antibiotic resistance gene, a virulence gene, a metabolic gene, a toxin gene, a remodeling gene, a gene or gene variant responsible for a disease, or a mutant gene.

In select embodiments, the systems and methods described herein may be used to correct one or more defects or mutations in a gene (referred to as “gene correction”). In such cases, the cell or target sequence encodes a defective version of a gene, and the disclosed system further comprises a cargo nucleic acid molecule which encodes a wild-type or corrected version of the gene. Thus, in other words, the cell expresses a “disease-associated” gene. The term “disease-associated gene,” refers to any gene or polynucleotide whose gene products are expressed at an abnormal level or in an abnormal form in cells obtained from a disease-affected individual as compared with tissues or cells obtained from an individual not affected by the disease. A disease-associated gene may be expressed at an abnormally high level or at an abnormally low level, where the altered expression correlates with the occurrence and/or progression of the disease. A disease-associated gene also refers to a gene, the mutation or genetic variation of which is directly responsible or is in linkage disequilibrium with a gene(s) that is responsible for the etiology of a disease. Examples of genes responsible for such “single gene” or “monogenic” diseases include, but are not limited to, adenosine deaminase, α-1 antitrypsin, cystic fibrosis transmembrane conductance regulator (CFTR), β-hemoglobin (HBB), oculocutaneous albinism II (OCA2), Huntingtin (HTT), dystrophia myotonica-protein kinase (DMPK), low-density lipoprotein receptor (LDLR), apolipoprotein B (APOB), neurofibromin 1 (NF1), polycystic kidney disease 1 (PKD1), polycystic kidney disease 2 (PKD2), coagulation factor VIII (F8), dystrophin (DMD), phosphate-regulating endopeptidase homologue, X-linked (PHEX), methyl-CpG-binding protein 2 (MECP2), and ubiquitin-specific peptidase 9Y, Y-linked (USP9Y). Other single gene or monogenic diseases are known in the art and described in, e.g., Chial, H. Rare Genetic Disorders: Learning About Genetic Disease Through Gene Mapping, SNPs, and Microarray Data, Nature Education 1(1):192 (2008); Online Mendelian Inheritance in Man (OMIM); and the Human Gene Mutation Database (HGMD). In another embodiment, the target genomic DNA sequence can comprise a gene, the mutation of which contributes to a particular disease in combination with mutations in other genes. Diseases caused by the contribution of multiple genes which lack simple (i.e., Mendelian) inheritance patterns are referred to in the art as a “multifactorial” or “polygenic” disease. Examples of multifactorial or polygenic diseases include, but are not limited to, asthma, diabetes, epilepsy, hypertension, bipolar disorder, and schizophrenia. Certain developmental abnormalities also can be inherited in a multifactorial or polygenic pattern and include, for example, cleft lip/palate, congenital heart defects, and neural tube defects.

### 4. KITS

Also within the scope of the present disclosure are kits including a recombinase, or nucleic acid encoding thereof, a donor or first polynucleotide, a composition, or system as described herein, or a cell comprising a system as described herein or a recombinase as described herein.

The kits can also comprise instructions for using the components of the kit. The instructions are relevant materials or methodologies pertaining to the kit. The materials may include any combination of the following: background information, list of components, brief or detailed protocols for using the compositions, trouble-shooting, references, technical support, and any other related documents. Instructions can be supplied with the kit or as a separate member component, either as a paper form or an electronic form which may be supplied on computer readable memory device or downloaded from an internet website, or as recorded presentation.

It is understood that the disclosed kits can be employed in connection with the disclosed methods. The kit may include instructions for use in any of the methods described herein. The instructions can comprise a description of use of the components for the methods of identifying recombinases or methods of altering DNA.

The kits provided herein are in suitable packaging. Suitable packaging includes, but is not limited to, vials, bottles, jars, flexible packaging, and the like.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

The kit may further comprise a device for holding or administering the present recombinase, nucleic acids, system, or composition. The device may include an infusion device, an intravenous solution bag, a hypodermic needle, a vial, and/or a syringe.

The present disclosure also provides for kits for performing the methods or producing the components in vitro. The kit may include the components of the present system. Optional components of the kit include one or more of the following: (1) buffer constituents. (2) control plasmid, (3) transfection or transduction reagents.

### 5. EXAMPLES

Cell lines and cell culture. K562 (ATCC CCL-243) cells were cultured in a controlled humidified incubator at 37° C. and 5% CO₂, in RPMI 1640 (Gibco) media supplemented with 10% FBS (Hyclone), penicillin (10,000 I.U./mL), streptomycin (10,000 μg/mL), and L-glutamine (2 mM). HEK-293T cells, as well as HEK-293FT and HEK-293T-LentiX cells used to produce lentivirus, as described below, were grown in DMEM (Gibco) media supplemented with 10% FBS (Hyclone), penicillin (10,000 I.U./mL), and streptomycin (10,000 μg/mL).

Selecting large serine recombinases (LSRs) for initial pilot experiments. LSRs for the pilot experiments were identified by searching for the Recombinase Pfam domain among the mobile genetic elements (MGEs) previously identified (See Durrant et al. (2020) Cell Host & Microbe 28(5): 767 and El-Gebali et al., Nucleic Acids Res. 47, D427-D432 (2019), incorporated herein by reference in their entirety). The identity of the attachment site was inferred from the boundaries of the MGE that contained each LSR. For example, if a sequence had the following structure:

B₁-D-P₁-E-P₂-D-B₂

where B₁ indicates the sequence flanking the MGE insertion on the 5′ end, D indicates the target site duplication created upon insertion (if it exists), P₁ indicates the sequence flanking the 5′ integration boundary that is included in the MGE, E is the intervening MGE, P₂ indicates the sequence flanking the 3′ integration boundary that is included in the MGE, and B2 indicates the sequence flanking the MGE insertion on the 3′ end, then the attB and attP sequences can be reconstructed as:

attB=B₁+D+B₂

attP=P₂+D+P₁

where the “+” operator in this case indicates nucleotide sequence concatenation.

Candidates were then annotated to determine features such as: 1) whether or not the element was predicted to be a phage element, 2) how many isolates contain the integrated MGE, and 3) how often MGEs containing distinct LSRs will integrate at the same location in the genome. Candidates were then given higher priority if they were contained within predicted phage elements, if they appeared in multiple isolates, and if the attachment sites were targeted by multiple distinct LSRs.

Computational workflow to identify thousands of LSRs and cognate attachment sites. The LSR-identification workflow was implemented as described schematically in FIG. 9. 146,028 bacterial isolate genomes available in the NCBI RefSeq database were identified. Genomes were then clustered at the species level using the NCBI taxon ID and the TaxonKit tool. Genomes within each species were randomized and batched into sets of 50 and 20 genomes, where the first batch included 50 genomes and all subsequent batches contained 20 genomes. Each batch was then processed by downloading all relevant genomes from NCBI, annotating coding sequences in each genome with Prodigal, and then searching for all encoded proteins that contained a predicted Recombinase Pfam domain using HMMER (El-Gebali et al., 2019; HMMER, n.d.). Genomes that contained a predicted LSR were then compared to genomes that lacked that same LSR using the MGEfinder command wholegenome, which was developed by adapting the default MGEfinder to work with draft genomes. If MGE boundaries that contained the LSR were identified, all of the relevant sequence data was saved and stored in a database. The workflow was parallelized using Google Cloud virtual machines.

After this initial round of LSR mining was complete, a modified approach was taken to further expand the database and avoid redundant searches. First, bacterial species with a high number of isolate genomes available in the first round of LSR mining were analyzed to determine if further mining of these genomes would be necessary. Rarefaction curves representing the number of new LSR families identified with each additional genome analyzed were estimated for these common species, and species that appeared saturated (e.g., less than 1 new cluster per 1000 genomes analyzed) were considered “complete,” meaning no further genomes belonging to this species would be analyzed. Next, 48,557 genomes that met these filtering criteria were downloaded from the GenBank database and prepared for further analysis. The analysis was very similar to round 1, but with some notable differences. First, a database of over 496,133 isolate genomes from the RefSeq and GenBank genomes was constructed. PhyloPhlAn marker genes were then extracted from all of these genomes. Next, for each genome that was found to contain a given LSR, closely related isolates found in the database were selected according to marker gene homology were then selected for the comparative genomics analysis and further LSR discovery. This marker gene search approach was made available in a public github repository (github(dot)com(backslash)bhattlab(backslash)GenomeSearch). This second round of LSR and attachment site mining increased the total number of candidates by approximately 32%.

Predicting LSR target site specificity. LSR protein sequences were clustered at 90% and 50% identity using MMseqs2. Protein sequences that overlapped with predicted attachment sites were extracted from their genome of origin and clustered with all other target proteins at 50% identity using MMseqs2. LSR-attachment site combinations that were found to meet intermediate quality control filters were considered. To identify site-specific LSRs, only LSRs clustered at 50% identity and target proteins clustered at 50% identity were considered. Next, LSR-target pairs were filtered to only include target protein clusters that were targeted by 3 or more LSR clusters. Next, only LSR clusters that targeted a single target protein cluster were considered. The remaining sets of LSR clusters were considered to be single-targeting, meaning that they likely site-specifically targeted only one protein cluster. Multi-targeting, or transposable LSRs with minimal site-specificity, were identified. Only LSRs clustered at 90% identity and target proteins clustered at 50% identity were considered. Next, the total number of target protein clusters that were targeted by each LSR cluster were counted, and LSR clusters that targeted only one protein cluster were removed from consideration. Next, the remaining LSRs were binned according to the number of protein clusters that they targeted, where “2” indicates two target proteins, “3” indicates three target proteins, and “>3” indicates more than three target proteins. As referred to herein, “2” and “3” are considered moderately multi-targeting, while “>3” are considered fully multi-targeting. Each 50% identity cluster was then assigned to a multi-targeting bin according to the highest bin attained by any one 90% cluster found within the 50% identity cluster.

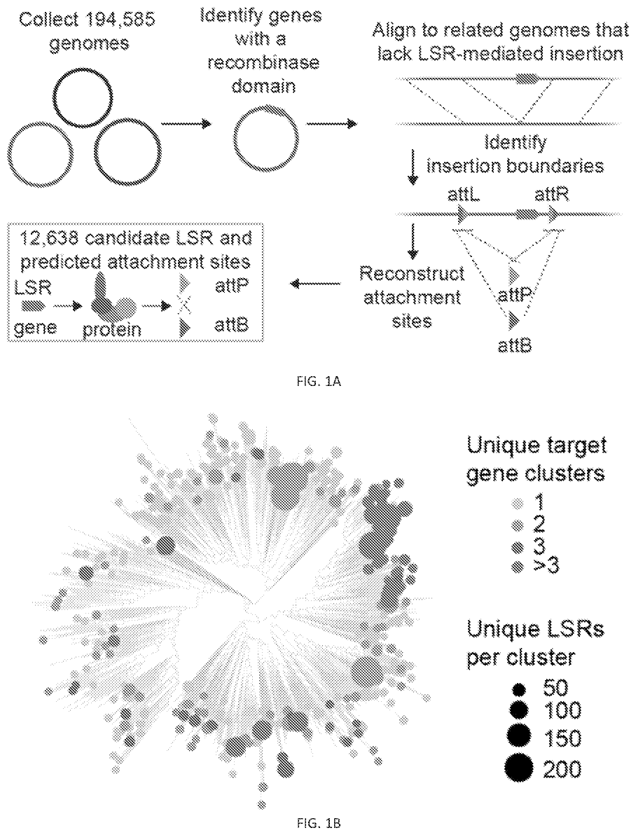

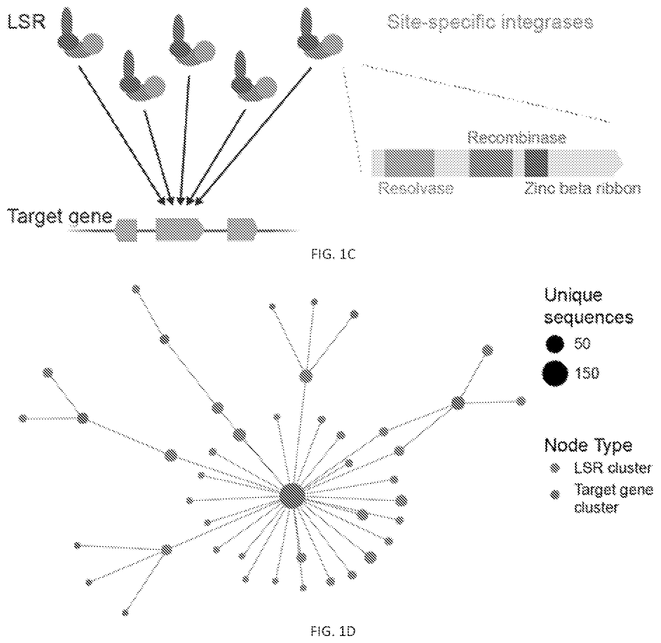

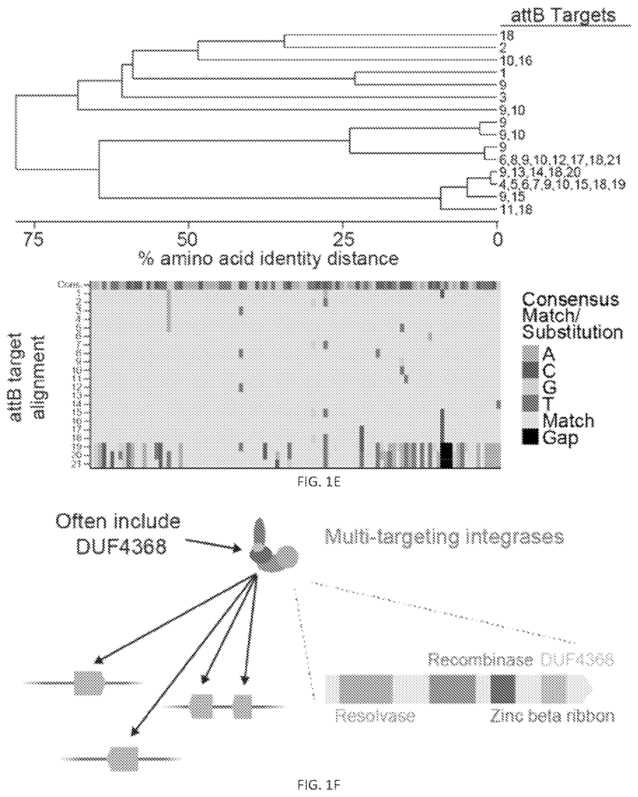

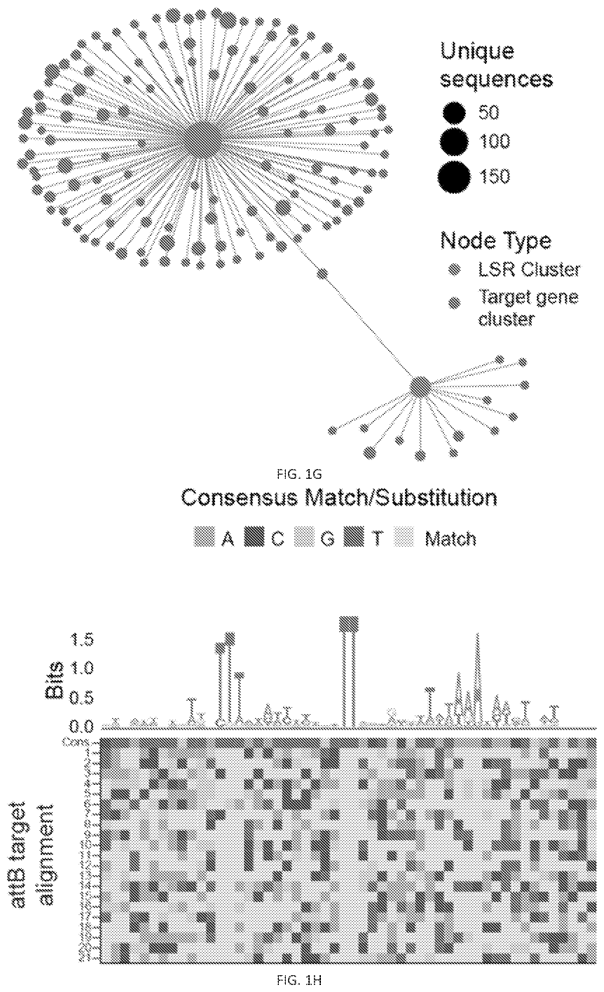

Phylogenetic analysis of site-specific integrases targeting a conserved attachment site. One example of several site-specific integrases targeting a conserved attachment site is shown in FIG. 1E. All attB attachment sites were clustered at 80% identity using MMseqs2. Candidates were filtered to include only those that met QC thresholds, and then attB sites that were ranked by the number of LSR clusters that were found to target them. An example attB cluster was chosen for further analysis. All LSRs that targeted this attB cluster were extracted from the database, and were aligned using the MAFFT-LINSI algorithm. Amino acid identity distances between all LSRs were calculated, and the distance matrix was used to create a hierarchical tree in R. LSRs that were 99% identical at the amino acid level or more were collapsed into a single cluster. This hierarchical tree was visualized and shown in FIG. 1E, along with all attB sites that were targeted by the LSRs.

Identifying target site motifs from attachment sites in the LSR database. Multi-targeting LSRs in the database were analyzed at the level of individual proteins, at the level of 90% amino acid identity clusters, and at the level of 50% amino acid identity clusters. For each of these levels, only candidates that were found to target more than 10 unique attB sequences or 10 target genes clustered at 50% amino acid identity were kept. Then all of the corresponding attB sequences were extracted, with only one attachment site per target gene cluster being extracted to avoid redundancy. These attB sequences were then initially aligned using MAFFT-LINSI. Next, possible core dinucleotides were identified in each alignment by extracting all dinucleotides in the alignment, and ranking them by the conservation of their most frequent nucleotides and their proximity to the center of the attB sequences, using a custom score that equally weighted high nucleotide conservation and normalized distance to the attB center. Candidates were then re-aligned only with respect to these predicted dinucleotide cores, rather than using an alignment algorithm such as MAFFT. These alignments were then visualized in using ggseqlogo to identify conserved target site motifs.

Quality controls and selection criteria for LSRs. LSRs with large attachment site cores, above 20 base pairs in length, were removed. The attachment site core is the portion of the attB and the attP that are predicted to be perfectly homologous. LSRs with attachment sites with more than 5% of their nucleotides being ambiguous in the original genome assemblies were removed. Only LSRs between 400 amino acids and 650 amino acids were kept. Next, only predicted LSRs that contained at least one of the three main LSR Pfam domains were retained (Resolvase. Recombinase, and Zn_ribbon_recom). Next, LSRs were removed from consideration if their sequences contained more than 5% ambiguous amino acids. Only LSRs that were found on integrative mobile genetic elements that were less than 200 kilobases in length were retained. And finally, only LSRs that were within 500 nucleotides of their predicted attachment sites were retained. Candidates that met all of these filters were considered to meet quality-control thresholds.

Plasmid recombination assay to validate LSR-attD-attA predictions. Three plasmids were designed for each LSR candidate. The effector plasmid contained the EF1a promoter, followed by the recombinase coding sequence (codon optimized for human cells), a 2A self-cleaving peptide, and an eGFP coding sequence. The attA plasmid contained an EF1a promoter, followed by the attA sequence, followed by mTagBFP2 coding sequence, which should constitutively express the mTagBFP2 protein in human cells. The attD plasmid included only the attD sequence followed by the mCherry coding sequence, which should produce no fluorescent mCherry prior to integration. HEK-293T cells were plated into 96 well plates and transfected one day later with 200 ng of effector plasmid, 70 ng of attA plasmid, and 50 ng of attD plasmid using Lipofectamine 2000 (Invitrogen). 2-3 days after transfection of cells with all three plasmids, cells were then measured using flow cytometry on an Attune NxT Flow Cytometer (ThermoFisher). HEK-293T cells were lifted from the plate using TrypLE (Gibco), and resuspended in Stain Buffer (BD). These experiments were conducted in triplicate transfections. Cells were gated for single cells using forward and side scatter, and then on cells expressing fluorescent eGFP. Next, mTagBFP2 fluorescence was measured to indicate the amount of un-recombined attD plasmids, and mCherry fluorescence was measured to indicate the amount of recombinant plasmid.

An experiment testing recombinases with matched and unmatched attD plasmids was performed similarly, following the above protocol for K562 cells. 3 days after transfection, cells were measured by flow cytometry on a BD Accuri C6 cytometer.

Landing pad cell line production. Landing pad LSR candidates were cloned into lentiviral plasmids under the expression of the strong pEF1a promoter, with their attB site in between the promoter and start codon, and with a 2A-EGFP fluorescent marker downstream the LSR coding sequence. Lentivirus production and spinfection of K562 cells were performed as follows: HEK-293T cells were plated on 6-well tissue culture plates. On each plate, 5×10⁵ HEK-293T cells were plated in 2 mL of DMEM, grown overnight, and then transfected with 0.75 μg of an equimolar mixture of the three third-generation packaging plasmids (pMD2.G, psPAX2, pMDLg/pRRE) and 0.75 μg of LSR vectors using 10 μl of polyethyleneimine (PEI, Polysciences #23966) and 200 μl of cold serum free DMEM. pMD2.G (Addgene plasmid #12259: RRID:Addgene_12259), psPAX2 (Addgene plasmid #12260; RRID:Addgene_12260), and pMDLg/pRRE (Addgene plasmid #12251; RRID:Addgene_12251). After 24 hours, 3 mL of DMEM was added to the cells, and after 72 hours of incubation, lentivirus was harvested. The pooled lentivirus was filtered through a 0.45-μm PVDF filter (Millipore) to remove any cellular debris. 1×10⁵ K562 cells were infected with the lentiviruses by spinfection for 2 hours at 1000×g at 33° C. Lentivirus doses of 50, 100, and 200 μl were used for each vector, in order to find a condition with low multiplicity of infection wherein each transduced cell would be likely to contain only a single integrated copy of the landing pad. Infected cells grew for 3 days and then infection efficiency was measured using flow cytometry to measure EGFP (BD Accuri C6); the dose that gave rise to 5-15% EGFP+cells was selected for each LSR for further experiments. Ten days later, these EGFP+cells were sorted into a 96-well plate with a single cell in each well, in order to derive clonal lines with a single landing pad location. Two weeks later, 4 clones for each LSR with a unimodal high EGFP expression level were selected for expansion and subsequent experiments.

Landing pad integration efficiency assay. Clonal landing pad lines were electroporated with the promoterless mCherry donor containing the matching attP at a dose of either 1000 or 2000 ng donor plasmid. At timepoints from 3-11 days post-electroporation, the cells were subjected to flow cytometry to measure mCherry (BD Accuri C6).

Pseudosite integration efficiency assay to measure integration percent into the WT genome. To determine the percentage of integration of attD donors into pseudosites in the human genome, attD sequences were cloned into a plasmid containing an Ef1a promoter followed by mCherry, and p2a self-cleaving peptide, and a puromycin resistance marker. 1.0×10⁶ K562 cells were electroporated in Amaxa solution (Lonza Nucleofector SF, program FF-120), with 3000 ng LSR plasmid and 2000 ng pseudosite attD plasmid. As a non-matching LSR control, 3000 ng of Bxb1 was substituted for the correct LSR plasmid. The cells were cultured between 2×10⁵ cells/mL and 1×10⁶ cells/mL for 2-3 weeks. 100 uL of each sample was run on the Attune NxT Flow Cytometer every 3-4 days to measure the mCherry signal. After 2-3 weeks, transiently transfected plasmid was nearly fully diluted out in the non-matching LSR control, and the efficiency of the LSR was determined by the difference in mCherry percentage between the non-matching LSR control and the experimental condition.

Integration site mapping assay to determine human genome integration specificity. Utilizing the same protocol as above, K562s were electroporated with LSR and pseudosite attD plasmids. After 5 days in culture, puromycin was added to the media at 1 ug/mL. The cells were cultured for 1.5 more weeks, and then the gDNA was harvested using the Quick-DNA Miniprep Kit (Zymo) and quantified by Qubit HS dsDNA Assay (Thermo). A modified version of the UDiTaS sequencing assay was used as described in Giannoukos et al. BMC Genomics 19, 212 (2018), and Danner, 2020 Protocols.io.(doi(dot)org(backslash)10.17504(backslash)protocols.io.7k2hkye). Tn5 was purified and stored at 7.5 mg/mL. Adaptors were assembled by combining 50 uL of 100 uM top and bottom strand, heating to 95° C. for 2 minutes, and slowly ramping down to 25° C. over 12 hours. Next, the transposome was assembled by combining 85.7 uL of Tn5 transposase with 14.3 uL pre-annealed oligos, and incubated for 60 minutes at room temperature. Tagmentation was performed by adding 150 ng gDNA, 4 μL of 5×TAPS-DMF (50 mM TAPS NaOH, 25 mM MgCl2, 50% v/v DMF (pH 8.5) at 25° C.), 3 uL assembled transposome, and water for a 20 uL final reaction volume. The reaction was incubated at 55° C. for 10-15 minutes and then purified with Zymo DNA Clean and Concentrator-5. The tagmented products were run on Agilent Bioanalyzer HS DNA kit to confirm average fragment size of ˜2 kb. Next, PCR was performed with the outer primers for 12 cycles using 12.5 uL Platinum Superfi PCR Master Mix (Thermo), 1.5 uL of 0.5M TMAC, 0.5 uL of 10 uM outer nest GSP primer, 0.25 uL of 10 uM outer i5 primer, 9 ul of tagmented DNA, and 1.25 uL of DMSO. After Ampure XP 0.9× bead clean-up, a second PCR with the inner next primers, was performed for 18 cycles. The PCR contained 25 uL Platinum Superfi Master Mix (Thermo), 3 uL 0.5M TMAC, 2.5 uL DMSO, 2.5 uL of 10 uM i5 primer, 5 μL of 10 uM i7 GSP primer, 10 uL of the purified 1 st round PCR product, and 2 uL water for a final reaction volume of 50 μL. The final library was size selected on a 2% agarose gel for fragments between 300-800 bases, gel extracted with the Monarch DNA Gel Extraction Kit (NEB), quantified with Qubit HS dsDNA Assay (Thermo) and KAPA Library Quantification Kit, fragment analyzed with Agilent Bioanalyzer HS DNA kit, and sequenced on a MiSeq (Illumina).

Computational analysis of integration site mapping sequence assay. Snakemake workflows were constructed and used to analyze NGS data for the UDiTaS pseudosite sequencing assay. First, stagger sequences (filler sequences added for better discrimination of samples during sequencing) were added to primers were removed using custom python scripts. Next, fastp was used to trim nextera adaptors from reads and to remove reads with low PHRED scores. Next, reads were aligned to both the human genome (GRCh38) and a donor plasmid sequence containing the LSR-specific attD sequence in single-end mode using BWA. Reads were analyzed individually using custom python scripts to identify 1) if they aligned to the donor plasmid, human genome, or both, 2) whether or not the reads began at the predicted primer, and 3) whether or not the pre-integration attachment site was intact. Reads were then filtered to only include those reads that mapped to both the donor plasmid and the human genome, those that began at the primer site, and those that did not have an intact attD sequence (if this could be determined from the length of a particular read). This filtered read set was then aligned in paired-end mode to the human genome using default settings in BWA MEM. Alignments with a mapping quality score less than 30 were removed, along with supplementary alignments and paired read alignments with an insert size longer than 1500 bp. The samtools markdup tool was used to remove potential PCR duplicates and identify unique reads for downstream analysis. Next, MGEfinder was used to extract clipped end sequences from reads aligned to the human genome and generate a consensus sequence of the clipped ends, which represent the crossover from the human genome into the integrated attD sequence. Using custom python scripts, k-mers of length 9 base pairs were extracted from these consensus sequences and compared with a subsequence of the attD plasmid extending from the original primer to 25 bp after the end of the attD attachment site. If there were no shared 9-mers, the candidate was discarded. Otherwise, consensus sequences were clipped to begin at the primer site, and these consensus sequences were then aligned back to the original attD subsequence using the biopython local alignment tool. Two aligned portions were extracted—the full local alignment of the consensus sequence to the attD (called the “full local alignment”), and the longest subset of the alignment that included no ambiguous bases and no gaps (called the “contiguous alignment”). To filter a final set of true insertion sites, only sites with at least 80% nucleotide identity shared between the consensus sequence and the attD subsequence in either the full local alignment or the contiguous alignment were kept. Finally, only sites with a crossover point within 15 base pairs of the predicted dinucleotide core were kept.

This approach could precisely predict integration sites, but errors in sequencing reads led to some variability in this prediction. To account for this, integration sites were combined into integration “loci” by merging all sites that were within 500 base pairs of each other, using bedtools. This approach would merge integration events that occurred at the same site but in opposite orientations, for example. When pooling reads across biological or technical replicates, these loci were also merged if they overlapped. When measuring the relative frequency of insertion across different loci, all uniquely aligned reads (deduplicated using samtools markdup) found within each locus were counted. These were then converted into percentages for each locus by dividing by the total number of unique reads aligned to all integration loci.

Target site motifs for different LSRs could be determined from precise predictions of dinucleotide cores for all integration sites. For each integration locus, only one integration site was chosen if there were multiple, and integration sites with more reads supporting them were prioritized. Up to 30 base pairs of human genome sequence around the predicted dinucleotide core were extracted using bedtools, choosing the forward or reverse strand depending on the orientation of the integration. All such target sites, or a subset of these target sites if desired, were then analyzed for conservation at each nucleotide position using the ggseqlogo package in R.

Phylogenetic tree construction. Representative amino acid sequences of each quality-controlled 50% identity LSR cluster were used to construct the phylogenetic tree. LSRs were aligned using MAFFT in G-INS-i mode, and IQ-TREE was then used to generate a consensus tree using 1000 bootstrap replicates and automatic model selection.

### Example 1

### Systematic Identification of Recombinases and Predicted Attachment Sites Revealed Site-Specific and Multi-Targeting/Transposable Clades

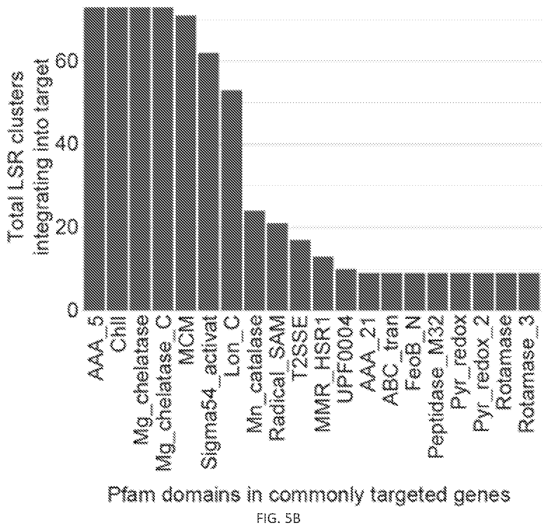

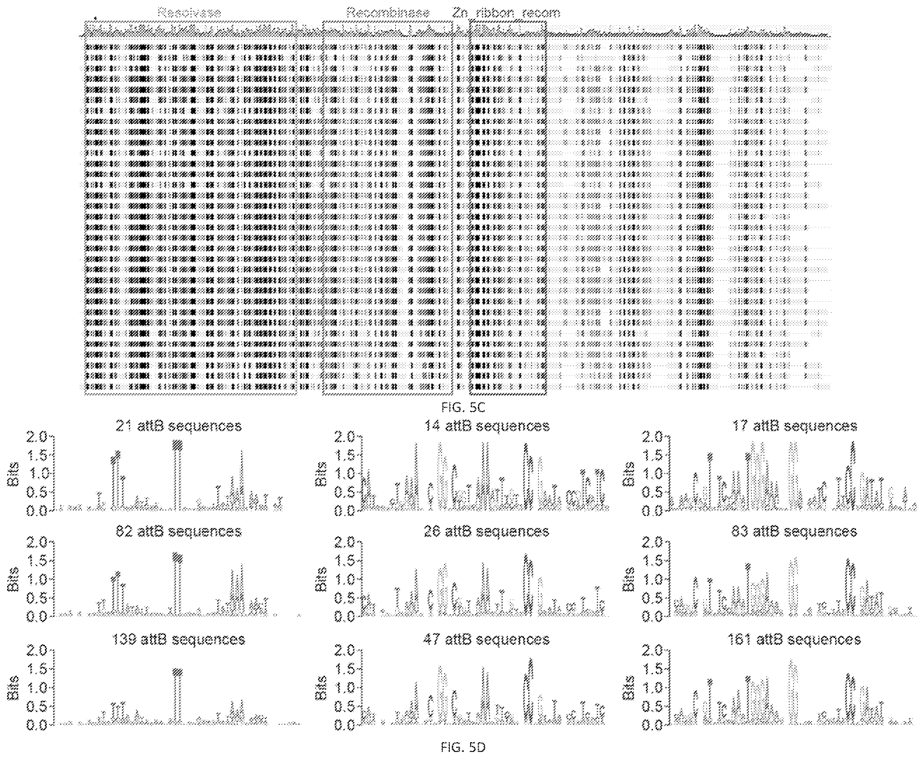

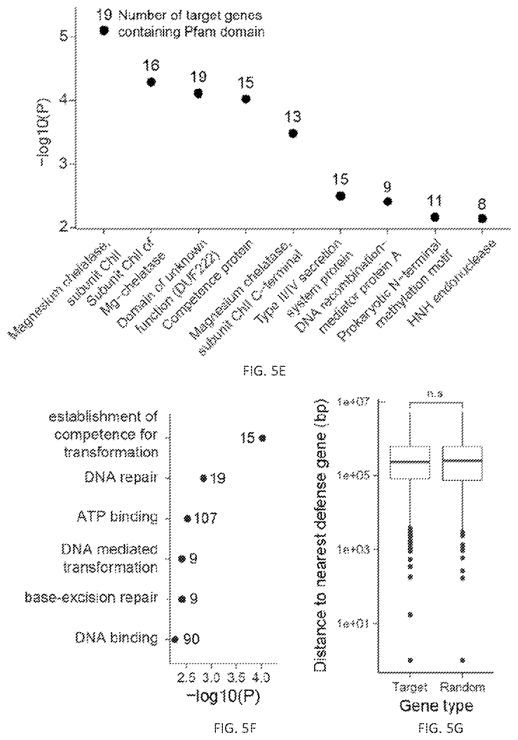

LSRs such as Bxb1 and PhiC31 catalyze an integration reaction that recombines two DNA sequences at specific attachment sites, referred to as attP (the DNA sequence found in the phage) and attB (the DNA sequence found in the bacteria). Using a comparative genomics approach built to identify precise boundaries of integrative elements (FIG. 1A), thousands of LSRs were identified in public databases of clinical and environmental bacterial isolate genomes. Once LSRs were identified, closely related genomes (average nucleotide identity (ANI)>95%) that lacked a given LSR were searched for, and used the previously developed bioinformatics tool MGEfinder (Durrant et al. (2020) Cell Host & Microbe 28(5): 767, incorporated herein by reference in its entirety) was used to align whole genomes with and without LSRs, thus allowing identification of the integrated prophage or mobile genetic element sequences (FIG. 1A). The boundaries of these predicted sequences represent the attL and attR sites that form when attP recombines with attB (FIG. 1A, box), and flank the integrated prophage genome or mobile genetic element containing the LSR. By using this approach on 194,585 bacterial isolate genomes, 12,638 candidate LSRs were identified and their original attP and attB attachment sites were reconstructed. After applying various quality control filters, and clustering protein sequences at 50% identity, the final dataset of LSR-attachment site predictions included 1,081 LSR clusters recovered from genomes belonging to 20 host phyla (FIG. 5A), indicating good representation of published bacterial assemblies.

To predict the site-specificity of candidate LSRs using only the constructed database, the network of LSRs and associated attachment sites were inspected, and LSRs from a diverse set of 20 host phyla were recovered (FIG. 5A), indicating good representation of published bacterial assemblies. Integration patterns across LSR clusters were compared. If many distantly-related LSRs appeared to target similar integration sites, it is likely that these LSRs would be site-specific. Conversely, if LSR clusters targeted many distinct integration sites, then they would be “multi-targeting,” meaning that they either had relaxed sequence specificity or they evolved to target sequences that occurred at multiple different sites in their host organisms. Target similarity was measured by mapping the attB integration sites to nearby ORF predictions, allowing attB sites to be grouped by the ORF sequence, referred to as a “target gene.” The protein sequences of these target genes were then clustered at 50% amino acid identity to further group more distantly related integration sites together. Clustering by target gene rather than attB sequence alone facilitated use of protein homology rather than DNA homology, grouping more distantly related target sites.

For each LSR cluster, the number of associated target gene clusters were estimated and visualized on the phylogenetic tree of representatives of each LSR cluster at the amino acid level. LSRs were binned into two groups: “Site-specific integrases” or “Multi-targeting integrases” (FIG. 1B). 82.8-88.3% of LSR clusters were predicted to be site-specific, or to have intermediate site-specificity, where the total number of unique target genes is 1, 2, or 3, depending on strictness of criteria used. One clade emerged of many multi-targeting LSRs, or those predicted to have to integrate into more than 3 target protein families, suggesting that this was an evolved strategy inherited from a single ancestor. This clade correlated strongly with DUF4368, a Pfam domain of unknown function (FIG. 5A), and that it includes previously characterized LSRs in the Tnd-like transposase subfamily (H. Wang and Mullany 2000 Journal of Bacteriology 182 (23): 6577-83; Adams et al. 2004 Molecular Microbiology 53 (4): 1195-1207, each incorporated herein by reference in its entirety).

Many examples of distantly related LSRs targeted the same gene clusters (FIGS. 1D and 1E). In FIG. 1D, an example of a network of diverse LSR clusters that primarily target a single gene cluster, a gene with homologs annotated as an ATP-dependent protease/Mg(2+) chelatase family protein/ComM-like protein, containing predicted Pfam domains ChlI (Subunit ChlI of Mg-chelatase), Mg_chelatase (Magnesium chelatase, subunit ChlI), and Mg_chelatase_C (Magnesium chelatase, subunit ChlI C-terminal) is shown. Homologs of this particular gene are one of the most commonly targeted genes (FIG. 5E), being targeted by 12.4% of all predicted site-specific integrases (FIG. 5B). FIG. 1E shows an example of a diverse set of LSRs that were found to target a single conserved site, the CDS sequence of a Prolyl isomerase. Upon aligning the LSR candidates that targeted this site, the DNA-binding Resolvase, Recombinase, and Zn_ribbon_recom domains were found to be much more conserved than the C-terminus, which is not believed to play an important role in DNA-binding (FIG. 5C). A more comprehensive enrichment in DNA competence genes and no enrichment within or near anti-phage defense genes (FIGS. 5E-5G)

FIG. 1G shows an example network of a multi-targeting LSR. Several multi-targeting LSRs have large numbers of associated attB target sites, which allowed inference of their sequence specificity computationally from the database. As shown in FIG. 1H, a single multi-targeting integrase was found to integrate into 21 distinct sites. Aligning target sites revealed a conserved TT dinucleotide core, with 5′ and 3′ ends enriched for T and A nucleotides, respectively. This suggested that this particular example most likely has relaxed sequence specificity overall, with the TT central dinucleotide being the most important feature for integration. Other examples of multi-targeting LSRs with distinct target site motifs are shown in FIG. 5D, including several with more complex motifs than the AT-rich one shown in FIG. 1H.

### Example 2

### Characterization of Landing Pad LSRs

One valuable application for LSRs in biotechnology is specific delivery of genetic cargo to an introduced site or so-called ‘landing pad’ that is not present elsewhere in the target genome. An ideal landing pad LSR is highly specific for an attB that does not exist in a target genome, but can efficiently integrate once the attB is installed.

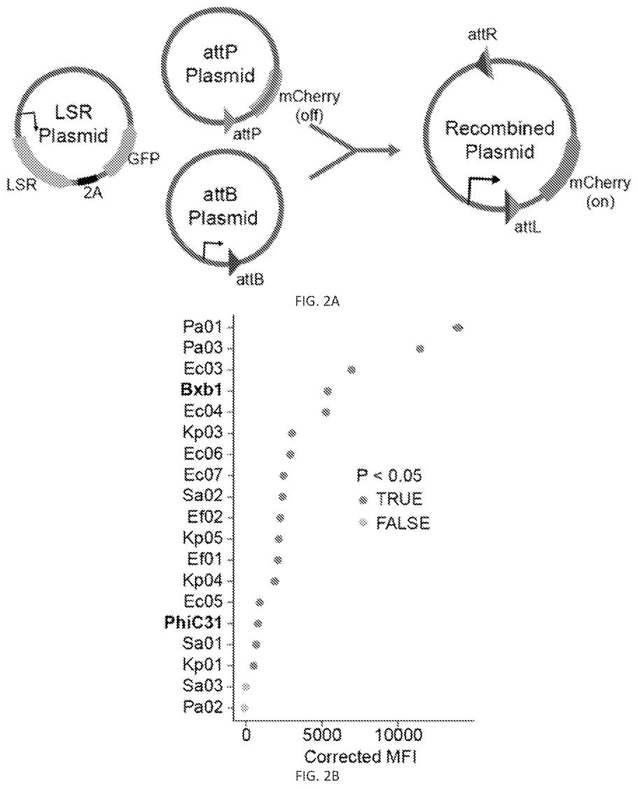

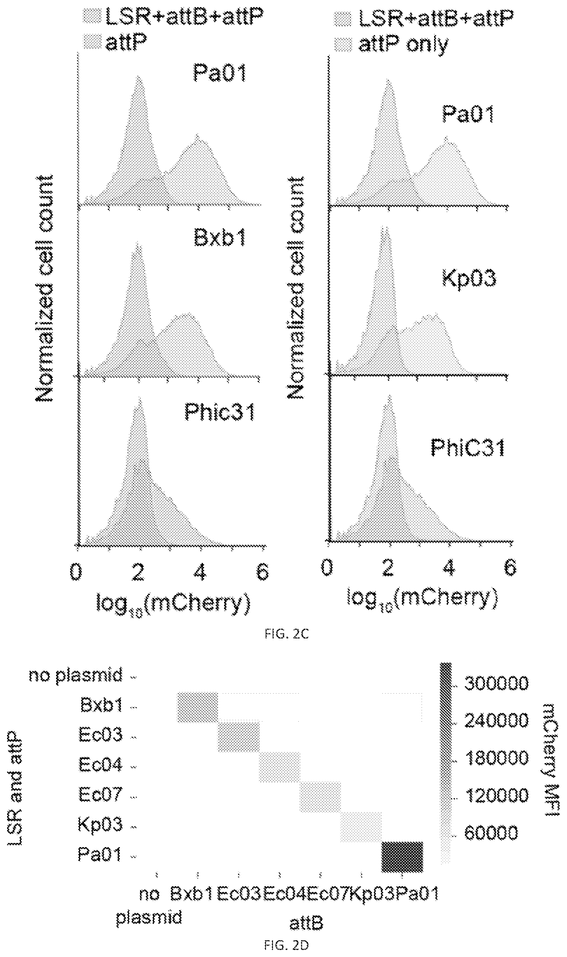

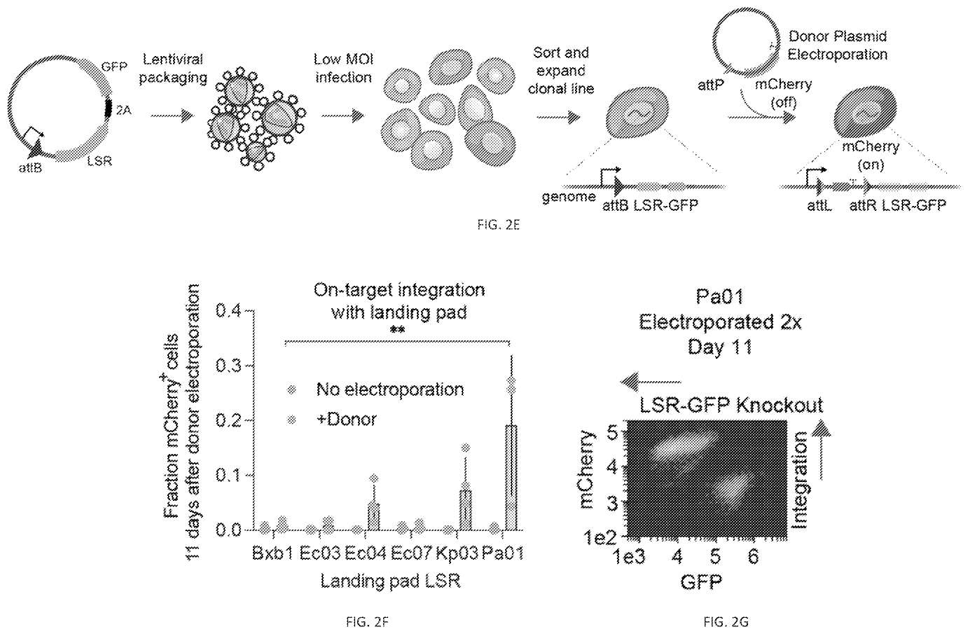

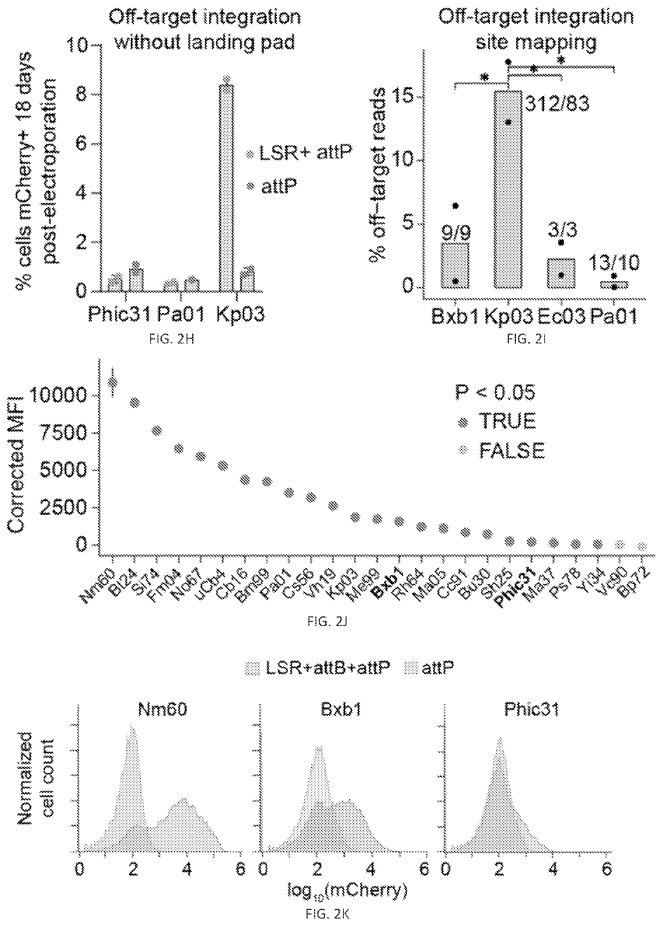

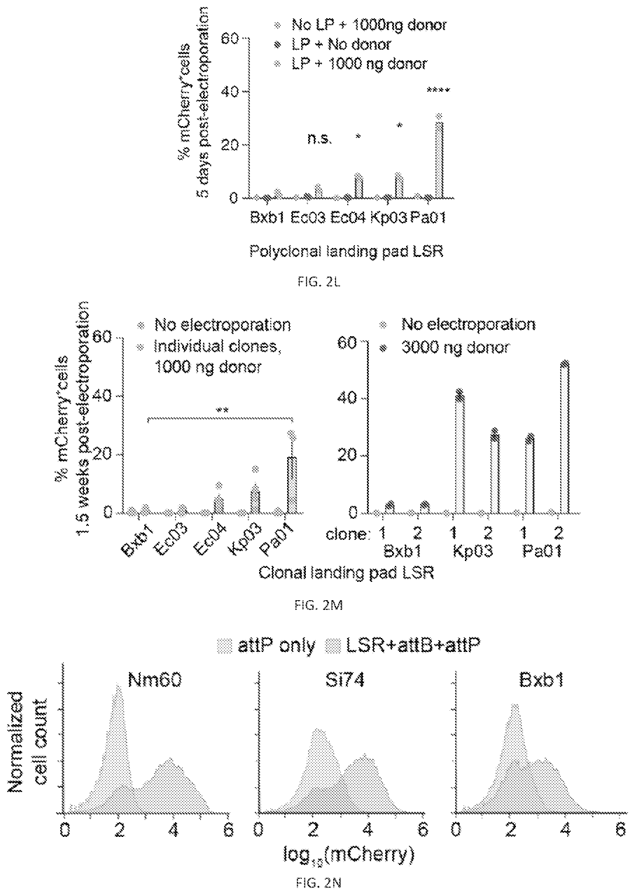

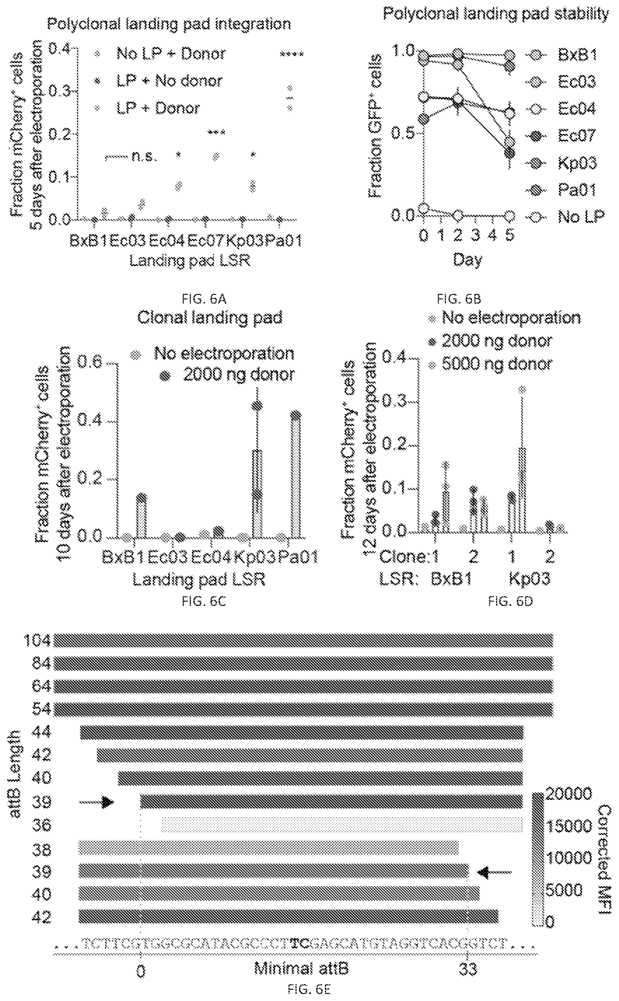

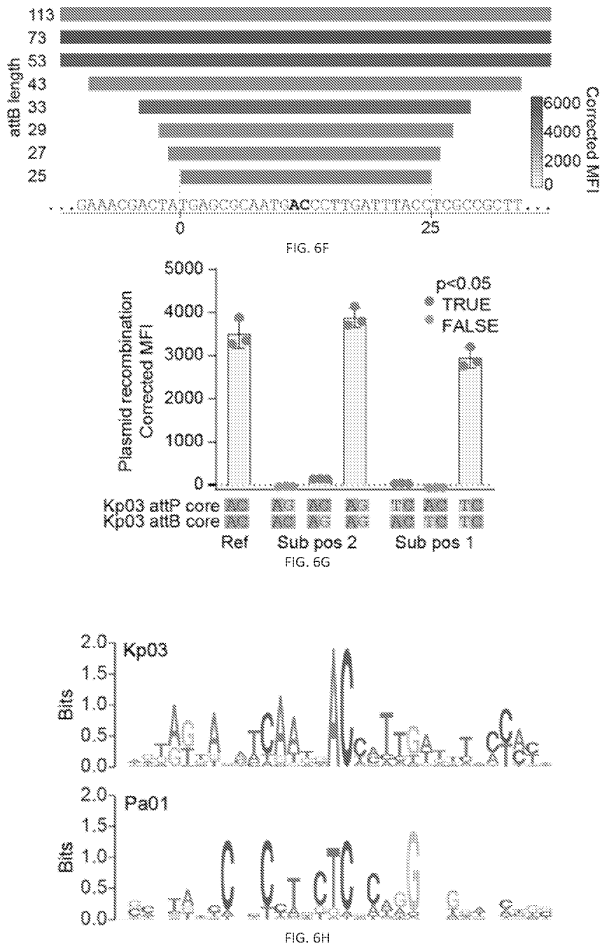

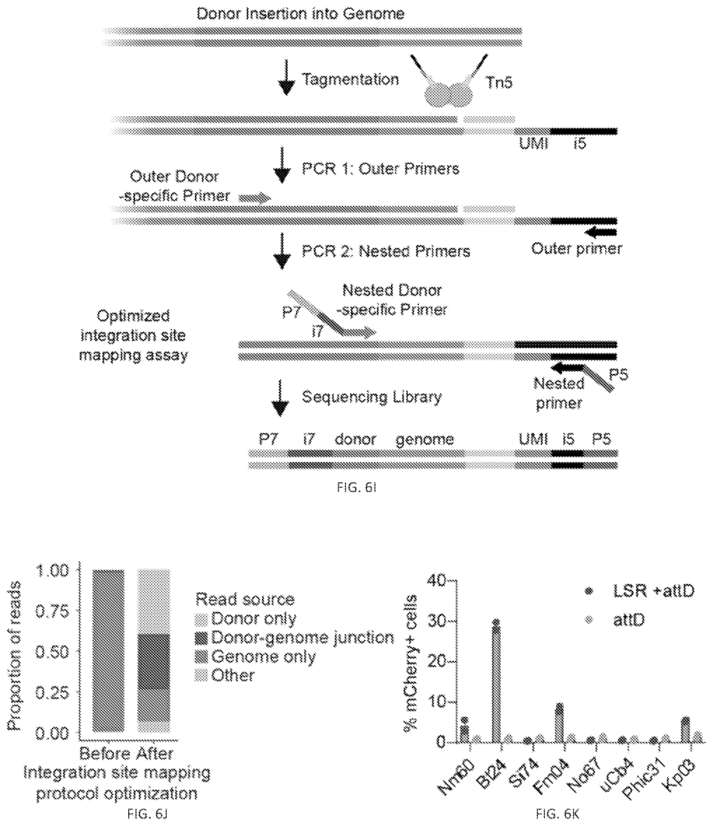

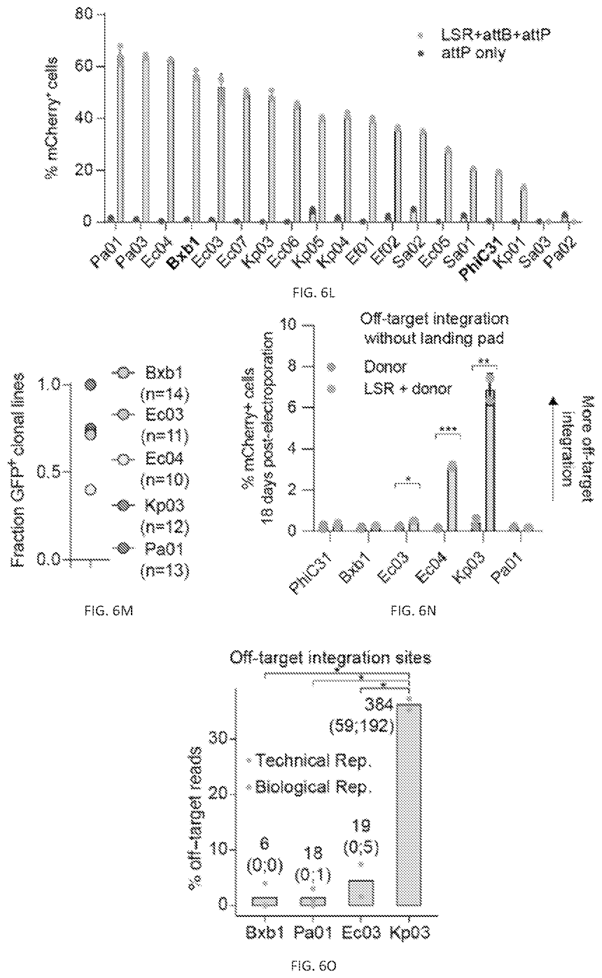

Using previously identified MGEs for LSRs (Durrant et al. (2020) Cell Host & Microbe 28(5): 767, incorporated herein by reference in its entirety), a set of 17 LSR candidates with evidence for site-specificity was curated as an initial proof of concept. To validate that these recombinases were active in mammalian cells, an inter-plasmid recombination assay was developed in HEK293FT cell by synthesizing three plasmids: one for expression of the human codon-optimized LSRs, and separate plasmids containing their putative attP and attB sequences (FIG. 2A). In this plasmid recombination assay, the attP plasmid contains a promoterless mCherry, which gains a promoter upon recombination with the attB plasmid resulting in fluorescent protein expression that can be read by flow cytometry. In the initial set of 17 candidates, 15 candidates were identified with greater mCherry+MFI values than attD-only controls (one-tailed t-test, P<0.05), demonstrating functional recombination (FIGS. 2B, 2C, and 6L). In comparison to positive controls, 13 candidates had greater mCherry+MFI than PhiC31, and 3 had greater mCherry+MFI than Bxb1. For a subset of LSRs, attachment site orthogonality was tested using the assay with different attachment site combinations, and it was found that they are highly specific and orthogonal to each other (FIG. 2D).

Integration into attB-containing landing pads that were pre-installed in the human genome were also tested (FIG. 2F). A construct containing an Ef1a promoter, attB, the matching LSR and GFP were integrated into the genome of K562 cells via high MOI lentivirus, resulting in a polyclonal population of cells likely to have the landing pad in different chromosomal locations in each cell. Upon successful integration of the promoterless mCherry donor into the landing pad, mCherry is expressed while GFP is knocked out. Using this landing pad assay, 5 of the new LSRs were found to integrate into human genome with measurable efficiency and Ec04, Ec07, Kp03, and Pa01 were significantly more efficient than BxB1 (FIGS. 6A and 2L). The stability of these polyclonal landing pads expressing LSR-GFP was assessed by flow cytometry over time and for some landing pads such as Ec07 and Ec03, the majority of cells lost GFP expression, suggesting the landing pad was transcriptionally silenced or genetically unstable (FIG. 6B). The LSRs can function on human chromosomal DNA and Kp03 and Pa01 emerged as top candidates in terms of efficiency.

Landing pad integration may be most useful when the landing pad is known to be at a single genomic site in all cells. To develop single position landing pad lines, landing pad LSR-GFP construct was integrated via low MOI lentivirus, resulting in a single copy of the landing pad per cell. Clonal cell lines which should contain a single landing pad site were then sorted, expanded, and electroporated with the attP-mCherry donor plasmid. Using this landing pad assay, four integrase candidates (Ec03, Ec04, Kp03, and Pa01) were tested and Pa01 performed better than Bxb1 in terms of the percentage of cells that were stably fluorescent after 11 days (FIG. 2F). With a tripled donor DNA dose (3000 ng), Pa01 reached 52% efficiency, while Bxb1 remained at 3% integration (FIG. 2M). In one Pa01 experiment, electroporating cells with donor plasmids twice increased integration efficiency to over 70% (FIG. 2G). Differences in efficiencies were reduced at higher donor DNA doses (FIGS. 6C-6D), suggesting variable integration kinetics for the different LSRs.

Previous characterization of the Bxb1 attB identified a sequence as short as 38 bp as being necessary for integration, but the computational pipeline conservatively predicted 100 bp attB sequences initially. A minimum 33 base pair attB for efficient Pa01 recombination was determined, but efficient recombination for Kp03 was seen down to a 25 base pair attB (FIG. 6F). At short lengths, the attachment sites can be easily installed during cloning and cell engineering through a variety of methods.

Efficient landing pads could be especially useful for multiplex gene integration, which could be achieved by using several of LSRs in parallel, given that they do not operate on each other's attachment sites (FIG. 2D). Interestingly, other well-studied LSRs Bxb1 and PhiC31 contain a modular dinucleotide core in their attachment sites that can be changed to enable orthogonal integrations (Ghosh, Kim, and Hatfull 2003 Molecular Cell 12 (5): 1101-11, incorporated herein by reference in its entirety), such that the same LSR can be applied to direct multiple cargoes to specific landing pads that differ by their core dinucleotides. The ability to substitute core dinucleotides was tested using the plasmid recombination assay for one of the LSRs, Kp03 (FIG. 6G). Changing either nucleotide of the dinucleotide core in one attachment site dramatically reduced integration efficiency, and subsequently changing the other attachment site to match the first restored integration efficiency. This suggested that this LSR could be used to orthogonally integrate different cargos at up to 10 different attachment site landing pads.

The specificity of these LSRs was tested by transfecting attP-pEF1a-mCherry donors with or without co-transfected LSR into wildtype K562 cells and measuring mCherry expression 18 days later, by which point episomal donor plasmid is no longer detectable. Pa01 showed no evidence of mCherry integration above background, while Kp03 did have elevated mCherry+fluorescence, suggesting it has off-target pseudosites (FIG. 2H). To identify these sites, the UDiTaS™ genome-wide single-sided PCR-based sequencing assay was modified for use as an LSR integration site mapping assay. After optimizing this assay, the proportion of target-derived reads was increased from 1.6% to 73.2% (FIG. 6J). This assay was first performed on the landing pad cell lines, allowing estimation of the percentage of off-target integrations relative to integrations on-target integrations (FIG. 2I).

This assay detected off-target integration for all LSRs, including Bxb1 (3.48%+/−2.98%, 9 unique reads across 9 integration loci) and Pa01 (0.47%+/−0.46%, 13 unique reads across 10 loci), but Kp03 had significantly more than the others at 15.5%+/−2.43%, with 312 unique reads detected across 83 different loci, confirming a relatively high percentage of off-target integrations. Wild-type cells that were transfected with Kp03 and Pa01 were sequenced using the integration site mapping assay at high coverage, and 79 off-target genome integration loci were detected for Pa01, and 2,415 off-target integration loci were detected for Kp03. From these integration sites, the target site motifs targeted by these LSRs were identified, and the motifs showed conservation at the dinucleotide core and flanking sequence, indicating that these are bona fide integrations rather than random plasmid integrations (FIG. 2H). Together, these results establish Pa01 as a more efficient and comparably specific landing pad LSR in comparison to BxB1.

A second batch of 21 LSRs were selected from the database, prioritizing those with low BLAST similarity between their attB/P sites and the human genome, and applying stringent quality thresholds. 17 out of 21 (81%) of them were functional in the plasmid recombination assay, providing validation of the computational pipeline for identifying functional candidates. Promisingly, 16 candidates had higher mCherry+MFI values than PhiC31, and 11 candidates had higher MFI values than Bxb1 (FIG. 2J). The integration fluorescence assay in wild-type cells using top candidates identified 3 with low percentage off-target integrations (FIG. 6K), with Si74 being a top candidate with favorable performance in terms of both plasmid recombination efficiency and off-target integrations (FIG. 2K).

### Example 3

Genome-Targeting LSRs Integrate into Human Genome at Predicted Target Sites

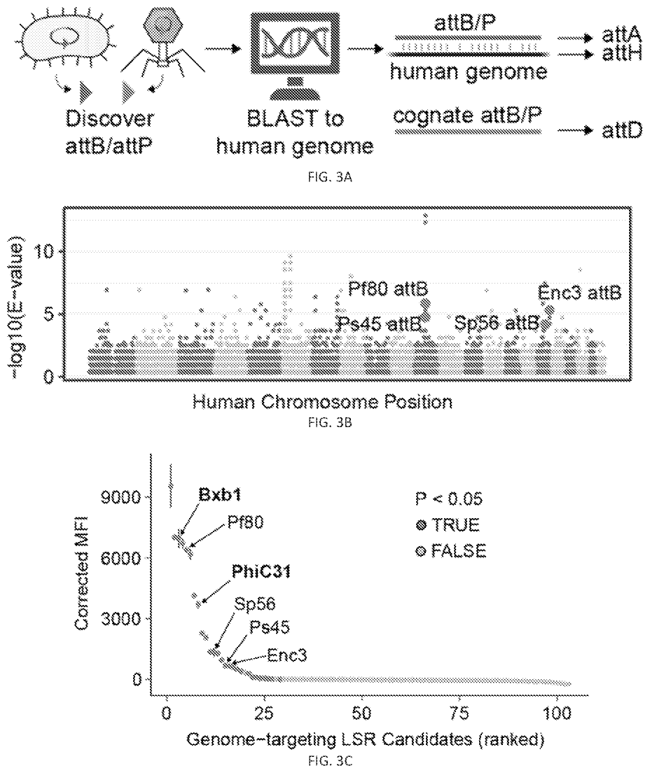

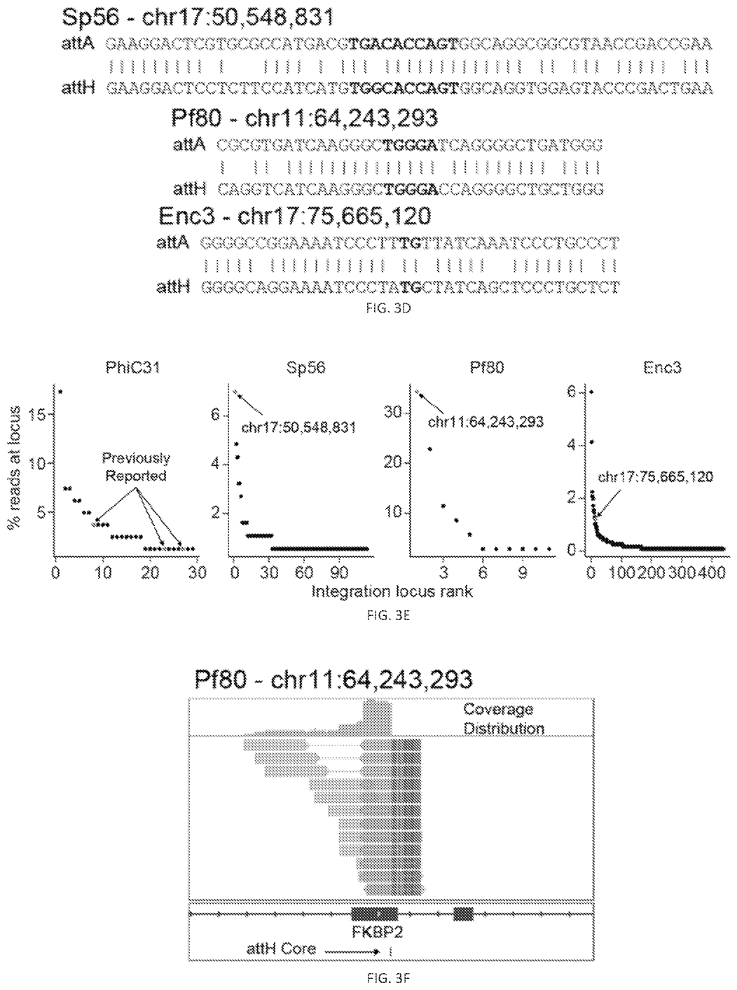

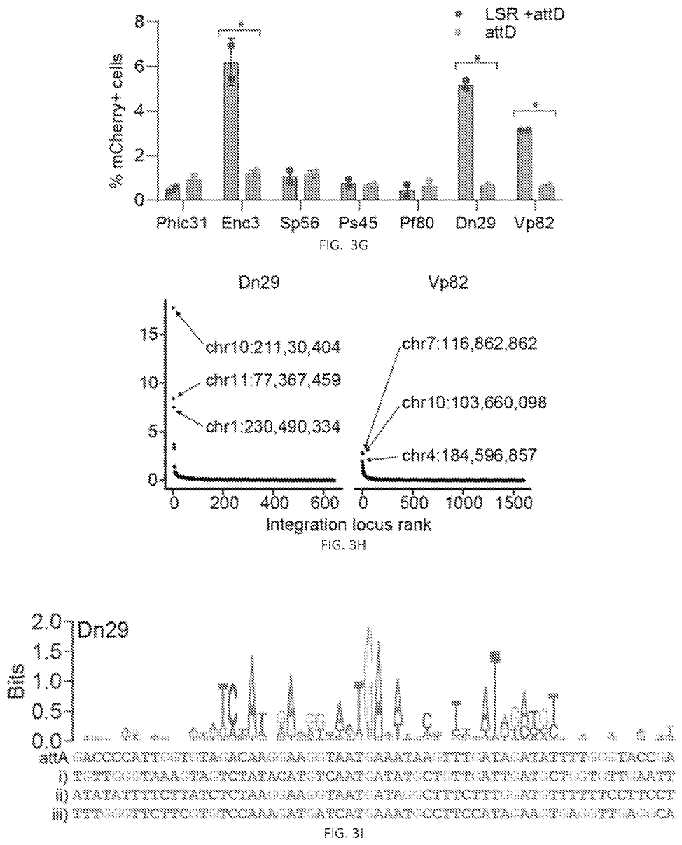

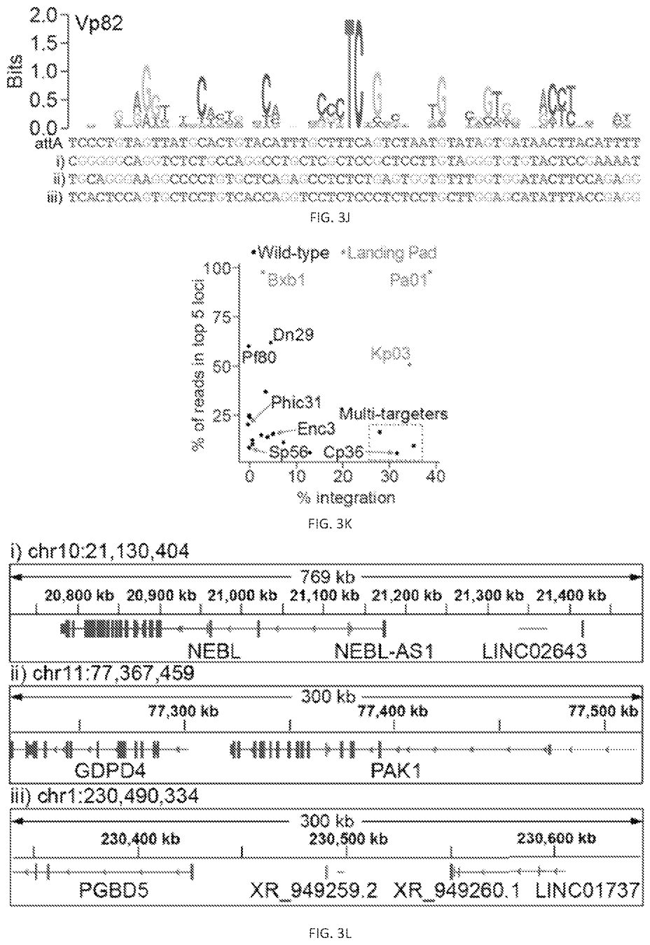

A particularly useful LSR would be one that integrates directly into only one, or very few, pseudosites in safe locations in the human genome and does so with appreciable efficiency. Historically, LSRs with pseudosites such as that for PhiC31 had to be experimentally discovered by transfecting the LSR into human cells and searching for the integration sites. While effective in demonstrating proof of concept, this approach has not yielded highly efficient and specific human genome-targeting LSRs. BLAST was used to search all attB/P sequences against the GRCh38 human genome assembly (FIG. 3A) and 856 LSRs with a highly significant match for at least one site were identified in the human genome (BLAST E-value <1e-3, FIG. 3B). Many of these LSR-attachment site predictions did not meet the quality control thresholds, but BLAST match quality was prioritized when selecting candidates, and 103 LSRs of varying quality were synthesized, attP and attB sites were renamed according to their BLAST hits, with the attachment site that matched the human genome being renamed to attA (acceptor), and the other being renamed to attD (donor). The predicted target site in the human genome was renamed attH (human) (FIGS. 3A and 3D).

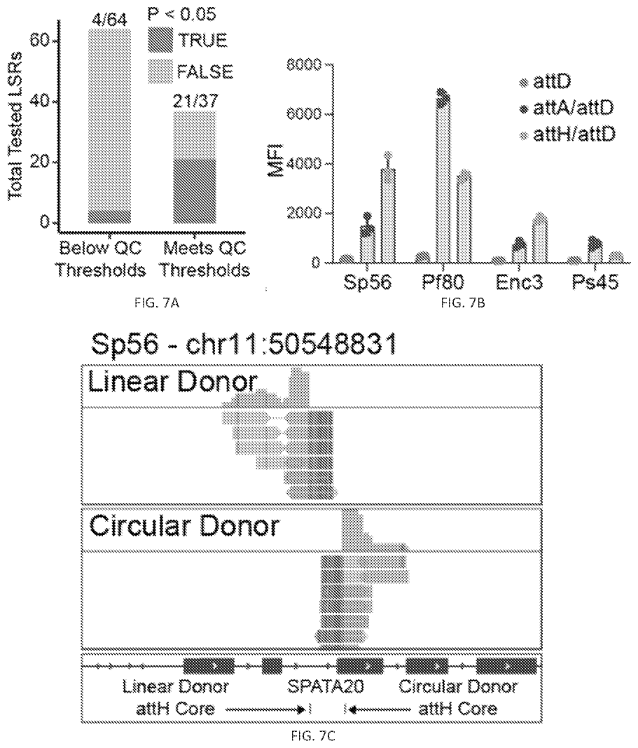

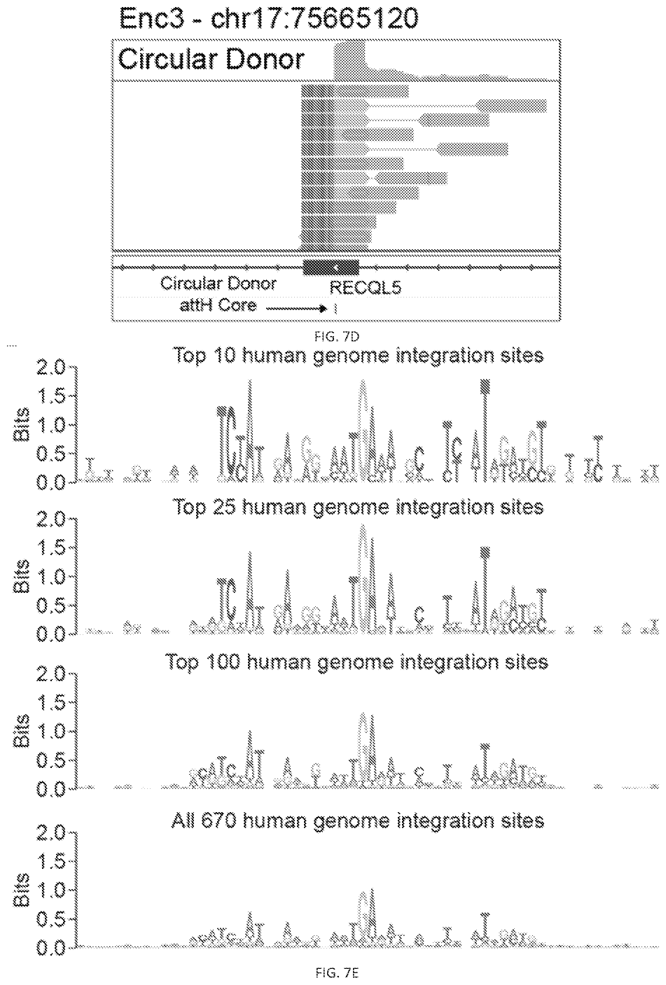

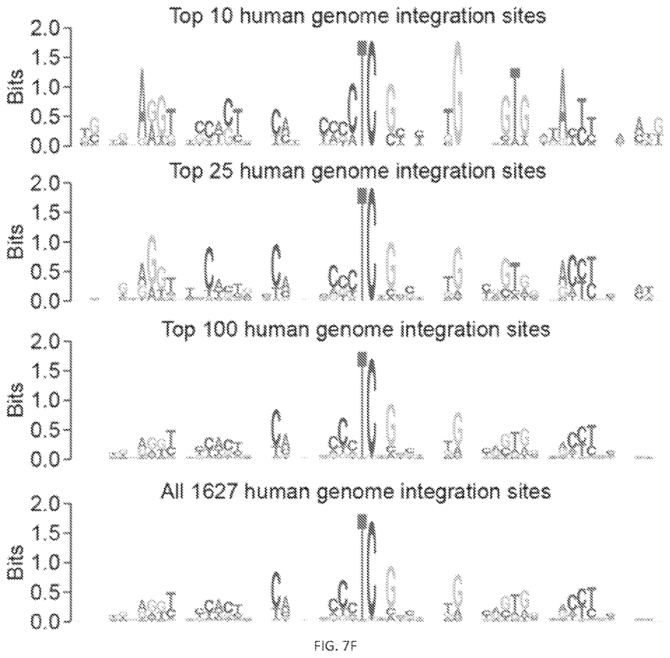

All 103 candidates were tested in the plasmid recombination assay, and 27 candidates recombined at predicted attachment sites (one-tailed t-test, P<0.05; FIG. 3C), with 4 out of 64 (6.25%) low-quality candidates recombining as predicted, and 21 out of 37 (56.75%) high-quality candidates recombining as predicted (FIG. 7A). In subsequent batches of genome-targeting candidates, only high-quality LSR-attachment site predictions were utilized, which included 201 unique LSRs with attachment sites that significantly matched sites in the human genome (BLAST E-value <1e-3).

To determine if these LSRs could target the chromosomes directly in human K562 cells, another plasmid recombination assay was performed, replacing the native attA with the human pseudosite (or attH) instead of the native attachment site and found 4 of the candidates recombined with their predicted attH: Sp56, Pf80, Ps45, and Enc3 (FIG. 3D). This was followed by a human genome integration assay and an integration site mapping assay. Several integration sites were detected for all of these candidates when using both circular donor plasmids and linear PCR amplicons. For Sp56 and Pf80, the integration sites with the most unique reads (presumed to be the most frequently target loci) across experiments were the target sites that were predicted by BLAST alignments, an exon of SPATA20 and an exon of FKBP2, respectively (FIG. 3E). For Enc3, the predicted target site had the 12th most reads of all loci with detected integrations. Ps45 had detected reads at the predicted target site in one experiment, but coverage was too low to estimate relative specificity. Examples of reads from the integration site mapping assay aligned to the predicted site are shown in FIGS. 3F, 7C and 7D. These four examples demonstrated that candidates can be selected prior to experimental validation based on BLAST similarity to the human genome, and that 4 out of 27 (14.8%) functional candidates tested were able to recombine with the predicted site.

Of these four candidates, Pf80 had the highest predicted specificity, with 34.3% of unique reads mapping to the predicted target site, an exon of the gene FKBP2 at position 64,243,293 on chromosome 11 (FIG. 3F). But in the efficiency assay, Pf80, Sp56, and Ps45 did not have mCherry+fluorescence above background, suggesting low overall efficiency (FIG. 3G). Enc3 had the highest efficiency of these candidates, with 6% of cells being mCherry+ at day 18 after transfection. Other genome-targeting candidates were subsequently tested, Dn29 and Vp82, had 4.5% and 2.5% mCherry+cells in the efficiency assay, respectively, but no integrations were detected at their predicted target sites in the integration site mapping assay (FIGS. 3G-3H). Dn29 had relatively high specificity, with 17.4% of unique reads mapping to its top target site, and 33.0% of unique reads mapping to the top three target sites. An analysis of Dn29 and Vp82 integration sites revealed distinct sequence profiles of their targets, which may inform future efforts to engineer and optimize these candidates (FIGS. 3I-3J and 7E-7F). Several of these candidates outperform PhiC31 in terms of efficiency and specificity, with Dn29 having a favorable mix of both, making them promising genome-targeting candidates. An ideal genome-targeting LSR would integrate with robust efficiency in a site-specific manner. The genome-targeting candidates tested exhibited varying levels of efficiency, with Enc3 and Dn29 in particular having significantly higher efficiency (6% and 5%, respectively) than PhiC31 or Pf80 (both <1%: FIG. 3G). For Dn29, 61.9% of integrations occurred in just the top 5 target sites, which were found in intronic or intergenic regions (FIGS. 3K-3L).

### Example 4

Multi-Targeting LSRs Directly Integrate DNA into the Human Genome

An LSR is considered to be a good multi-targeting candidate if it has relaxed specificity requirements, if it appears in the multi-targeting clade (FIG. 1B), and/or if it has DUF4368, a Pfam domain that was found to correlate with the multi-targeting clade (FIG. 5A).

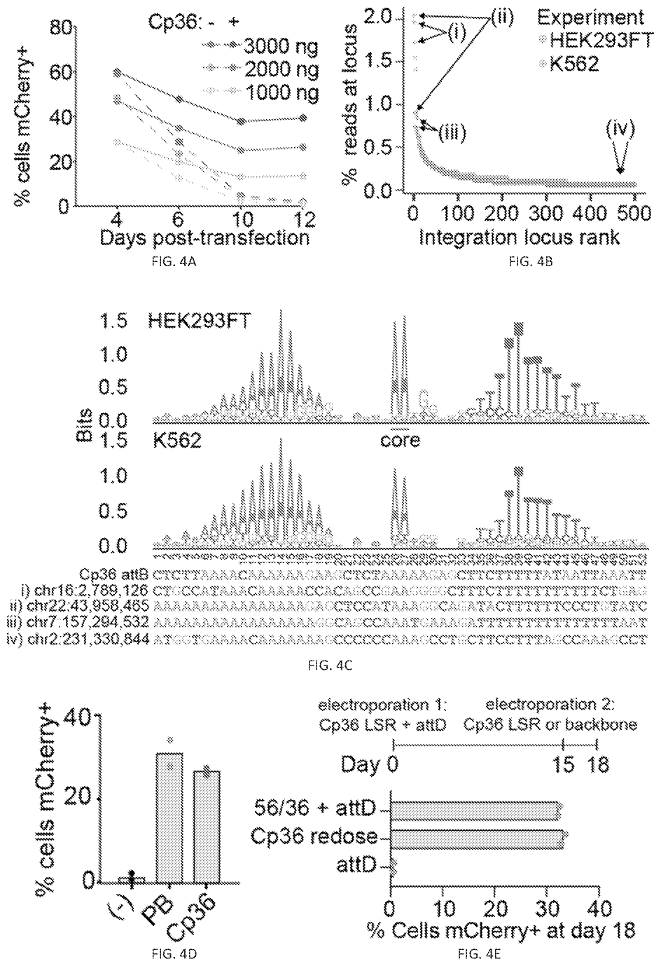

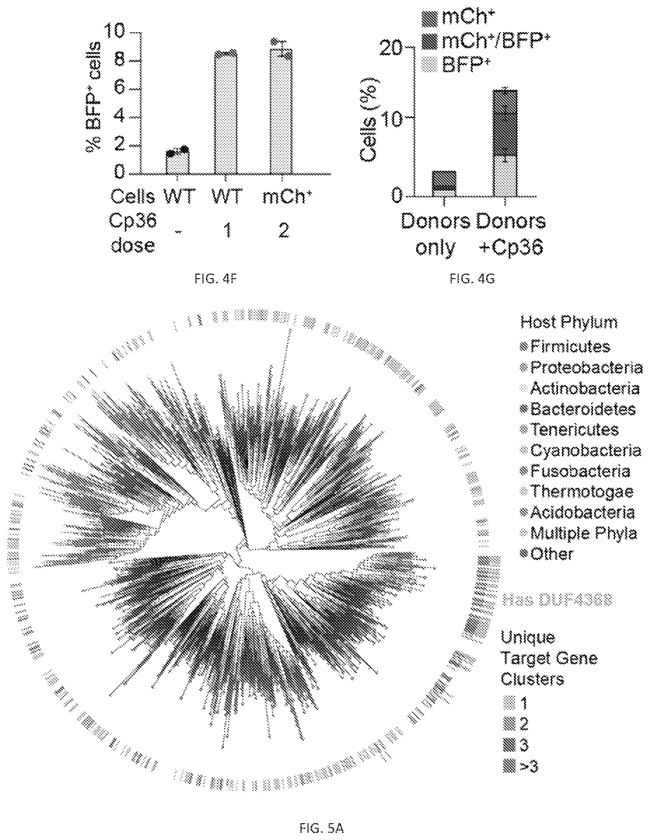

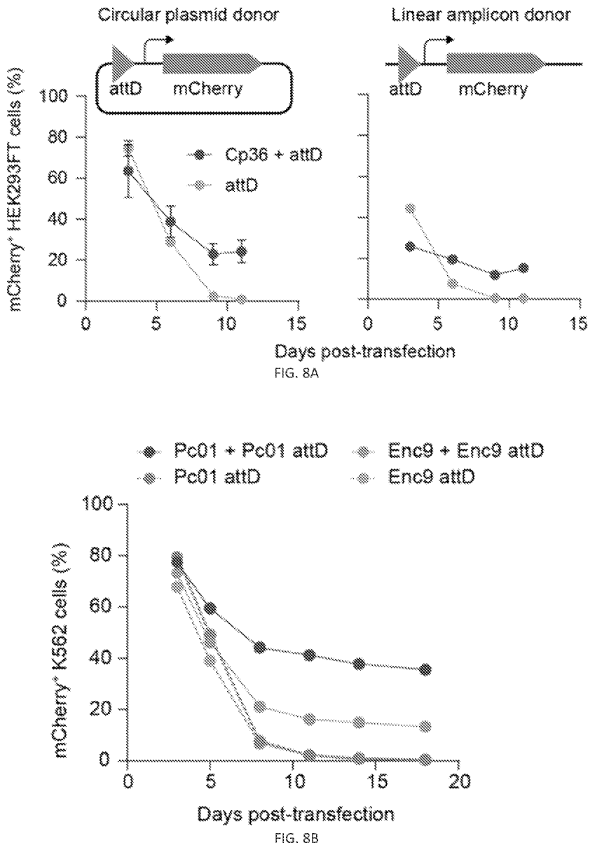

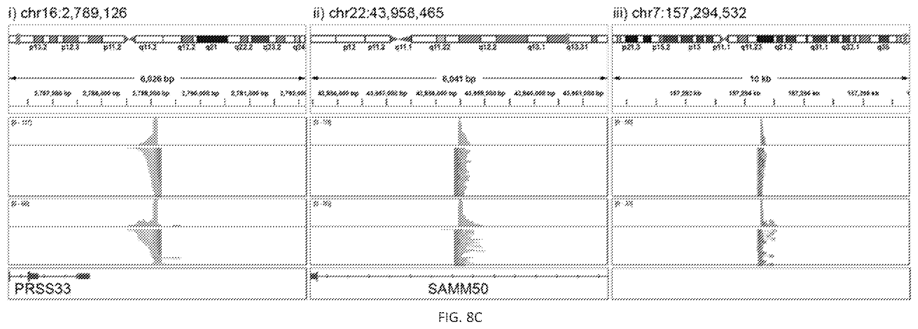

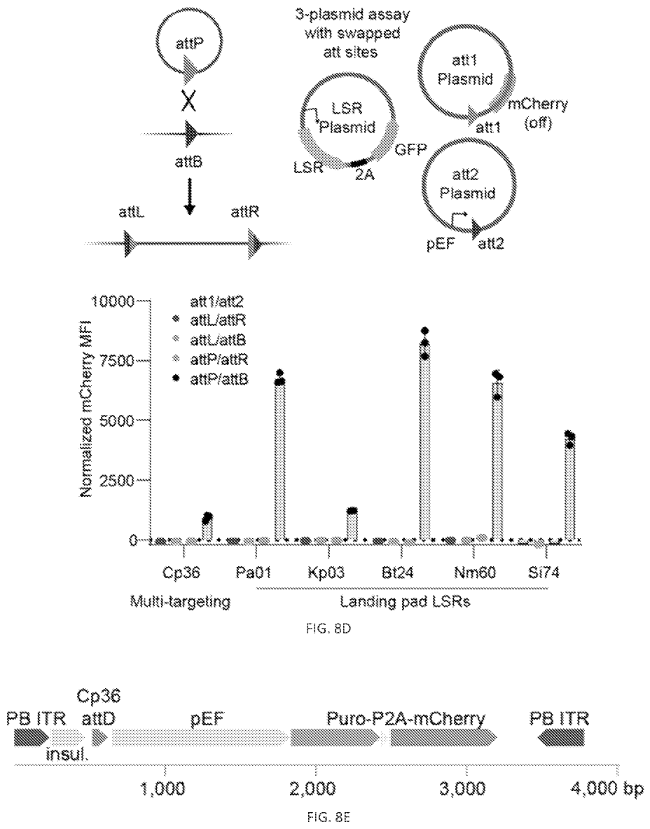

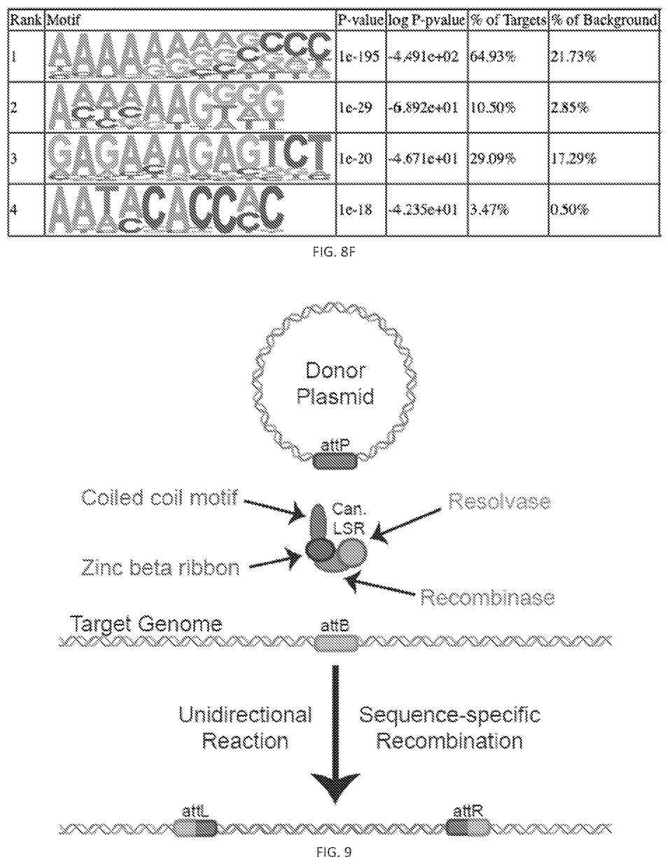

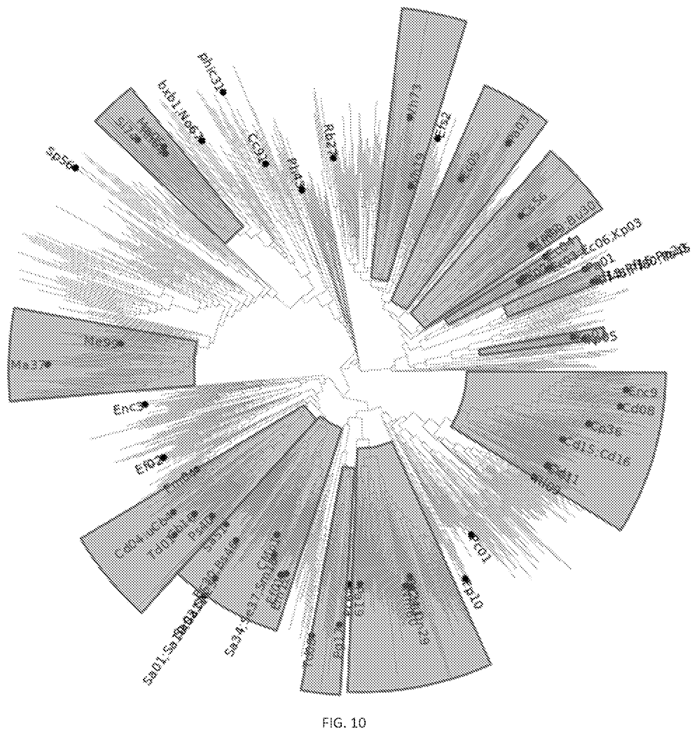

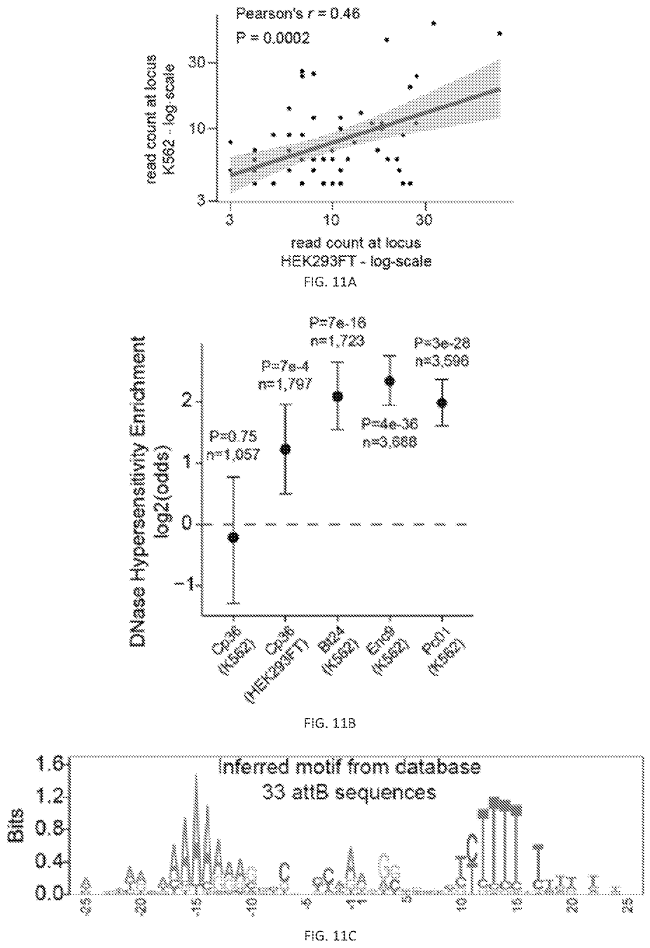

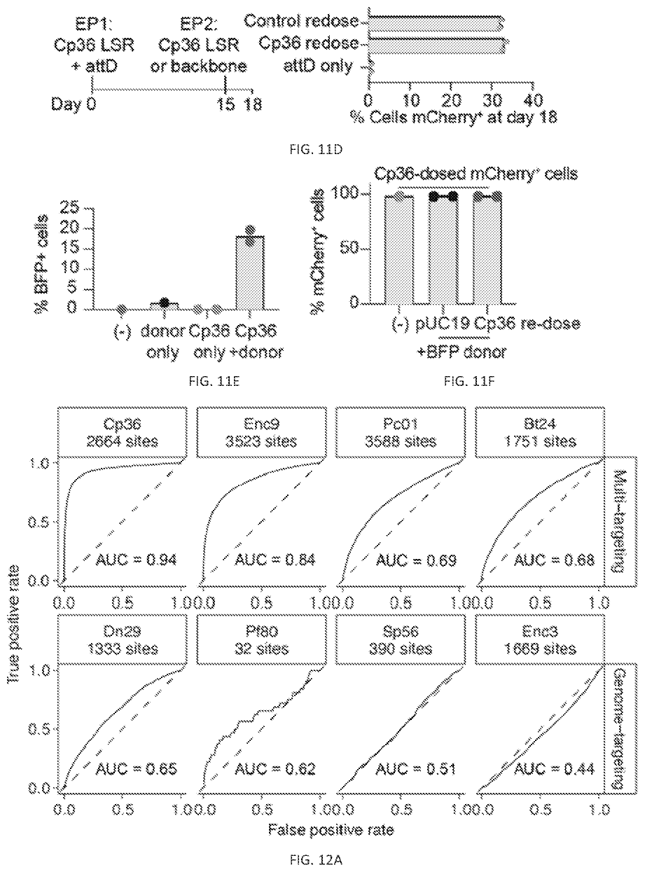

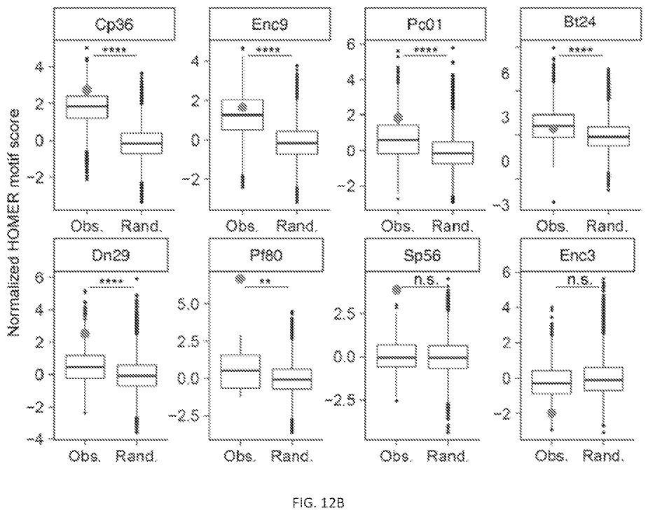

One such multi-targeting LSR found in Clostridium perfringens, named Cp36, was characterized. This LSR is 544 amino acids in length, and it contains a predicted DUF4368 domain at its C-terminus. This LSR can integrate an mCherry donor cargo into the genome of K562 cells at up to 40% efficiency without pre-installation of a landing pad or antibiotic selection (FIG. 4A). This high level of integration efficiency was verified in HEK293FT cells, utilizing both plasmid DNA and linear PCR amplicons as the donor cargo (FIG. 8A). Using the integration site mapping assay, over 2000 unique integration sites were found, with a strong bias toward specific sites (FIGS. 4B and 8C). The locus with the most integration events, chr1:101,429,889 (w.r.t. GRCh38), was the target of approximately 2% of all integration events. There was high concordance across the two cell types, with a jaccard similarity of 20% among the top 100 sites in both cell types, and a jaccard similarity of 17.8% among the top 200 sites. The number of unique reads at the top 61 sites that were found in both cell types is highly correlated (Pearson's r=0.45, P=0.0002. FIGS. 8D and 11A), suggesting that the relative efficiency of integration at these sites is quite consistent across cell types.

Using these precise prediction of human integration sites, a sequence motif targeted by Cp36 was reconstructed (FIG. 4C). This sequence motif is composed of an A-rich 5′ region, followed by the AA dinucleotide core, followed by a 3′ T-rich region. The natural attB in the C. perfringens genome and three commonly targeted human genome target sites, it was clear that the three human genome integration sites were close matches for the motif. One target site having low efficiency integration in both cell types was also a good match for the motif, although with shorter stretches of A and T nucleotides on the 5′ and 3′ ends. The poly-A and poly-T flanks matched previous descriptions of the natural attB for TndX, a previously characterized LSR that is 35.4% identical to Cp36 at the amino acid level.

To compare the efficiency of Cp36 to the PiggyBac (PB) transposase, a commonly used tool for delivering DNA cargos at random into TTAA tetranucleotides found in a target genome, a plasmid construct was designed that included a Cp36 attD (donor attachment site), PB ITR sequences, and an mCherry reporter (FIG. 8E). Cp36 performed at similar efficiencies to PB (FIG. 4D). Cp36 catalyzed uni-directional integration like other site-specific LSRs (FIGS. 4E, 8F and 8G), whereas PB has been shown to be bi-directional, resulting in both excision and local hopping of cargo upon PB redosing.

To test if Cp36 could be re-used to integrate a second gene, a pure population of mCherry+cells was generated via Cp36-mediated integration and puromycin selection, and re-electroporated with Cp36 and a donor containing BFP. After 13 days, 9% of the cells were double positive (mCherry+ and BFP+) (FIGS. 4F and 11E), without any reduction in mCherry (FIG. 11F), demonstrating delivery of a second gene without loss of the first cargo. Further, it was found that simultaneous delivery of Cp36 with both mCherry and BFP fluorescent reporter donors resulted in stable populations expressing both markers (FIG. 4G), suggesting that Cp36 could be used to generate cells with multi-part genetic circuits in a single transfection.

Additionally, two other orthologs (Pc01 and Enc9) were found in the database that also functioned as multi-targeters in human cells with efficiencies of 13% and 35% (FIG. 8B). These results reveal the existence of a subset of LSRs, not previously tested in eukaryotic cells, with highly efficient, unidirectional integration activity and longer targeted DNA motifs (≥20 bp) compared to lentivirus or transposase systems (2-4 bp).

### Example 5

### Biological Role of LSR Target Genes

Genes that were targeted and disrupted upon LSR integration could indicate an evolved strategy for LSR-carrying MGEs. Pfam domains that were enriched among target genes were identified (FIG. 5E). Enriched domains were found in Magnesium chelatases, Competence proteins, Type II/IV secretion system proteins, and HNH endonucleases, among others. Gene ontology (GO) pathway analysis of the target genes identified six pathways that were significantly enriched (FDR<0.1: FIG. 5F). Notably, the GO term “establishment of competence for transformation” (GO:0030420) was the most significantly enriched pathway with 15 target gene clusters being annotated with this term. Among these target genes was the ComK transcription factor and other ComG operon proteins, suggesting that disrupting competence and DNA transformation is a common strategy for LSR-carrying MGEs. Reasoning that LSRs may have also evolved to target host anti-phage defense systems upon integration, relevant genomes were annotated using DefenseFinder and genes that occurred in or near these identified systems were searched. Some defense genes that were targeted by integrases, including CRISPR spacer acquisition gene cas2, CASCADE complex helicase cas3, Type I restriction modification enzymes. Hachiman defense gene hamA, and a UvrD-like helicase gene were identified. However, defense genes were rarely targeted by LSRs, and no enrichment of target genes was found near defense genes, suggesting this is not a common strategy (FIG. 5G). These findings support an evolved strategy adopted by LSR-carrying MGEs that limits further horizontal gene transfer primarily through disruption of competence.

### Example 6

### Post Hoc Identification of Human Genome Integration

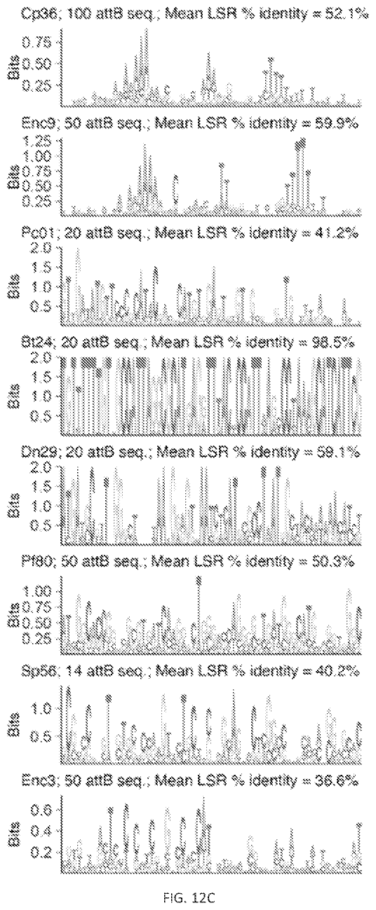

A post hoc analysis of the genome-targeting and multi-targeting candidates in this study was performed to determine how feasible a motif-based search would be. Starting with each experimentally characterized candidate, sequence motifs were built by iteratively adding natural attB sequences of the next most closely related LSR ortholog, only adding additional attB sequences if they were 95% identical or less to already selected attB sequences. Motifs of 20, 50 and 100 such attB sequences were built. Then these motifs were searched against the experimentally observed human integration sites, and approximately 30.000 randomly selected human genome sequences. Next, these sequences were iterated across motif score cutoffs and the true positive rate and the false positive rate were calculated at each cutoff, generating a ROC curve (FIG. 12A). For each LSR, the motif with the greatest AUC was selected.

Sequence motifs belonging to the multi-targeting candidates performed quite well, with AUC values ranging from 0.94 for the Cp36 motif to 0.68 for the Bt24 motif. For the genome-targeting candidates the performance of the sequence motifs varied, ranging in AUC values from 0.65 for Dn29 to 0.44 for Enc3. All of these motifs assigned significantly higher scores to observed integration sites than randomly selected controls, except for Sp56 and Enc3, which did not differ significantly (Wilcoxon rank-sum test; P<0.0001 for Cp36, Enc9, Pc01, Bt24, and Dn29, P<0.01 for Pf80, P>0.05 for Sp56 and Enc3). Despite the relatively poor performance of the Pf80 motif and the Sp56 motif, they did assign the highest motif scores to the most frequently targeted human genome integration sites, suggesting that there is predictive value to their database-derived sequence motifs (FIG. 12B). Upon visual inspection of the motifs a variety of patterns were seen, with Cp36 and Enc9 motifs having the characteristic AT rich motifs typical of many multi-targeting LSRs, and others such as Dn29 and Bt24 having less variation and less well-defined boundaries (FIG. 12C).

These results suggest that there is value in taking a motif-based sequence search when prioritizing multi-targeting and genome-targeting candidates. The potential targeting profile of multi-targeters could be better understood prior to experimental validation, as with Cp36 and Enc9, and genome-targeting candidates could be selected based on those that have high, outlier motif matches that could indicate higher specificity, such as for Pf80. The difference in performance between motifs may be explained by the different selection pressures placed on multi-targeting and single-targeting LSRs, where multi-targeting LSRs are more likely to maintain their relaxed sequence specificity across larger evolutionary distances due to a greater abundance of possible target sites, leading to more accurate sequence motifs. These results could also have been influenced by the efficiency of the LSR in human cells or epigenetic modifications such as those that influence chromatin accessibility (FIG. 8H).

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Table 1:
Landing Pad Integrases

| | attP | attB | Protein |
|---|---|---|---|
| | sequence | sequence | sequence |
| | SEQ ID | SEQ ID | SEQ ID |
| LSR | NOs: | NOs: | NOs: |
| Sh25 | 1184 | 1216 | 1 |
| Si74 | 1185 | 1217 | 2 |
| Bm99 | 1186 | 1218 | 3 |
| Me99 | 1187 | 1219 | 4 |
| Ma37 | 1188 | 1220 | 5 |
| Nm60 | 1189 | 1221 | 6 |
| Cc91 | 1190 | 1222 | 7 |
| Vh19 | 1191 | 1223 | 8 |
| Cs56 | 1192 | 1224 | 9 |
| Bt24 | 1193 | 1225 | 10 |
| No67 | 1194 | 1226 | 11 |
| Fm04 | 1195 | 1227 | 12 |
| Bu30 | 1196 | 1228 | 13 |
| Ma05 | 1197 | 1229 | 14 |
| Rh64 | 1198 | 1230 | 15 |
| Cb16 | 1199 | 1231 | 16 |
| uCb4 | 1200 | 1232 | 17 |
| Ec03 | 1201 | 1233 | 18 |
| Ec04 | 1202 | 1234 | 19 |
| Ec05 | 1203 | 1235 | 20 |
| Ec06 | 1204 | 1236 | 21 |
| Ec07 | 1205 | 1237 | 22 |
| Ef01 | 1206 | 1238 | 23 |
| Ef02 | 1207 | 1239 | 24 |
| Kp01 | 1208 | 1240 | 25 |
| Kp03 | 1209 | 1241 | 26 |
| Kp04 | 1210 | 1242 | 27 |
| Kp05 | 1211 | 1243 | 28 |
| Pa01 | 1212 | 1244 | 29 |
| Pa03 | 1213 | 1245 | 30 |
| Sa01 | 1214 | 1246 | 31 |
| Sa02 | 1215 | 1247 | 32 |

Table 2:
Genome-Targeting Integrases

| | attD | attA | Protein |
|---|---|---|---|
| | sequence | sequence | sequence |
| | SEQ ID | SEQ ID | SEQ ID |
| LSR | NOs: | NOs: | NOs: |
| Pf13 | 1248 | 1282 | 33 |
| Td08 | 1249 | 1283 | 34 |
| Se37 | 1250 | 1284 | 35 |
| Ct03 | 1251 | 1285 | 36 |
| Ps40 | 1252 | 1286 | 38 |
| Sa10 | 1253 | 1287 | 39 |
| Td01 | 1254 | 1288 | 40 |
| Enc3 | 1255 | 1289 | 41 |
| Fp10 | 1256 | 1290 | 42 |
| Ph43 | 1257 | 1291 | 43 |
| Sm18 | 1258 | 1292 | 44 |
| Pf80 | 1259 | 1293 | 46 |
| Bs46 | 1260 | 1294 | 47 |
| Pf48 | 1261 | 1295 | 48 |
| Rb27 | 1262 | 1296 | 49 |
| Sa51 | 1263 | 1297 | 50 |
| Bc30 | 1264 | 1298 | 51 |
| Cd04 | 1265 | 1299 | 52 |
| Sa34 | 1266 | 1300 | 54 |
| Pp20 | 1267 | 1301 | 55 |
| Efs2 | 1268 | 1302 | 57 |
| Pf15 | 1269 | 1303 | 58 |
| Ps45 | 1270 | 1304 | 59 |
| Sp56 | 1271 | 1305 | 60 |
| Dn29 | 1272 | 1306 | 61 |
| Vh73 | 1273 | 1307 | 62 |
| Em12 | 1274 | 1308 | 63 |
| Pc64 | 1275 | 1309 | 64 |
| Vp82 | 1276 | 1310 | 65 |
| CMp1 | 1277 | 1311 | 69 |
| Pa19 | 1278 | 1312 | 70 |
| Pg17 | 1279 | 1313 | 71 |
| Sall | 1280 | 1314 | 72 |
| El01 | 1281 | 1315 | 73 |

Table 3:
Multi-Targeting Integrases

| | attD | attA | Protein |
|---|---|---|---|
| | sequence | sequence | sequence |
| | SEQ ID | SEQ ID | SEQ ID |
| LSR | NOs: | NOs: | NOs: |
| Cp36 | 1316 | 1324 | 66 |
| Pc01 | 1317 | 1325 | 67 |
| Enc9 | 1318 | 1326 | 68 |
| Cd16 | 1319 | 1327 | 45 |
| Cd15 | 1320 | 1328 | 53 |
| Cd31 | 1321 | 1329 | 37 |
| Rl09 | 1322 | 1330 | 56 |
| Cd08 | 1323 | 1331 | 74 |
| | Table 4: | | |
| | LSR Protein | | |
| | sequence | attB sequence | attP sequence |
| | SEQ ID NOs: | SEQ ID NOs: | SEQ ID NOs: |
| | 88 | 1332 | 2413 |
| | 89 | 1333 | 2414 |
| | 90 | 1334 | 2415 |
| | 91 | 1335 | 2416 |
| | 92 | 1336 | 2417 |
| | 93 | 1337 | 2418 |
| | 94 | 1338 | 2419 |
| | 95 | 1339 | 2420 |
| | 96 | 1340 | 2421 |
| | 97 | 1341 | 2422 |
| | 98 | 1342 | 2423 |
| | 99 | 1343 | 2424 |
| | 100 | 1344 | 2425 |
| | 101 | 1345 | 2426 |
| | 102 | 1346 | 2427 |
| | 103 | 1347 | 2428 |
| | 104 | 1348 | 2429 |
| | 105 | 1349 | 2430 |
| | 106 | 1350 | 2431 |
| | 107 | 1351 | 2432 |
| | 108 | 1352 | 2433 |
| | 109 | 1353 | 2434 |
| | 110 | 1354 | 2435 |
| | 111 | 1355 | 2436 |
| | 112 | 1356 | 2437 |
| | 113 | 1357 | 2438 |
| | 114 | 1358 | 2439 |
| | 115 | 1359 | 2440 |
| | 116 | 1360 | 2441 |
| | 117 | 1361 | 2442 |
| | 118 | 1362 | 2443 |
| | 119 | 1363 | 2444 |
| | 120 | 1364 | 2445 |
| | 121 | 1365 | 2446 |
| | 122 | 1366 | 2447 |
| | 123 | 1367 | 2448 |
| | 124 | 1368 | 2449 |
| | 125 | 1369 | 2450 |
| | 126 | 1370 | 2451 |
| | 127 | 1371 | 2452 |
| | 128 | 1372 | 2453 |
| | 129 | 1373 | 2454 |
| | 130 | 1374 | 2455 |
| | 131 | 1375 | 2456 |
| | 132 | 1376 | 2457 |
| | 133 | 1377 | 2458 |
| | 134 | 1378 | 2459 |
| | 135 | 1379 | 2460 |
| | 136 | 1380 | 2461 |
| | 137 | 1381 | 2462 |
| | 138 | 1382 | 2463 |
| | 139 | 1383 | 2464 |
| | 140 | 1384 | 2465 |
| | 141 | 1385 | 2466 |
| | 142 | 1386 | 2467 |
| | 143 | 1387 | 2468 |
| | 144 | 1388 | 2469 |
| | 145 | 1389 | 2470 |
| | 146 | 1390 | 2471 |
| | 147 | 1391 | 2472 |
| | 148 | 1392 | 2473 |
| | 149 | 1393 | 2474 |
| | 150 | 139 | 2475 |
| | 151 | 1395 | 2476 |
| | 152 | 1396 | 2477 |
| | 153 | 1397 | 2478 |
| | 154 | 1398 | 2479 |
| | 155 | 1399 | 2480 |
| | 156 | 1400 | 2481 |
| | 157 | 1401 | 2482 |
| | 158 | 1402 | 2483 |
| | 159 | 1403 | 2484 |
| | 160 | 1404 | 2485 |
| | 161 | 1405 | 2486 |
| | 162 | 1406 | 2487 |
| | 163 | 1407 | 2488 |
| | 164 | 1408 | 2489 |
| | 165 | 1409 | 2490 |
| | 166 | 1410 | 2491 |
| | 167 | 1411 | 2492 |
| | 168 | 1412 | 2493 |
| | 169 | 1413 | 2494 |
| | 170 | 1414 | 2495 |
| | 171 | 1415 | 2496 |
| | 172 | 1416 | 2497 |
| | 173 | 1417 | 2498 |
| | 174 | 1418 | 2499 |
| | 175 | 1419 | 2500 |
| | 176 | 1420 | 2501 |
| | 177 | 1421 | 2502 |
| | 178 | 1422 | 2503 |
| | 179 | 1423 | 2504 |
| | 180 | 1424 | 2505 |
| | 181 | 1425 | 2506 |
| | 182 | 1426 | 2507 |
| | 183 | 1427 | 2508 |
| | 184 | 1428 | 2509 |
| | 185 | 1429 | 2510 |
| | 186 | 1430 | 2511 |
| | 187 | 1431 | 2512 |
| | 188 | 1432 | 2513 |
| | 189 | 1433 | 2514 |
| | 190 | 1434 | 2515 |
| | 191 | 1435 | 2516 |
| | 192 | 1436 | 2517 |
| | 193 | 1437 | 2518 |
| | 194 | 1438 | 2519 |
| | 195 | 1439 | 2520 |
| | 196 | 1440 | 2521 |
| | 197 | 1441 | 2522 |
| | 198 | 1442 | 2523 |
| | 199 | 1443 | 2524 |
| | 200 | 1444 | 2525 |
| | 201 | 1445 | 2526 |
| | 202 | 1446 | 2527 |
| | 203 | 1447 | 2528 |
| | 204 | 1448 | 2529 |
| | 205 | 1449 | 2530 |
| | 206 | 1450 | 2531 |
| | 207 | 1451 | 2532 |
| | 208 | 1452 | 2533 |
| | 209 | 1453 | 2534 |
| | 210 | 1454 | 2535 |
| | 211 | 1455 | 2536 |
| | 212 | 1456 | 2537 |
| | 213 | 1457 | 2538 |
| | 214 | 1458 | 2539 |
| | 215 | 1459 | 2540 |
| | 216 | 1460 | 2541 |
| | 217 | 1461 | 2542 |
| | 218 | 1462 | 2543 |
| | 219 | 1463 | 2544 |
| | 220 | 1464 | 2545 |
| | 221 | 1465 | 2546 |
| | 222 | 1466 | 2547 |
| | 223 | 1467 | 2548 |
| | 224 | 1468 | 2549 |
| | 225 | 1469 | 2550 |
| | 226 | 1470 | 2551 |
| | 227 | 1471 | 2552 |
| | 228 | 1472 | 2553 |
| | 229 | 1473 | 2554 |
| | 230 | 1474 | 2555 |
| | 231 | 1475 | 2556 |
| | 232 | 1476 | 2557 |
| | 233 | 1477 | 2558 |
| | 234 | 1478 | 2559 |
| | 235 | 1479 | 2560 |
| | 236 | 1480 | 2561 |
| | 237 | 1481 | 2562 |
| | 238 | 1482 | 2563 |
| | 239 | 1483 | 2564 |
| | 240 | 1484 | 2565 |
| | 241 | 1485 | 2566 |
| | 242 | 1486 | 2567 |
| | 243 | 1487 | 2568 |
| | 244 | 1488 | 2569 |
| | 245 | 1489 | 2570 |
| | 246 | 1490 | 2571 |
| | 247 | 1491 | 2572 |
| | 248 | 1492 | 2573 |
| | 249 | 1493 | 2574 |
| | 250 | 1494 | 2575 |
| | 251 | 1495 | 2576 |
| | 252 | 1496 | 2577 |
| | 253 | 1497 | 2578 |
| | 254 | 1498 | 2579 |
| | 255 | 1499 | 2580 |
| | 256 | 1500 | 2581 |
| | 257 | 1501 | 2582 |
| | 258 | 1502 | 2583 |
| | 259 | 1503 | 2584 |
| | 260 | 1504 | 2585 |
| | 261 | 1505 | 2586 |
| | 262 | 1506 | 2587 |
| | 263 | 1507 | 2588 |
| | 264 | 1508 | 2589 |
| | 265 | 1509 | 2590 |
| | 266 | 1510 | 2591 |
| | 267 | 1511 | 2592 |
| | 268 | 1512 | 2593 |
| | 269 | 1513 | 2594 |
| | 270 | 1514 | 2595 |
| | 271 | 1515 | 2596 |
| | 272 | 1516 | 2597 |
| | 273 | 1517 | 2598 |
| | 274 | 1518 | 2599 |
| | 275 | 1519 | 2600 |
| | 276 | 1520 | 2601 |
| | 277 | 1521 | 2602 |
| | 278 | 1522 | 2603 |
| | 279 | 1523 | 2604 |
| | 280 | 1524 | 2605 |
| | 281 | 1525 | 2606 |
| | 282 | 1526 | 2607 |
| | 283 | 1527 | 2608 |
| | 284 | 1528 | 2609 |
| | 285 | 1529 | 2610 |
| | 286 | 1530 | 2611 |
| | 287 | 1531 | 2612 |
| | 288 | 1532 | 2613 |
| | 289 | 1533 | 2614 |
| | 290 | 1534 | 2615 |
| | 291 | 1535 | 2616 |
| | 292 | 1536 | 2617 |
| | 293 | 1537 | 2618 |
| | 294 | 1538 | 2619 |
| | 295 | 1539 | 2620 |
| | 296 | 1540 | 2621 |
| | 297 | 1541 | 2622 |
| | 298 | 1542 | 2623 |
| | 299 | 1543 | 2624 |
| | 300 | 1544 | 2625 |
| | 301 | 1545 | 2626 |
| | 302 | 1546 | 2627 |
| | 303 | 1547 | 2628 |
| | 304 | 1548 | 2629 |
| | 305 | 1549 | 2630 |
| | 306 | 1550 | 2631 |
| | 307 | 1551 | 2632 |
| | 308 | 1552 | 2633 |
| | 309 | 1553 | 2634 |
| | 310 | 1554 | 2635 |
| | 311 | 1555 | 2636 |
| | 312 | 1556 | 2637 |
| | 313 | 1557 | 2638 |
| | 314 | 1558 | 2639 |
| | 315 | 1559 | 2640 |
| | 316 | 1560 | 2641 |
| | 317 | 1561 | 2642 |
| | 318 | 1562 | 2643 |
| | 319 | 1563 | 2644 |
| | 320 | 1564 | 2645 |
| | 321 | 1565 | 2646 |
| | 322 | 1566 | 2647 |
| | 323 | 1567 | 2648 |
| | 324 | 1568 | 2649 |
| | 325 | 1569 | 2650 |
| | 326 | 1570 | 2651 |
| | 327 | 1571 | 2652 |
| | 328 | 1572 | 2653 |
| | 329 | 1573 | 2654 |
| | 330 | 1574 | 2655 |
| | 331 | 1575 | 2656 |
| | 332 | 1576 | 2657 |
| | 333 | 1577 | 2658 |
| | 334 | 1578 | 2659 |
| | 335 | 1579 | 2660 |
| | 336 | 1580 | 2661 |
| | 337 | 1581 | 2662 |
| | 338 | 1582 | 2663 |
| | 339 | 1583 | 2664 |
| | 340 | 1584 | 2665 |
| | 341 | 1585 | 2666 |
| | 342 | 1586 | 2667 |
| | 343 | 1587 | 2668 |
| | 344 | 1588 | 2669 |
| | 345 | 1589 | 2670 |
| | 346 | 1590 | 2671 |
| | 347 | 1591 | 2672 |
| | 348 | 1592 | 2673 |
| | 349 | 1593 | 2674 |
| | 350 | 1594 | 2675 |
| | 351 | 1595 | 2676 |
| | 352 | 1596 | 2677 |
| | 353 | 1597 | 2678 |
| | 354 | 1598 | 2679 |
| | 355 | 1599 | 2680 |
| | 356 | 1600 | 2681 |
| | 357 | 1601 | 2682 |
| | 358 | 1602 | 2683 |
| | 359 | 1603 | 2684 |
| | 360 | 1604 | 2685 |
| | 361 | 1605 | 2686 |
| | 362 | 1606 | 2687 |
| | 363 | 1607 | 2688 |
| | 364 | 1608 | 2689 |
| | 365 | 1609 | 2690 |
| | 366 | 1610 | 2691 |
| | 367 | 1611 | 2692 |
| | 368 | 1612 | 2693 |
| | 369 | 1613 | 2694 |
| | 370 | 1614 | 2695 |
| | 371 | 1615 | 2696 |
| | 372 | 1616 | 2697 |
| | 373 | 1617 | 2698 |
| | 374 | 1618 | 2699 |
| | 375 | 1619 | 2700 |
| | 376 | 1620 | 2701 |
| | 377 | 1621 | 2702 |
| | 378 | 1622 | 2703 |
| | 379 | 1623 | 2704 |
| | 380 | 1624 | 2705 |
| | 381 | 1625 | 2706 |
| | 382 | 1626 | 2707 |
| | 383 | 1627 | 2708 |
| | 384 | 1628 | 2709 |
| | 385 | 1629 | 2710 |
| | 386 | 1630 | 2711 |
| | 387 | 163 | 2712 |
| | 388 | 1632 | 2713 |
| | 389 | 1633 | 2714 |
| | 390 | 1634 | 2715 |
| | 391 | 1635 | 2716 |
| | 392 | 1636 | 2717 |
| | 393 | 1637 | 2718 |
| | 394 | 1638 | 2719 |
| | 395 | 1639 | 2720 |
| | 396 | 1640 | 2721 |
| | 397 | 1641 | 2722 |
| | 398 | 1642 | 2723 |
| | 399 | 1643 | 2724 |
| | 400 | 1644 | 2725 |
| | 401 | 1645 | 2726 |
| | 402 | 1646 | 2727 |
| | 403 | 1647 | 2728 |
| | 404 | 1648 | 2729 |
| | 405 | 1649 | 2730 |
| | 406 | 1650 | 2731 |
| | 407 | 1651 | 2732 |
| | 408 | 1652 | 2733 |
| | 409 | 1653 | 2734 |
| | 410 | 1654 | 2735 |
| | 411 | 1655 | 2736 |
| | 412 | 1656 | 2737 |
| | 413 | 1657 | 2738 |
| | 414 | 1658 | 2739 |
| | 415 | 1659 | 2740 |
| | 416 | 1660 | 2741 |
| | 417 | 1661 | 2742 |
| | 418 | 1662 | 2743 |
| | 419 | 1663 | 2744 |
| | 420 | 1664 | 2745 |
| | 421 | 1665 | 2746 |
| | 422 | 1666 | 2747 |
| | 423 | 1667 | 2748 |
| | 424 | 1668 | 2749 |
| | 425 | 1669 | 2750 |
| | 426 | 1670 | 2751 |
| | 427 | 1671 | 2752 |
| | 428 | 1672 | 2753 |
| | 429 | 1673 | 2754 |
| | 430 | 1674 | 2755 |
| | 431 | 1675 | 2756 |
| | 432 | 1676 | 2757 |
| | 433 | 1677 | 2758 |
| | 434 | 1678 | 2759 |
| | 435 | 1679 | 2760 |
| | 436 | 1680 | 2761 |
| | 437 | 1681 | 2762 |
| | 438 | 1682 | 2763 |
| | 439 | 1683 | 2764 |
| | 440 | 1684 | 2765 |
| | 441 | 1685 | 2766 |
| | 442 | 1686 | 2767 |
| | 443 | 1687 | 2768 |
| | 444 | 1688 | 2769 |
| | 445 | 1689 | 2770 |
| | 446 | 1690 | 2771 |
| | 447 | 1691 | 2772 |
| | 448 | 1692 | 2773 |
| | 449 | 1693 | 2774 |
| | 450 | 1694 | 2775 |
| | 451 | 1695 | 2776 |
| | 452 | 1696 | 2777 |
| | 453 | 1697 | 2778 |
| | 454 | 1698 | 2779 |
| | 455 | 1699 | 2780 |
| | 456 | 1700 | 2781 |
| | 457 | 1701 | 2782 |
| | 458 | 1702 | 2783 |
| | 459 | 1703 | 2784 |
| | 460 | 1704 | 2785 |
| | 461 | 1705 | 2786 |
| | 462 | 1706 | 2787 |
| | 463 | 1707 | 2788 |
| | 464 | 1708 | 2789 |
| | 465 | 1709 | 2790 |
| | 466 | 1710 | 2791 |
| | 467 | 1711 | 2792 |
| | 468 | 1712 | 2793 |
| | 469 | 1713 | 2794 |
| | 470 | 1714 | 2795 |
| | 471 | 1715 | 2796 |
| | 472 | 1716 | 2797 |
| | 473 | 1717 | 2798 |
| | 474 | 1718 | 2799 |
| | 475 | 1719 | 2800 |
| | 476 | 1720 | 2801 |
| | 477 | 1721 | 2802 |
| | 478 | 1722 | 2803 |
| | 479 | 1723 | 2804 |
| | 480 | 1724 | 2805 |
| | 481 | 1725 | 2806 |
| | 482 | 1726 | 2807 |
| | 483 | 1727 | 2808 |
| | 484 | 1728 | 2809 |
| | 485 | 1729 | 2810 |
| | 486 | 1730 | 2811 |
| | 487 | 1731 | 2812 |
| | 488 | 1732 | 2813 |
| | 489 | 1733 | 2814 |
| | 490 | 1734 | 2815 |
| | 491 | 1735 | 2816 |
| | 492 | 1736 | 2817 |
| | 493 | 1737 | 2818 |
| | 494 | 1738 | 2819 |
| | 495 | 1739 | 2820 |
| | 496 | 1740 | 2821 |
| | 497 | 1741 | 2822 |
| | 498 | 1742 | 2823 |
| | 499 | 1743 | 2824 |
| | 500 | 1744 | 2825 |
| | 501 | 1745 | 2826 |
| | 502 | 1746 | 2827 |
| | 503 | 1747 | 2828 |
| | 504 | 1748 | 2829 |
| | 505 | 1749 | 2830 |
| | 506 | 1750 | 2831 |
| | 507 | 1751 | 2832 |
| | 508 | 1752 | 2833 |
| | 509 | 1753 | 2834 |
| | 510 | 1754 | 2835 |
| | 511 | 1755 | 2836 |
| | 512 | 1756 | 2837 |
| | 513 | 1757 | 2838 |
| | 514 | 1758 | 2839 |
| | 515 | 1759 | 2840 |
| | 516 | 1760 | 2841 |
| | 517 | 1761 | 2842 |
| | 518 | 1762 | 2843 |
| | 519 | 1763 | 2844 |
| | 520 | 1764 | 2845 |
| | 521 | 1765 | 2846 |
| | 522 | 1766 | 2847 |
| | 523 | 1767 | 2848 |
| | 524 | 1768 | 2849 |
| | 525 | 1769 | 2850 |
| | 526 | 1770 | 2851 |
| | 527 | 1771 | 2852 |
| | 528 | 1772 | 2853 |
| | 529 | 1773 | 2854 |
| | 530 | 1774 | 2855 |
| | 531 | 1775 | 2856 |
| | 532 | 1776 | 2857 |
| | 533 | 1777 | 2858 |
| | 534 | 1778 | 2859 |
| | 535 | 1779 | 2860 |
| | 536 | 1780 | 2861 |
| | 537 | 1781 | 2862 |
| | 538 | 1782 | 2863 |
| | 539 | 1783 | 2864 |
| | 540 | 1784 | 2865 |
| | 541 | 1785 | 2866 |
| | 542 | 1786 | 2867 |
| | 543 | 1787 | 2868 |
| | 544 | 1788 | 2869 |
| | 545 | 1789 | 2870 |
| | 546 | 1790 | 2871 |
| | 547 | 1791 | 2872 |
| | 548 | 1792 | 2873 |
| | 549 | 1793 | 2874 |
| | 550 | 1794 | 2875 |
| | 551 | 1795 | 2876 |
| | 552 | 1796 | 2877 |
| | 553 | 1797 | 2878 |
| | 554 | 1798 | 2879 |
| | 555 | 1799 | 2880 |
| | 556 | 1800 | 2881 |
| | 557 | 1801 | 2882 |
| | 558 | 1802 | 2883 |
| | 559 | 1803 | 2884 |
| | 560 | 1804 | 2885 |
| | 561 | 1805 | 2886 |
| | 562 | 1806 | 2887 |
| | 563 | 1807 | 2888 |
| | 564 | 1808 | 2889 |
| | 565 | 1809 | 2890 |
| | 566 | 1810 | 2891 |
| | 567 | 1811 | 2892 |
| | 568 | 1812 | 2893 |
| | 569 | 1813 | 2894 |
| | 570 | 1814 | 2895 |
| | 571 | 1815 | 2896 |
| | 572 | 1816 | 2897 |
| | 573 | 1817 | 2898 |
| | 574 | 1818 | 2899 |
| | 575 | 1819 | 2900 |
| | 576 | 1820 | 2901 |
| | 577 | 1821 | 2902 |
| | 578 | 1822 | 2903 |
| | 579 | 1823 | 2904 |
| | 580 | 1824 | 2905 |
| | 581 | 1825 | 2906 |
| | 582 | 1826 | 2907 |
| | 583 | 1827 | 2908 |
| | 584 | 1828 | 2909 |
| | 585 | 1829 | 2910 |
| | 586 | 1830 | 2911 |
| | 587 | 1831 | 2912 |
| | 588 | 1832 | 2913 |
| | 589 | 1833 | 2914 |
| | 590 | 1834 | 2915 |
| | 591 | 1835 | 2916 |
| | 592 | 1836 | 2917 |
| | 593 | 1837 | 2918 |
| | 594 | 1838 | 2919 |
| | 595 | 1839 | 2920 |
| | 596 | 1840 | 2921 |
| | 597 | 1841 | 2922 |
| | 598 | 1842 | 2923 |
| | 599 | 1843 | 2924 |
| | 600 | 1844 | 2925 |
| | 601 | 1845 | 2926 |
| | 602 | 1846 | 2927 |
| | 603 | 1847 | 2928 |
| | 604 | 1848 | 2929 |
| | 605 | 1849 | 2930 |
| | 606 | 1850 | 2931 |
| | 607 | 1851 | 2932 |
| | 608 | 1852 | 2933 |
| | 609 | 1853 | 2934 |
| | 610 | 1854 | 2935 |
| | 611 | 1855 | 2936 |
| | 612 | 1856 | 2937 |
| | 613 | 1857 | 2938 |
| | 614 | 1858 | 2939 |
| | 615 | 1859 | 2940 |
| | 616 | 1860 | 2941 |
| | 617 | 1861 | 2942 |
| | 618 | 1862 | 2943 |
| | 619 | 1863 | 2944 |
| | 620 | 1864 | 2945 |
| | 621 | 1865 | 2946 |
| | 622 | 1866 | 2947 |
| | 623 | 1867 | 2948 |
| | 624 | 1868 | 2949 |
| | 625 | 1869 | 2950 |
| | 626 | 1870 | 2951 |
| | 627 | 1871 | 2952 |
| | 628 | 1872 | 2953 |
| | 629 | 1873 | 2954 |
| | 630 | 1874 | 2955 |
| | 631 | 1875 | 2956 |
| | 632 | 1876 | 2957 |
| | 633 | 1877 | 2958 |
| | 634 | 1878 | 2959 |
| | 635 | 1879 | 2960 |
| | 636 | 1880 | 2961 |
| | 637 | 1881 | 2962 |
| | 638 | 1882 | 2963 |
| | 639 | 1883 | 2964 |
| | 640 | 1884 | 2965 |
| | 641 | 1885 | 2966 |
| | 642 | 1886 | 2967 |
| | 643 | 1887 | 2968 |
| | 644 | 1888 | 2969 |
| | 645 | 1889 | 2970 |
| | 646 | 1890 | 2971 |
| | 647 | 1891 | 2972 |
| | 648 | 1892 | 2973 |
| | 649 | 1893 | 2974 |
| | 650 | 1894 | 2975 |
| | 651 | 1895 | 2976 |
| | 652 | 1896 | 2977 |
| | 653 | 1897 | 2978 |
| | 654 | 1898 | 2979 |
| | 655 | 1899 | 2980 |
| | 656 | 1900 | 2981 |
| | 657 | 1901 | 2982 |
| | 658 | 1902 | 2983 |
| | 659 | 1903 | 2984 |
| | 660 | 1904 | 2985 |
| | 661 | 1905 | 2986 |
| | 662 | 1906 | 2987 |
| | 663 | 1907 | 2988 |
| | 664 | 1908 | 2989 |
| | 665 | 1909 | 2990 |
| | 666 | 1910 | 2991 |
| | 667 | 1911 | 2992 |
| | 668 | 1912 | 2993 |
| | 669 | 1913 | 2994 |
| | 670 | 1914 | 2995 |
| | 671 | 1915 | 2996 |
| | 672 | 1916 | 2997 |
| | 673 | 1917 | 2998 |
| | 674 | 1918 | 2999 |
| | 675 | 1919 | 3000 |
| | 676 | 1920 | 3001 |
| | 677 | 1921 | 3002 |
| | 678 | 1922 | 3003 |
| | 679 | 1923 | 3004 |
| | 680 | 1924 | 3005 |
| | 681 | 1925 | 3006 |
| | 682 | 1926 | 3007 |
| | 683 | 1927 | 3008 |
| | 684 | 1928 | 3009 |
| | 685 | 1929 | 3010 |
| | 686 | 1930 | 3011 |
| | 687 | 1931 | 3012 |
| | 688 | 1932 | 3013 |
| | 689 | 1933 | 3014 |
| | 690 | 1934 | 3015 |
| | 691 | 1935 | 3016 |
| | 692 | 1936 | 3017 |
| | 693 | 1937 | 3018 |
| | 694 | 1938 | 3019 |
| | 695 | 1939 | 3020 |
| | 696 | 1940 | 3021 |
| | 697 | 1941 | 3022 |
| | 698 | 1942 | 3023 |
| | 699 | 1943 | 3024 |
| | 700 | 1944 | 3025 |
| | 701 | 1945 | 3026 |
| | 702 | 1946 | 3027 |
| | 703 | 1947 | 3028 |
| | 704 | 1948 | 3029 |
| | 705 | 1949 | 3030 |
| | 706 | 1950 | 3031 |
| | 707 | 1951 | 3032 |
| | 708 | 1952 | 3033 |
| | 709 | 1953 | 3034 |
| | 710 | 1954 | 3035 |
| | 711 | 1955 | 3036 |
| | 712 | 1956 | 3037 |
| | 713 | 1957 | 3038 |
| | 714 | 1958 | 3039 |
| | 715 | 1959 | 3040 |
| | 716 | 1960 | 3041 |
| | 717 | 1961 | 3042 |
| | 718 | 1962 | 3043 |
| | 719 | 1963 | 3044 |
| | 720 | 1964 | 3045 |
| | 721 | 1965 | 3046 |
| | 722 | 1966 | 3047 |
| | 723 | 1967 | 3048 |
| | 724 | 1968 | 3049 |
| | 725 | 1969 | 3050 |
| | 726 | 1970 | 3051 |
| | 727 | 1971 | 3052 |
| | 728 | 1972 | 3053 |
| | 729 | 1973 | 3054 |
| | 730 | 1974 | 3055 |
| | 731 | 1975 | 3056 |
| | 732 | 1976 | 3057 |
| | 733 | 1977 | 3058 |
| | 734 | 1978 | 3059 |
| | 735 | 1979 | 3060 |
| | 736 | 1980 | 3061 |
| | 737 | 1981 | 3062 |
| | 738 | 1982 | 3063 |
| | 739 | 1983 | 3064 |
| | 740 | 1984 | 3065 |
| | 741 | 1985 | 3066 |
| | 742 | 1986 | 3067 |
| | 743 | 1987 | 3068 |
| | 744 | 1988 | 3069 |
| | 745 | 1989 | 3070 |
| | 746 | 1990 | 3071 |
| | 747 | 1991 | 3072 |
| | 748 | 1992 | 3073 |
| | 749 | 1993 | 3074 |
| | 750 | 1994 | 3075 |
| | 751 | 1995 | 3076 |
| | 752 | 1996 | 3077 |
| | 753 | 1997 | 3078 |
| | 754 | 1998 | 3079 |
| | 755 | 1999 | 3080 |
| | 756 | 2000 | 3081 |
| | 757 | 2001 | 3082 |
| | 758 | 2002 | 3083 |
| | 759 | 2003 | 3084 |
| | 760 | 2004 | 3085 |
| | 761 | 2005 | 3086 |
| | 762 | 2006 | 3087 |
| | 763 | 2007 | 3088 |
| | 764 | 2008 | 3089 |
| | 765 | 2009 | 3090 |
| | 766 | 2010 | 3091 |
| | 767 | 2011 | 3092 |
| | 768 | 2012 | 3093 |
| | 769 | 2013 | 3094 |
| | 770 | 2014 | 3095 |
| | 771 | 2015 | 3096 |
| | 772 | 2016 | 3097 |
| | 773 | 2017 | 3098 |
| | 774 | 2018 | 3099 |
| | 775 | 2019 | 3100 |
| | 776 | 2020 | 3101 |
| | 777 | 2021 | 3102 |
| | 778 | 2022 | 3103 |
| | 779 | 2023 | 3104 |
| | 780 | 2024 | 3105 |
| | 781 | 2025 | 3106 |
| | 782 | 2026 | 3107 |
| | 783 | 2027 | 3108 |
| | 784 | 2028 | 3109 |
| | 785 | 2029 | 3110 |
| | 786 | 2030 | 3111 |
| | 787 | 2031 | 3112 |
| | 788 | 2032 | 3113 |
| | 789 | 2033 | 3114 |
| | 790 | 2034 | 3115 |
| | 791 | 2035 | 3116 |
| | 792 | 2036 | 3117 |
| | 793 | 2037 | 3118 |
| | 794 | 2038 | 3119 |
| | 795 | 2039 | 3120 |
| | 796 | 2040 | 3121 |
| | 797 | 2041 | 3122 |
| | 798 | 2042 | 3123 |
| | 799 | 2043 | 3124 |
| | 800 | 2044 | 3125 |
| | 801 | 2045 | 3126 |
| | 802 | 2046 | 3127 |
| | 803 | 2047 | 3128 |
| | 804 | 2048 | 3129 |
| | 805 | 2049 | 3130 |
| | 806 | 2050 | 3131 |
| | 807 | 2051 | 3132 |
| | 808 | 2052 | 3133 |
| | 809 | 2053 | 3134 |
| | 810 | 2054 | 3135 |
| | 811 | 2055 | 3136 |
| | 812 | 2056 | 3137 |
| | 813 | 2057 | 3138 |
| | 814 | 2058 | 3139 |
| | 815 | 2059 | 3140 |
| | 816 | 2060 | 3141 |
| | 817 | 2061 | 3142 |
| | 818 | 2062 | 3143 |
| | 819 | 2063 | 3144 |
| | 820 | 2064 | 3145 |
| | 821 | 2065 | 3146 |
| | 822 | 2066 | 3147 |
| | 823 | 2067 | 3148 |
| | 824 | 2068 | 3149 |
| | 825 | 2069 | 3150 |
| | 826 | 2070 | 3151 |
| | 827 | 2071 | 3152 |
| | 828 | 2072 | 3153 |
| | 829 | 2073 | 3154 |
| | 830 | 2074 | 3155 |
| | 831 | 2075 | 3156 |
| | 832 | 2076 | 3157 |
| | 833 | 2077 | 3158 |
| | 834 | 2078 | 3159 |
| | 835 | 2079 | 3160 |
| | 836 | 2080 | 3161 |
| | 837 | 2081 | 3162 |
| | 838 | 2082 | 3163 |
| | 839 | 2083 | 3164 |
| | 840 | 2084 | 3165 |
| | 841 | 2085 | 3166 |
| | 842 | 2086 | 3167 |
| | 843 | 2087 | 3168 |
| | 844 | 2088 | 3169 |
| | 845 | 2089 | 3170 |
| | 846 | 2090 | 3171 |
| | 847 | 2091 | 3172 |
| | 848 | 2092 | 3173 |
| | 849 | 2093 | 3174 |
| | 850 | 2094 | 3175 |
| | 851 | 2095 | 3176 |
| | 852 | 2096 | 3177 |
| | 853 | 2097 | 3178 |
| | 854 | 2098 | 3179 |
| | 855 | 2099 | 3180 |
| | 856 | 21.00 | 3181 |
| | 857 | 2101 | 3182 |
| | 858 | 2102 | 3183 |
| | 859 | 2103 | 3184 |
| | 860 | 2104 | 3185 |
| | 861 | 2105 | 3186 |
| | 862 | 2106 | 3187 |
| | 863 | 2107 | 3188 |
| | 864 | 2108 | 3189 |
| | 865 | 2109 | 3190 |
| | 866 | 2110 | 3191 |
| | 867 | 2111 | 3192 |
| | 868 | 2112 | 3193 |
| | 869 | 2113 | 3194 |
| | 870 | 2114 | 3195 |
| | 871 | 2115 | 3196 |
| | 872 | 2116 | 3197 |
| | 873 | 2117 | 3198 |
| | 874 | 2118 | 3199 |
| | 875 | 2119 | 3200 |
| | 876 | 2120 | 3201 |
| | 877 | 2121 | 3202 |
| | 878 | 2122 | 3203 |
| | 879 | 2123 | 3204 |
| | 880 | 2124 | 3205 |
| | 881 | 2125 | 3206 |
| | 882 | 2126 | 3207 |
| | 883 | 2127 | 3208 |
| | 884 | 2128 | 3209 |
| | 885 | 2129 | 3210 |
| | 886 | 2130 | 3211 |
| | 887 | 2131 | 3212 |
| | 888 | 2132 | 3213 |
| | 889 | 2133 | 3214 |
| | 890 | 2134 | 3215 |
| | 891 | 2135 | 3216 |
| | 892 | 2136 | 3217 |
| | 893 | 2137 | 3218 |
| | 894 | 2138 | 3219 |
| | 895 | 2139 | 3220 |
| | 896 | 2140 | 3221 |
| | 897 | 2141 | 3222 |
| | 898 | 2142 | 3223 |
| | 899 | 2143 | 3224 |
| | 900 | 2144 | 3225 |
| | 901 | 2145 | 3226 |
| | 902 | 2146 | 3227 |
| | 903 | 2147 | 3228 |
| | 904 | 2148 | 3229 |
| | 905 | 2149 | 3230 |
| | 906 | 2150 | 3231 |
| | 907 | 2151 | 3232 |
| | 908 | 2152 | 3233 |
| | 909 | 2153 | 3234 |
| | 910 | 2154 | 3235 |
| | 911 | 2155 | 3236 |
| | 912 | 2156 | 3237 |
| | 913 | 2157 | 3238 |
| | 914 | 2158 | 3239 |
| | 915 | 2159 | 3240 |
| | 916 | 2160 | 3241 |
| | 917 | 2161 | 3242 |
| | 918 | 2162 | 3243 |
| | 919 | 2163 | 3244 |
| | 920 | 2164 | 3245 |
| | 921 | 2165 | 3246 |
| | 922 | 2166 | 3247 |
| | 923 | 2167 | 3248 |
| | 924 | 2168 | 3249 |
| | 925 | 2169 | 3250 |
| | 926 | 2170 | 3251 |
| | 927 | 2171 | 3252 |
| | 928 | 2172 | 3253 |
| | 929 | 2173 | 3254 |
| | 930 | 2174 | 3255 |
| | 931 | 2175 | 3256 |
| | 932 | 2176 | 3257 |
| | 933 | 2177 | 3258 |
| | 934 | 2178 | 3259 |
| | 935 | 2179 | 3260 |
| | 936 | 2180 | 3261 |
| | 937 | 2181 | 3262 |
| | 938 | 2182 | 3263 |
| | 939 | 2183 | 3264 |
| | 940 | 2184 | 3265 |
| | 941 | 2185 | 3266 |
| | 942 | 2186 | 3267 |
| | 943 | 2187 | 3268 |
| | 944 | 2188 | 3269 |
| | 945 | 2189 | 3270 |
| | 946 | 2190 | 3271 |
| | 947 | 2191 | 3272 |
| | 948 | 2192 | 3273 |
| | 949 | 2193 | 3274 |
| | 950 | 2194 | 3275 |
| | 951 | 2195 | 3276 |
| | 952 | 2196 | 3277 |
| | 953 | 2197 | 3278 |
| | 954 | 2198 | 3279 |
| | 955 | 2199 | 3280 |
| | 956 | 2200 | 3281 |
| | 957 | 2201 | 3282 |
| | 958 | 2202 | 3283 |
| | 959 | 2203 | 3284 |
| | 960 | 2204 | 3285 |
| | 961 | 2205 | 3286 |
| | 962 | 2206 | 3287 |
| | 963 | 2207 | 3288 |
| | 964 | 2208 | 3289 |
| | 965 | 2209 | 3290 |
| | 966 | 2210 | 3291 |
| | 967 | 2211 | 3292 |
| | 968 | 2212 | 3293 |
| | 969 | 2213 | 3294 |
| | 970 | 2214 | 3295 |
| | 971 | 2215 | 3296 |
| | 972 | 2216 | 3297 |
| | 973 | 2217 | 3298 |
| | 974 | 2218 | 3299 |
| | 975 | 2219 | 3300 |
| | 976 | 2220 | 3301 |
| | 977 | 2221 | 3302 |
| | 978 | 2222 | 3303 |
| | 979 | 2223 | 3304 |
| | 980 | 2224 | 3305 |
| | 981 | 2225 | 3306 |
| | 982 | 2226 | 3307 |
| | 983 | 2227 | 3308 |
| | 984 | 2228 | 3309 |
| | 985 | 2229 | 3310 |
| | 986 | 2230 | 3311 |
| | 987 | 2231 | 3312 |
| | 988 | 2232 | 3313 |
| | 989 | 2233 | 3314 |
| | 990 | 2234 | 3315 |
| | 991 | 2235 | 3316 |
| | 992 | 2236 | 3317 |
| | 993 | 2237 | 3318 |
| | 994 | 2238 | 3319 |
| | 995 | 2239 | 3320 |
| | 996 | 2240 | 3321 |
| | 997 | 2241 | 3322 |
| | 998 | 2242 | 3323 |
| | 999 | 2243 | 3324 |
| | 1000 | 2244 | 3325 |
| | 1001 | 2245 | 3326 |
| | 1002 | 2246 | 3327 |
| | 1003 | 2247 | 3328 |
| | 1004 | 2248 | 3329 |
| | 1005 | 2249 | 3330 |
| | 1006 | 2250 | 3331 |
| | 1007 | 2251 | 3332 |
| | 1008 | 2252 | 3333 |
| | 1009 | 2253 | 3334 |
| | 1010 | 2254 | 3335 |
| | 1011 | 2255 | 3336 |
| | 1012 | 2256 | 3337 |
| | 1013 | 2257 | 3338 |
| | 1014 | 2258 | 3339 |
| | 1015 | 2259 | 3340 |
| | 1016 | 2260 | 3341 |
| | 1017 | 2261 | 3342 |
| | 1018 | 2262 | 3343 |
| | 1019 | 2263 | 3344 |
| | 1020 | 2264 | 3345 |
| | 1021 | 2265 | 3346 |
| | 1022 | 2266 | 3347 |
| | 1023 | 2267 | 3348 |
| | 1024 | 2268 | 3349 |
| | 1025 | 2269 | 3350 |
| | 1026 | 2270 | 3351 |
| | 1027 | 2271 | 3352 |
| | 1028 | 2272 | 3353 |
| | 1029 | 2273 | 3354 |
| | 1030 | 2274 | 3355 |
| | 1031 | 2275 | 3356 |
| | 1032 | 2276 | 3357 |
| | 1033 | 2277 | 3358 |
| | 1034 | 2278 | 3359 |
| | 1035 | 2279 | 3360 |
| | 1036 | 2280 | 3361 |
| | 1037 | 2281 | 3362 |
| | 1038 | 2282 | 3363 |
| | 1039 | 2283 | 3364 |
| | 1040 | 2284 | 3365 |
| | 1041 | 2285 | 3366 |
| | 1042 | 2286 | 3367 |
| | 1043 | 2287 | 3368 |
| | 1044 | 2288 | 3369 |
| | 1045 | 2289 | 3370 |
| | 1046 | 2290 | 3371 |
| | 1047 | 2291 | 3372 |
| | 1048 | 2292 | 3373 |
| | 1049 | 2293 | 3374 |
| | 1050 | 2294 | 3375 |
| | 1051 | 2295 | 3376 |
| | 1052 | 2296 | 3377 |
| | 1053 | 2297 | 3378 |
| | 1054 | 2298 | 3379 |
| | 1055 | 2299 | 3380 |
| | 1056 | 2300 | 3381 |
| | 1057 | 2301 | 3382 |
| | 1058 | 2302 | 3383 |
| | 1059 | 2303 | 3384 |
| | 1060 | 2304 | 3385 |
| | 1061 | 2305 | 3386 |
| | 1062 | 2306 | 3387 |
| | 1063 | 2307 | 3388 |
| | 1064 | 2308 | 3389 |
| | 1065 | 2309 | 3390 |
| | 1066 | 2310 | 3391 |
| | 1067 | 2311 | 3392 |
| | 1068 | 2312 | 3393 |
| | 1069 | 2313 | 3394 |
| | 1070 | 2314 | 3395 |
| | 1071 | 2315 | 3396 |
| | 1072 | 2316 | 3397 |
| | 1073 | 2317 | 3398 |
| | 1074 | 2318 | 3399 |
| | 1075 | 2319 | 3400 |
| | 1076 | 2320 | 3401 |
| | 1077 | 2321 | 3402 |
| | 1078 | 2322 | 3403 |
| | 1079 | 2323 | 3404 |
| | 1080 | 2324 | 3405 |
| | 1081 | 2325 | 3406 |
| | 1082 | 2326 | 3407 |
| | 1083 | 2327 | 3408 |
| | 1084 | 2328 | 3409 |
| | 1085 | 2329 | 3410 |
| | 1086 | 2330 | 3411 |
| | 1087 | 2331 | 3412 |
| | 1088 | 2332 | 3413 |
| | 1089 | 2333 | 3414 |
| | 1090 | 2334 | 3415 |
| | 1091 | 2335 | 3416 |
| | 1092 | 2336 | 3417 |
| | 1093 | 2337 | 3418 |
| | 1094 | 2338 | 3419 |
| | 1095 | 2339 | 3420 |
| | 1096 | 2340 | 3421 |
| | 1097 | 2341 | 3422 |
| | 1098 | 2342 | 3423 |
| | 1099 | 2343 | 3424 |
| | 1100 | 2344 | 3425 |
| | 1101 | 2345 | 3426 |
| | 1102 | 2346 | 3427 |
| | 1103 | 2347 | 3428 |
| | 1104 | 2348 | 3429 |
| | 1105 | 2349 | 3430 |
| | 1106 | 2350 | 3431 |
| | 1107 | 2351 | 3432 |
| | 1108 | 2352 | 3433 |
| | 1109 | 2353 | 3434 |
| | 1110 | 2354 | 3435 |
| | 1111 | 2355 | 3436 |
| | 1112 | 2356 | 3437 |
| | 1113 | 2357 | 3438 |
| | 1114 | 2358 | 3439 |
| | 1115 | 2359 | 3440 |
| | 1116 | 2360 | 3441 |
| | 1117 | 2361 | 3442 |
| | 1118 | 2362 | 3443 |
| | 1119 | 2363 | 3444 |
| | 1120 | 2364 | 3445 |
| | 1121 | 2365 | 3446 |
| | 1122 | 2366 | 3447 |
| | 1123 | 2367 | 3448 |
| | 1124 | 2368 | 3449 |
| | 1125 | 2369 | 3450 |
| | 1126 | 2370 | 3451 |
| | 1127 | 2371 | 3452 |
| | 1128 | 2372 | 3453 |
| | 1129 | 2373 | 3454 |
| | 1130 | 2374 | 3455 |
| | 1131 | 2375 | 3456 |
| | 1132 | 2376 | 3457 |
| | 1133 | 2377 | 3458 |
| | 1134 | 2378 | 3459 |
| | 1135 | 2379 | 3460 |
| | 1136 | 2380 | 3461 |
| | 1137 | 2381 | 3462 |
| | 1138 | 2382 | 3463 |
| | 1139 | 2383 | 3464 |
| | 1140 | 2384 | 3465 |
| | 1141 | 2385 | 3466 |
| | 1142 | 2386 | 3467 |
| | 1143 | 2387 | 3468 |
| | 1144 | 2388 | 3469 |
| | 1145 | 2389 | 3470 |
| | 1146 | 2390 | 3471 |
| | 1147 | 2391 | 3472 |
| | 1148 | 2392 | 3473 |
| | 1149 | 2393 | 3474 |
| | 1150 | 2394 | 3475 |
| | 1151 | 2395 | 3476 |
| | 1152 | 2396 | 3477 |
| | 1153 | 2397 | 3478 |
| | 1154 | 2398 | 3479 |
| | 1155 | 2399 | 3480 |
| | 1156 | 2400 | 3481 |
| | 1157 | 2401 | 3482 |
| | 1158 | 2402 | 3483 |
| | 1159 | 2403 | 3484 |
| | 1160 | 2404 | 3485 |
| | 1161 | 2405 | 3486 |
| | 1162 | 2406 | 3487 |
| | 1163 | 2407 | 3488 |
| | 1164 | 2408 | 3489 |
| | 1165 | 2409 | 3490 |
| | 1166 | 2410 | 3491 |
| | 1167 | 2411 | 3492 |
| | 1168 | 2412 | 3493 |

Table 5:
LSR Protein
sequence
SEQ ID
NOs:
1169
1170
1171
1172
1173
1174
1175
1176
1177
1178
1179
1180
1181
1182
1183

What is claimed is:

1. A system for DNA modification comprising:
a polypeptide comprising a recombinase having an amino acid sequence having at least 70% identity to any of SEQ ID NOs: 1-74, or a nucleic acid encoding thereof; and
a first polynucleotide comprising a donor recognition sequence for the recombinase.

2. The system of claim 1, wherein the recombinase has an amino acid sequence having at least 70% identity to any of SEQ ID NOs: 2, 6, 10, 12, 18, 19, 26, 29, 61, 65, or 66.

3. A system for DNA modification comprising:
a polypeptide comprising a recombinase having an amino acid sequence with at least 70% identity to one or more of the following:
1) X₁ₐX₂ₐX₃ₐX₄ₐX₅ₐX₆ₐX₇ₐX₈ₐX₉ₐX₁₀ₐX₁₁ₐX₁₂ₐX₁₃ₐX₁₄ₐX₁₅ₐX₁₆ₐX₁₇ₐX₁₈ₐX₁₉ₐX₂₀ₐX₂₁ₐX₂₂ₐX₂₃ₐX₂₄ₐX₂₅ₐX₂₆ₐX₂₇ₐX₂₈ₐX₂₉ₐX₃₀ₐX₃₁ₐX₃₂ₐX₃₃ₐX₃₄ₐ, wherein:
X₁ₐ is A, E, I, L, S, T, V, or Y;
X₂ₐ is A, D, E, G, K, Q, R, S, or T;
X₆ₐ is E or G;
X₈ₐ is A, C, F, L, M, or V;
X₁₀ₐ is A, F, I, L, M, T, or V;
X₁₃ₐ is F, H, I, L, M, N, or V;
X₁₄ₐ is A, G, S, or V;
X₁₅ₐ is A, D, I, L, S, T, or V;
X₁₇ₐ is A, G, or S;
X₂₁ₐ is K, R, S, or V;
X₂₂ₐ is A, D, E, G, K, N, S, or T;
X₂₃ₐ is A, E, I, K, M, N, Q, S, or T;
X₂₄ₐ is F, I, L, M, S, or T;
X₂₆ₐ is D, E, L, Q, S, or V;
X₂₇ₐ is E, N, Q, or R;
X₃₂ₐ is A, F, H, I, K, L, M, N, Q, R, S, or V
X₃₄ₐ is A, E, G, H, K, L, M, N, Q, R, S, or V; and
X₃ₐ, X₄ₐ, X₅ₐ, X₇ₐ, X₉ₐ, X₁₁ₐ, X₁₂ₐ, X₁₆ₐ, X₁₈ₐ, X₁₉ₐ, X₂₀ₐ, X₂₅ₐ, X₂₈ₐ, X₂₉ₐ, X₃₀ₐ, X₃₁ₐ, and X₃₃ₐ are each individually selected from any amino acid;
2) X_{1b}X_{2b}X_{3b}X_{4b}X_{5b}X_{6b}X_{7b}X_{8b}X_{9b}X_{10b}X_{11b}X_{12b}X_{13b}X_{14b}X_{15b}X_{16b}X_{17b}X_{18b}, wherein
X_{1b} is A, G, or I;
X_{2b} is D, E, G, N, P, S, T, or V;
X_{3b} is D, G, N, Q, or S;
X_{4b} is A, H, N, Q, R, T, V, or Y;
X_{6b} is A, D, E, H, I, L, P, Q, R, T, or Y;
X_{7b} is A, D, E, Q, or R;
X_{8b} is F, I, K, or L;
X_{10b} is D, E, F, G, N, Q, R, S, T, or V;
X_{11b} is A, I, L, S, T, or V;
X_{12b} is D, E, I, K, L, N, Q, R, S, T, or V;
X_{13b} is A, D, E, K, M, N, R, S, T, or V;
X_{14b} is A, G, Q, R, S, or T;
X_{16b} is A, D, E, K, L, Q, R, or T; and
X_{18b} is A, L, M, or V; and
X_{5b}, X_{9b}, X_{15b}, and X_{17b} are each individually selected from any amino acid;
3) X_{1c}X_{2c}X_{3c}X_{4c}X_{5c}X_{6c}X_{7c}X_{8c}X_{9c}X_{10c}X_{11c}X_{12c}X_{13c}ESX_{16c}X_{17c}KX_{19c}X_{20c}X_{21c}X_{22c}X_{23c}X_{24c}X_{25c}X_{26c}, wherein
X_{1c} is A, D, F, I, L, M, N, S, or Y;
X_{4c} is A, I, K, M, S, or V;
X_{6c} is A, F, G, I, L, M, or V;
X_{10c} is Q, R, or T;
X_{11c} is A, G, or S;
X_{13c} is D, E, G, N, Q, or S;
X_{17c} is A, H, K, N, R, S, T, or V;
X_{21c} is L, M, R, or Y;
X_{22c} is A, I, N, Q, S, T, or V;
X_{23c} is A, E, F, I, K, L, N, R, T, or V;
X_{25c} is A, F, H, L, N, Q, S, T, or Y;
X_{26c} is A, I, L, M, N, R, S, T, V, or Y; and
X_{2c}, X_{3c}, X_{5c}, X_{7c}, X_{8c}, X_{9c}, X_{12c}, X_{16c}, X_{19c}, X_{20c}, and X_{24c} are each individually selected from any amino acid;
4) X_{1d}X_{2d}X_{3d}X_{4d}X_{5d}X_{6d}X_{7d}X_{8d}X_{9d}X_{10d}X_{11d}X_{12d}X_{13d}X_{14d}X_{15d}X_{16d}X_{17d}X_{18d}X_{19d}X_{20d}X_{21d}X_{22d}X_{23d}X_{24d}X_{25d}X_{26d}X_{27d}X_{28d}, wherein:
X_{1d} is E, K, N, T, G, S, L, D, V, A, R, or P;
X_{2d} is E, H, I, T, G, S, L, D, V, A, or P;
X_{4d} is M, I, T, S, L, V, A, R or P;
X_{5d} is E, K, N, I, T, G, S, D, Q, V, A, R, or P;
X_{6d} is E, G, S, D, A, R, or P;
X_{7d} is I, L, D, A, or R;
X_{8d} is M, H, K, T, L, V, Q, D, A, or R;
X_{9d} is E, K, I, T, G, S, L, D, Q, V, or A;
X_{10d} is E, K, H, D, Q, V, A, or R;
X_{11d} is M, H, I, S, L, V, Q, A, or R;
X_{12d} is Q, E, K, N, M, S, L, D, V, A, or R;
X_{13d} is E, K, H, G, S, L, D, Q, A, or R;
X_{14d} is E, Y, K, N, I, H, L, V, or A;
X_{16d} is E, K, I, T, G, S, L, D, Q, A, or R;
X_{17d} is E, K, H, T, G, D, Q, A, or R:
X_{19d} is Q, E, K, N, T, G, S, D, V, A, or R;
X_{20d} is Q, E, K, N, T, G, S, V, D, A, or R;
X_{21d} is I, S, W, L, V, F, A, or R;
X_{22d} is Q, E, M, T, G, S, L, V, D, or A;
X_{23d} is E, K, N, I, T, G, S, D, A, R, or P;
X_{24d} is E, M, I, L, D, Q, or A:
X_{25d} is E, Y, I, L, V, F, A, or R;
X_{26d} is E, M, T, G, S, L, D, V, A, or R;
X_{27d} is E, K, N, G, S, L, D, Q, A, or R;
X_{28d} is Q, E, G, V, D, A, R, or P; and
X_{3d}, X_{15d}, and X_{18d} are each individually selected from any amino acid;
5) X₁ₑX₂ₑX₃ₑX₄ₑX₅ₑX₆ₑX₇ₑX₈ₑX₉ₑX₁₀ₑX₁₁ₑX₁₂ₑX₁₃ₑX₁₄ₑX₁₅ₑX₁₆ₑX₁₇ₑX₁₈ₑ, wherein:
X₁ₑ is A, D, E, H, K, N, Q, R, or S;
X₂ₑ is A, D, E, F, G, H, K, M, N, Q, R, S, W, or Y;
X₃ₑ is E, F, or Y;
X₄ₑ is F, H, L, W, or Y;
X₆ₑ is A, D, E, F, I, K, L, M, N, Q, R, S, T, or Y;
X₇ₑ is F, I, Q, S, T, or V;
X₈ₑ is A, G, K, L, N, R, S, T, or V;
X₉ₑ is A, D, E, H, K, N, Q, R, T, or Y;
X₁₀ₑ is I, N, Q, or R;
X₁₁ₑ is F, I, L, M, Q, or S;
X₁₄ₑ is A, G, K, N, or S;
X₁₅ₑ is K, M, Q, R, S, T, or V;
X₁₈ₑ is A, E, G, K, M, N, S, T, or Y; and
X₅ₑ, X₁₂ₑ, X₁₃ₑ, X₁₆ₑ, and X₁₇ₑ are each individually selected from any amino acid;
6) WX_{2f}X_{3f}X_{4f}X_{5f}X_{6f}X_{7f}X_{8f}X_{9f}X_{10f}X_{11f}X_{12f}X_{13f}X_{14f}X_{15f}X_{16f}GX_{18f}X_{19f}X_{20f}X_{21f}X_{22f}X_{23f}, wherein:
X_{2f} is A, E, H, N, R, S, T, or V;
X_{4f} is A, G, N, S, or T;
X_{5f} is F, G, L, M, N, Q, S, T, or V;
X_{6f} is I, L, P, or V;
X_{9f} is I, L, T, or V;
X_{14f} is A, C, G, M, Q, R, S, or T;
X_{16f} is I, L, V, or Y;
X_{18f} is D, E, H, N, Q, or S;
X_{20f} is E, H, I, L, M, Q, R, or T;
X_{21f} is A, E, F, H, L, N, P, or Y;
X_{22f} is C, F, H, K, M, N, Q, R, T, or Y;
X_{23f} is D, E, F, I, K, L, N, Q, R, S, T, or V; and
X_{3f}, X_{7f}, X_{8f} X_{10f}, X_{11f}, X_{12f}, X_{13f}, X_{15f}, and X_{19f} are each individually selected from any amino acid;
7) X_{1g}X_{2g}X_{3g}X_{4g}X_{5g}EX_{7g}X_{8g}X_{9g}X_{10g}X_{11g}X_{12g}RX_{14g}X_{15g}X_{16g}X_{17g}X_{18g}X_{19g}X_{20g}X_{21g}, wherein:
X_{1g} is A, G, I, N, S, T, or V;
X_{3g} is A, I, or S;
X_{5g} is F, I, L, M, or Y;
X_{7g} is I or R;
X_{10g} is D, I, L, or T;
X_{12g} is A, E, I, K, M, Q, or S;
X_{14g} is I, T, or V;
X_{16g} is A, D, G, R, S, or T;
X_{18g} is F, K, L, M, or Y;
X_{19g} is A, E, H, I, K, L, M, N, Q, R, V, W, or Y;
X_{21g} is A, I, K, L, M, or R; and
X_{2g}, X_{4g}, X_{8g}, X_{9g}, X_{11g}, X_{15g}, X_{17g}, and X_{20g} are each individually selected from any amino acid;
8) X₁ₕX₂ₕX₃ₕX₄ₕX₅ₕX₆ₕX₇ₕX₈ₕX₉ₕX₁₀ₕX₁₁ₕ, wherein:
X₁ₕ is F or Y;
X₂ₕ is D, E, K, Q, or S;
X₃ₕ is E, K, L, M, or Q;
X₄ₕ is K, L, or R:
X₅ₕ is K, L, or V;
X₇ₕ is G or N;
X₈ₕ is D, E, H, K, L, M, or R;
X₉ₕ is S or T;
X₁₁ₕ is F, H, I, Q, S, T, V, or W; and
X₆ₕ and X₁₀ₕ are each individually selected from any amino acid;
9) X₁ᵢX₂ᵢX₃ᵢX₄ᵢX₅ᵢX₆ᵢX₇ᵢX₈ᵢX₉ᵢX₁₀ᵢX₁₁ᵢX₁₃ᵢX₁₄ᵢX₁₅ᵢX₁₆ᵢX₁₇ᵢX₁₈ᵢX₁₉ᵢX₂₀ᵢX₂₁ᵢX₂₂ᵢX₂₃ᵢX₂₄ᵢX₂₅ᵢX₂₆ᵢX₂₇ᵢ, wherein:
X₁ᵢ is I, L, or V;
X₄ᵢ is A, D, F, H, I, L, M, N, Q, S, V, or Y;
X₈ᵢ is A, G, or S;
X₁₀ᵢ is D, E, I, K, N, Q, R, or S;
X₁₁ᵢ is E or Q;
X₁₅ᵢ is A or K;
X₁₆ᵢ is A, Q, R, or S;
X₁₈ᵢ is L, M, or R;
X₁₉ᵢ is I, L, Q, R, S, or V;
X₂₁ᵢ is A, D, E, G, H, I, Q, R, or S;
X₂₂ᵢ is A, K, N, Q, S, T, or V;
X₂₃ᵢ is A, H, K, R, W, or Y;
X₂₅ᵢ is A, G, H, I, K, Q, R, S, or T;
X₂₇ᵢ is C, H, I, K, L, R, or V; and
X₂ᵢ, X₃ᵢ, X₅ᵢ, X₆ᵢ, X₇ᵢ, X₉ᵢ, X₁₃ᵢ, X₁₄ᵢ, X₁₇ᵢ, X₂₀ᵢ, X₂₄ᵢ, and X₂₆ᵢ are each individually selected from any amino acid;
10) RX₂ⱼX₃ⱼX₄ⱼW, wherein:
X₂ⱼ is L, M, Q, or R;
X₃ⱼ is A, N, or S; and
X₄ⱼ is N, P, S, or T;
11) X₁ₖX₂ₖX₃ₖX₄ₖX₅ₖX₆ₖX₇ₖX₈ₖF, wherein:
X₁ₖ is I, L, or V;
X₂ₖ is A or V;
X₄ₖ is A, F, H, I, L, Q, W, or Y;
X₅ₖ is I, M, or V;
X₇ₖ is E, L, Q, or T:
X₈ₖ is A, I, or V; and
X₃ₖ and X₆ₖ are each individually selected from any amino acid;
12) RX₂ₗX₃ₗX₄ₗX₅ₗX₆ₗX₇ₗX₈ₗX₉ₗX₁₀ₗX₁₁ₗX₁₂ₗX₁₃ₗ, wherein:
X₂ₗ is D, K, N, R, S, or V;
X₃ₗ is A, D, E, F, G, K, P, Q, or S;
X₄ₗ is A, E, I, K, L, S, T, or V;
X₅ₗ is any amino acid:
X₆ₗ is F, G, I, L, N, or V;
X₇ₗ is A, F, I, L, Q, R, V, or Y;
X₈ₗ is D, E, I, L, M, N, Q, S, T, or V;
X₉ₗ is D, E, F, I, L, M, Q, T, V, or Y;
X₁₀ₗ is I, K, L, R, or V;
X₁₁ₗ is D, E, K, N, Q, or R;
X₁₂ₗ is D, E, F, K, L, N, Q, W, or Y; and
X₁₃ₗ is F or L; and
13) X₁ₘX₂ₘX₃ₘX₄ₘX₅ₘX₆ₘX₇ₘX₈ₘX₉ₘX₁₀ₘX₁₁ₘX₁₂ₘX₁₃ₘX₁₄ₘX₁₅ₘX₁₆ₘX₁₇ₘX₁₈ₘX₁₉ₘX₂₀ₘX₂₁ₘX₂₂ₘX₂₃ₘX₂₄ₘ, wherein:
X₁ₘ is A, E, F, I, L, M, N, Q, S, T, V, or Y;
X₂ₘ is A, F, G, I, L, M, R, S, T, or V;
X₆ₘ is A, D, E, F, G, H, L, M, N, S, or T;
X₉ₘ is D, M, N, or S;
X₁₀ₘ is D, E, or Q;
X₁₂ₘ is C, F, H, L, T, V, or Y;
X₁₄ₘ is A, E, K, L, R, or Y;
X₁₇ₘ is A, L, or S;
X₁₉ₘ is D, E, K, N, Q, R, or S;
X₂₀ₘ is G, I, M, Q, R, T, or V;
X₂₁ₘ is D, H, K, N, Q, or R;
X₂₃ₘ is A, G, I, L, N, S, T, or V;
X₂₄ₘ is F, H, I, K, L, M, N, Q, V, W, or Y; and
X₃ₘ, X₄ₘ, X₅ₘ, X₇ₘ, X₈ₘ, X₁₁ₘ, X₁₃ₘ, X₁₅ₘ, X₁₆ₘ, X₁₈ₘ, and X₂₂ₘ, are each individually selected from any amino acid,
or active fragments thereof, or a nucleic acid encoding thereof; and
a first polynucleotide comprising a donor recognition sequence for the recombinase.

4. A system for DNA modification comprising:
a polypeptide comprising a recombinase having an amino acid sequence having at least 70% identity to any of SEQ ID NOs: 88-1183, or a nucleic acid encoding thereof; and
a first polynucleotide comprising a donor recognition sequence for the recombinase.

5. The system of any of claims 1-4, wherein the donor recognition sequence comprises a donor attachment site configured to bind the recombinase.

6. The system of any of claims 1-5, wherein the first polynucleotide further comprises a cargo DNA sequence.

7. The system of claim 6, wherein the cargo DNA sequence is greater than 1 kilobase pair.

8. The system of claim 6 or claim 7, wherein the cargo DNA sequence is greater than 5 kilobase pairs.

9. The system of any of claims 1-8, wherein the first polynucleotide further comprises a recipient recognition sequence for the recombinase.

10. The system of any of claims 1-8, wherein the system further comprises a second polynucleotide comprising a recipient recognition sequence for the recombinase.

11. The system of claim 9 or claim 10, wherein the recipient recognition sequence comprises a recipient attachment sequence configured to bind to the recombinase.

12. The system of any of claims 1-11, wherein the donor recognition sequence, the recipient recognition sequence, or both are pseudo-recognition sequences.

13. The system of any of claims 1-12, wherein the system is a cell free system.

14. A composition comprising the system of any one of claims 1-12.

15. A cell comprising the system of any one of claims 1-12.

16. The cell of claim 15, wherein the cell is a eukaryotic cell.

17. A method of altering a target DNA comprising contacting the target DNA with a polypeptide comprising a recombinase having an amino acid sequence having at least 70% identity to any of SEQ ID NOs: 1-74, or a nucleic acid encoding thereof.

18. A method of altering a target DNA comprising contacting the target DNA with
a polypeptide comprising a recombinase having an amino acid sequence with at least 70% identity to one or more of the following:
1) X₁ₐX₂ₐX₃ₐX₄ₐX₅ₐX₆ₐX₇ₐX₈ₐX₉ₐX₁₀ₐX₁₁ₐX₁₂ₐX₁₃ₐX₁₄ₐX₁₅ₐX₁₆ₐX₁₇ₐX₁₈ₐX₁₉ₐX₂₀ₐX₂₁ₐX₂₂ₐX₂₃ₐX₂₄ₐX₂₅ₐX₂₆ₐX₂₇ₐX₂₈ₐX₂₉ₐX₃₀ₐX₃₁ₐX₃₂ₐX₃₃ₐX₃₄ₐ, wherein:
X₁ₐ is A, E, I, L, S, T, V, or Y;
X₂ₐ is A, D, E, G, K, Q, R, S, or T;
X₆ₐ is E or G;
X₈ₐ is A, C, F, L, M, or V;
X₁₀ₐ is A, F, I, L, M, T, or V;
X₁₃ₐ is F, H, I, L, M, N, or V;
X₁₄ₐ is A, G, S, or V;
X₁₅ₐ is A, D, I, L, S, T, or V;
X₁₇ₐ is A, G, or S;
X₂₁ₐ is K, R, S, or V;
X₂₂ₐ is A, D, E, G, K, N, S, or T;
X₂₃ₐ is A, E, I, K, M, N, Q, S, or T;
X₂₄ₐ is F, I, L, M, S, or T;
X₂₆ₐ is D, E, L, Q, S, or V;
X₂₇ₐ is E, N, Q, or R;
X₃₂ₐ is A, F, H, I, K, L, M, N, Q, R, S, or V
X₃₄ₐ is A, E, G, H, K, L, M, N, Q, R, S, or V; and
X₃ₐ, X₄ₐ, X₅ₐ, X₇ₐ, X₉ₐ, X₁₁ₐ, X₁₂ₐ, X₁₆ₐ, X₁₈ₐ, X₁₉ₐ, X₂₀ₐ, X₂₅ₐ, X₂₈ₐ, X₂₉ₐ, X₃₀ₐ, X₃₁ₐ, and X₃₃ₐ are each individually selected from any amino acid;
2) X_{1b}X_{2b}X_{3b}X_{4b}X_{5b}X_{6b}X_{7b}X_{8b}X_{9b}X_{10b}X_{11b}X_{12b}X_{13b}X_{14b}X_{15b}X_{16b}X_{17b}X_{18b}, wherein
X_{1b} is A, G, or I;
X_{2b} is D, E, G, N, P, S, T, or V;
X_{3b} is D, G, N, Q, or S;
X_{4b} is A, H, N, Q, R, T, V, or Y;
X_{6b} is A, D, E, H, I, L, P, Q, R, T, or Y;
X_{7b} is A, D, E, Q, or R;
X_{8b} is F, I, K, or L;
X_{10b} is D, E, F, G, N, Q, R, S, T, or V;
X_{11b} is A, I, L, S, T, or V;
X_{12b} is D, E, I, K, L, N, Q, R, S, T, or V;
X_{13b} is A, D, E, K, M, N, R, S, T, or V;
X_{14b} is A, G, Q, R, S, or T;
X_{16b} is A, D, E, K, L, Q, R, or T; and
X_{18b} is A, L, M, or V; and
X_{5b}, X_{9b}, X_{15b}, and X_{17b} are each individually selected from any amino acid;
3) X_{1c}X_{2c}X_{3c}X_{4c}X_{5c}X_{6c}X_{7c}X_{8c}X_{9c}X_{10c}X_{11c}X_{12c}X_{13c}ESX_{16c}X_{17c}KX_{19c}X_{20c}X_{21c}X_{22c}X_{23c}X_{24c}X_{25c}X_{26c}, wherein
X_{1c} is A, D, F, I, L, M, N, S, or Y;
X_{4c} is A, I, K, M, S, or V;
X_{6c} is A, F, G, I, L, M, or V;
X_{10c} is Q, R, or T;
X_{11c} is A, G, or S;
X_{13c} is D, E, G, N, Q, or S;
X_{17c} is A, H, K, N, R, S, T, or V;
X_{21c} is L, M, R, or Y;
X_{22c} is A, I, N, Q, S, T, or V;
X_{23c} is A, E, F, I, K, L, N, R, T, or V;
X_{25c} is A, F, H, L, N, Q, S, T, or Y;
X_{26c} is A, I, L, M, N, R, S, T, V, or Y; and
X_{2c}, X_{3c}, X_{5c}, X_{7c}, X_{8c}, X_{9c}, X_{12c}, X_{16c}, X_{19c}, X_{20c}, and X_{24c} are each individually selected from any amino acid;
4) X_{1d}X_{2d}X_{3d}X_{4d}X_{5d}X_{6d}X_{7d}X_{8d}X_{9d}X_{10d}X_{11d}X_{12d}X_{13d}X_{14d}X_{15d}X_{16d}X_{17d}X_{18d}X_{19d}X_{20d}X_{21d}X_{22d}X_{23d}X_{24d}X_{25d}X_{26d}X_{27d}X_{28d}, wherein:
X_{1d} is E, K, N, T, G, S, L, D, V, A, R, or P;
X_{2d} is E, H, I, T, G, S, L, D, V, A, or P;
X_{4d} is M, I, T, S, L, V, A, R or P;
X_{5d} is E, K, N, I, T, G, S, D, Q, V, A, R, or P;
X_{6d} is E, G, S, D, A, R, or P;
X_{7d} is I, L, D, A, or R;
X_{8d} is M, H, K, T, L, V, Q, D, A, or R;
X_{9d} is E, K, I, T, G, S, L, D, Q, V, or A;
X_{10d} is E, K, H, D, Q, V, A, or R;
X_{11d} is M, H, I, S, L, V, Q, A, or R;
X_{12d} is Q, E, K, N, M, S, L, D, V, A, or R;
X_{13d} is E, K, H, G, S, L, D, Q, A, or R;
X_{14d} is E, Y, K, N, I, H, L, V, or A;
X_{16d} is E, K, I, T, G, S, L, D, Q, A, or R;
X_{17d} is E, K, H, T, G, D, Q, A, or R:
X_{19d} is Q, E, K, N, T, G, S, D, V, A, or R;
X_{20d} is Q, E, K, N, T, G, S, V, D, A, or R;
X_{21d} is I, S, W, L, V, F, A, or R;
X_{22d} is Q, E, M, T, G, S, L, V, D, or A;
X_{23d} is E, K, N, I, T, G, S, D, A, R, or P;
X_{24d} is E, M, I, L, D, Q, or A:
X_{25d} is E, Y, I, L, V, F, A, or R;
X_{26d} is E, M, T, G, S, L, D, V, A, or R;
X_{27d} is E, K, N, G, S, L, D, Q, A, or R;
X_{28d} is Q, E, G, V, D, A, R, or P; and
X_{3d}, X_{15d}, and X_{18d} are each individually selected from any amino acid;
5) X₁ₑX₂ₑX₃ₑX₄ₑX₅ₑX₆ₑX₇ₑX₈ₑX₉ₑX₁₀ₑX₁₁ₑX₁₂ₑX₁₃ₑX₁₄ₑX₁₅ₑX₁₆ₑX₁₇ₑX₁₈ₑ, wherein:
X₁ₑ is A, D, E, H, K, N, Q, R, or S;
X₂ₑ is A, D, E, F, G, H, K, M, N, Q, R, S, W, or Y;
X₃ₑ is E, F, or Y;
X₄ₑ is F, H, L, W, or Y;
X₆ₑ is A, D, E, F, I, K, L, M, N, Q, R, S, T, or Y;
X₇ₑ is F, I, Q, S, T, or V;
X₈ₑ is A, G, K, L, N, R, S, T, or V;
X₉ₑ is A, D, E, H, K, N, Q, R, T, or Y;
X₁₀ₑ is I, N, Q, or R;
X₁₁ₑ is F, I, L, M, Q, or S;
X₁₄ₑ is A, G, K, N, or S;
X₁₅ₑ is K, M, Q, R, S, T, or V;
X₁₈ₑ is A, E, G, K, M, N, S, T, or Y; and
X₅ₑ, X₁₂ₑ, X₁₃ₑ, X₁₆ₑ, and X₁₇ₑ are each individually selected from any amino acid;
6) WX_{2f}X_{3f}X_{4f}X_{5f}X_{6f}X_{7f}X_{8f}X_{9f}X_{10f}X_{11f}X_{12f}X_{13f}X_{14f}X_{15f}X_{16f}GX_{18f}X_{19f}X_{20f}X_{21f}X_{22f}X_{23f}, wherein:
X_{2f} is A, E, H, N, R, S, T, or V;
X_{4f} is A, G, N, S, or T;
X_{5f} is F, G, L, M, N, Q, S, T, or V;
X_{6f} is I, L, P, or V;
X_{9f} is I, L, T, or V;
X_{14f} is A, C, G, M, Q, R, S, or T;
X_{16f} is I, L, V, or Y;
X_{18f} is D, E, H, N, Q, or S;
X_{20f} is E, H, I, L, M, Q, R, or T;
X_{21f} is A, E, F, H, L, N, P, or Y;
X_{22f} is C, F, H, K, M, N, Q, R, T, or Y;
X_{23f} is D, E, F, I, K, L, N, Q, R, S, T, or V; and
X_{3f}, X_{7f}, X_{8f} X_{10f}, X_{11f}, X_{12f}, X_{13f}, X_{15f}, and X_{19f} are each individually selected from any amino acid;
7) X_{1g}X_{2g}X_{3g}X_{4g}X_{5g}EX_{7g}X_{8g}X_{9g}X_{10g}X_{11g}X_{12g}RX_{14g}X_{15g}X_{16g}X_{17g}X_{18g}X_{19g}X_{20g}X_{21g}, wherein:
X_{1g} is A, G, I, N, S, T, or V;
X_{3g} is A, I, or S;
X_{5g} is F, I, L, M, or Y;
X_{7g} is I or R;
X_{10g} is D, I, L, or T;
X_{12g} is A, E, I, K, M, Q, or S;
X_{14g} is I, T, or V;
X_{16g} is A, D, G, R, S, or T;
X_{18g} is F, K, L, M, or Y;
X_{19g} is A, E, H, I, K, L, M, N, Q, R, V, W, or Y;
X_{21g} is A, I, K, L, M, or R; and
X_{2g}, X_{4g}, X_{8g}, X_{9g}, X_{11g}, X_{15g}, X_{17g}, and X_{20g} are each individually selected from any amino acid;
8) X₁ₕX₂ₕX₃ₕX₄ₕX₅ₕX₆ₕX₇ₕX₈ₕX₉ₕX₁₀ₕX₁₁ₕ, wherein:
X₁ₕ is F or Y;
X₂ₕ is D, E, K, Q, or S;
X₃ₕ is E, K, L, M, or Q;
X₄ₕ is K, L, or R:
X₅ₕ is K, L, or V;
X₇ₕ is G or N;
X₈ₕ is D, E, H, K, L, M, or R;
X₉ₕ is S or T;
X₁₁ₕ is F, H, I, Q, S, T, V, or W; and
X₆ₕ and X₁₀ₕ are each individually selected from any amino acid;
9) X₁ᵢX₂ᵢX₃ᵢX₄ᵢX₅ᵢX₆ᵢX₇ᵢX₈ᵢX₉ᵢX₁₀ᵢX₁₁ᵢX₁₃ᵢX₁₄ᵢX₁₅ᵢX₁₆ᵢX₁₇ᵢX₁₈ᵢX₁₉ᵢX₂₀ᵢX₂₁ᵢX₂₂ᵢX₂₃ᵢX₂₄ᵢX₂₅ᵢX₂₆ᵢX₂₇ᵢ, wherein:
X₁ᵢ is I, L, or V;
X₄ᵢ is A, D, F, H, I, L, M, N, Q, S, V, or Y;
X₈ᵢ is A, G, or S;
X₁₀ᵢ is D, E, I, K, N, Q, R, or S;
X₁₁ᵢ is E or Q;
X₁₅ᵢ is A or K;
X₁₆ᵢ is A, Q, R, or S;
X₁₈ᵢ is L, M, or R;
X₁₉ᵢ is I, L, Q, R, S, or V;
X₂₁ᵢ is A, D, E, G, H, I, Q, R, or S;
X₂₂ᵢ is A, K, N, Q, S, T, or V;
X₂₃ᵢ is A, H, K, R, W, or Y;
X₂₅ᵢ is A, G, H, I, K, Q, R, S, or T;
X₂₇ᵢ is C, H, I, K, L, R, or V; and
X₂ᵢ, X₃ᵢ, X₅ᵢ, X₆ᵢ, X₇ᵢ, X₉ᵢ, X₁₃ᵢ, X₁₄ᵢ, X₁₇ᵢ, X₂₀ᵢ, X₂₄ᵢ, and X₂₆ᵢ are each individually selected from any amino acid;
10) RX₂ⱼX₃ⱼX₄ⱼW, wherein:
X₂ⱼ is L, M, Q, or R;
X₃ⱼ is A, N, or S; and
X₄ⱼ is N, P, S, or T;
11) X₁ₖX₂ₖX₃ₖX₄ₖX₅ₖX₆ₖX₇ₖX₈ₖF, wherein:
X₁ₖ is I, L, or V;
X₂ₖ is A or V;
X₄ₖ is A, F, H, I, L, Q, W, or Y;
X₅ₖ is I, M, or V;
X₇ₖ is E, L, Q, or T:
X₈ₖ is A, I, or V; and
X₃ₖ and X₆ₖ are each individually selected from any amino acid;
12) RX₂ₗX₃ₗX₄ₗX₅ₗX₆ₗX₇ₗX₈ₗX₉ₗX₁₀ₗX₁₁ₗX₁₂ₗX₁₃ₗ, wherein:
X₂ₗ is D, K, N, R, S, or V;
X₃ₗ is A, D, E, F, G, K, P, Q, or S;
X₄ₗ is A, E, I, K, L, S, T, or V;
X₅ₗ is any amino acid:
X₆ₗ is F, G, I, L, N, or V;
X₇ₗ is A, F, I, L, Q, R, V, or Y;
X₈ₗ is D, E, I, L, M, N, Q, S, T, or V;
X₉ₗ is D, E, F, I, L, M, Q, T, V, or Y;
X₁₀ₗ is I, K, L, R, or V;
X₁₁ₗ is D, E, K, N, Q, or R;
X₁₂ₗ is D, E, F, K, L, N, Q, W, or Y; and
X₁₃ₗ is F or L; and
13) X₁ₘX₂ₘX₃ₘX₄ₘX₅ₘX₆ₘX₇ₘX₈ₘX₉ₘX₁₀ₘX₁₁ₘX₁₂ₘX₁₃ₘX₁₄ₘX₁₅ₘX₁₆ₘX₁₇ₘX₁₈ₘX₁₉ₘX₂₀ₘX₂₁ₘX₂₂ₘX₂₃ₘX₂₄ₘ, wherein:
X₁ₘ is A, E, F, I, L, M, N, Q, S, T, V, or Y;
X₂ₘ is A, F, G, I, L, M, R, S, T, or V;
X₆ₘ is A, D, E, F, G, H, L, M, N, S, or T;
X₉ₘ is D, M, N, or S;
X₁₀ₘ is D, E, or Q;
X₁₂ₘ is C, F, H, L, T, V, or Y;
X₁₄ₘ is A, E, K, L, R, or Y;
X₁₇ₘ is A, L, or S;
X₁₉ₘ is D, E, K, N, Q, R, or S;
X₂₀ₘ is G, I, M, Q, R, T, or V;
X₂₁ₘ is D, H, K, N, Q, or R;
X₂₃ₘ is A, G, I, L, N, S, T, or V;
X₂₄ₘ is F, H, I, K, L, M, N, Q, V, W, or Y; and
X₃ₘ, X₄ₘ, X₅ₘ, X₇ₘ, X₈ₘ, X₁₁ₘ, X₁₃ₘ, X₁₅ₘ, X₁₆ₘ, X₁₈ₘ, and X₂₂ₘ, are each individually selected from any amino acid,
or a nucleic acid encoding thereof.

19. A method of altering a target DNA comprising contacting the target DNA with
a polypeptide comprising a recombinase having an amino acid sequence having at least 70% identity to any of SEQ ID NOs: 88-1183, or a nucleic acid encoding thereof.

20. The method of any of claims 17-19, wherein the target DNA comprises a donor recognition sequence, a recipient recognition sequence, or both.

21. The method of any of claims 17-20, further comprising contacting the target DNA with a first polynucleotide comprising a donor recognition sequence for the recombinase.

22. The method of claim 21, wherein the first polynucleotide further comprises a cargo DNA sequence.

23. The method of claim 22, wherein the cargo DNA sequence is greater than 1 kilobase pair.

24. The method of claim 22 or claim 23, wherein the cargo DNA sequence is greater than 5 kilobase pairs.

25. The method of any of claims 21-24, wherein the target DNA comprises a recipient attachment sequence configured to bind to the recombinase.

26. The method of any of claims 20-25, wherein the donor recognition sequence, the recipient recognition sequence or both are pseudo-recognition sequences.

27. The method of any of claims 17-26, wherein the target DNA sequence encodes a gene product.

28. The method of any of claims 17-27, wherein the target DNA is in a cell.

29. The method of claim 28, wherein the cell is a eukaryotic cell.

30. The method of claim 29, wherein the eukaryotic cell is a human cell.

31. The method of claim 28, wherein the cell is a prokaryotic cell.

32. The method of any of claims, 28-31, wherein the target DNA sequence is a genomic DNA sequence.

33. The method of any of claims 28-31, wherein the contacting comprises introducing into the cell.

34. The method of claim 33, wherein introducing into the cell comprises administering to a subject.

35. The method of claim 34, wherein the subject is a human.

36. The method of claim 34 or 35, wherein the administering comprises in vivo administration.

37. The method of claim 34 or 35, wherein the administering comprises transplantation of ex vivo treated cells comprising the system.

38. The method of any of claims 33-37, wherein the recombinase, or the nucleic acid encoding thereof, is introduced into the cell before, concurrently with, or after the introduction of the donor polynucleotide.

39. Use of the system of any of claims 1-12 or a composition of claim 13 to alter a target nucleic acid sequence.

40. The use of claim 39, wherein the target DNA comprises a donor recognition sequence, a recipient recognition sequence, or both.

41. The use of claim 39 or 40, further comprising contacting the target DNA with a first polynucleotide comprising a donor recognition sequence for the recombinase.

42. The use of claim 41, wherein the first polynucleotide further comprises a cargo DNA sequence.

43. The use of claim 42, wherein the cargo DNA sequence is greater than 1 kilobase pair.

44. The use of claim 42 or claim 43, wherein the cargo DNA sequence is greater than 5 kilobase pairs.

45. The use of any of claims 41-44, wherein the target DNA comprises a recipient attachment sequence configured to bind to the recombinase.

46. The use of any of claims 40-45, wherein the donor recognition sequence, the recipient recognition sequence or both are pseudo-recognition sequences.

47. The use of any of claims 39-46, wherein the target DNA sequence encodes a gene product.

48. The use of any of claims 39-47, wherein the target DNA is in a cell.

49. The use of claim 48, wherein the cell is a eukaryotic cell.

50. The use of claim 49, wherein the eukaryotic cell is a human cell.

51. The use of claim 48, wherein the cell is a prokaryotic cell.

52. The use of any of claims, 48-51, wherein the target DNA sequence is a genomic DNA sequence.

53. The use of any of claims 48-51, wherein the contacting comprises introducing into the cell.

54. The use of claim 53, wherein introducing into the cell comprises administering to a subject.

55. The use of claim 54, wherein the subject is a human.

56. The use of claim 54 or 55, wherein the administering comprises in vivo administration.

57. The use of claim 54 or 55, wherein the administering comprises transplantation of ex vivo treated cells comprising the system.

58. The use of any of claims 53-57, wherein the recombinase, or the nucleic acid encoding thereof, is introduced into the cell before, concurrently with, or after the introduction of the donor polynucleotide.
